(19) United States
(12) Patent Application Publication
(10) Pub. No.: US 2003/0072767 A1
(43) Pub. Date: Apr. 17, 2003

(54) Compositions and methods for WT1 specific immunotherapy

(76) Inventors: Alexander Gaiger, Seattle, WA (US); Patricia D. McNeill, Federal Way, WA (US); Molly Smithgall, Seattle, WA (US); Gus Moulton, Seattle, WA (US); Thomas S. Vedvick, Federal Way, WA (US); Paul R. Sleath, Seattle, WA (US); Sally Mossman, Seattle, WA (US); Lawrence Evans, Seattle, WA (US); A. Gregory Spies, Shoreline, WA (US); Jeremy Boydston, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/938,864

(22) Filed: Aug. 24, 2001

Related U.S. Application Data
(63) Continuation-in-part of application No. 09/785,019, filed on Feb. 15, 2001.
Application No. 09/785,019 is a continuation-in-part of application No. 09/685,830, filed on Oct. 9, 2000.
Application No. 09/685,830 is a continuation-in-part of application No. 09/684,361, filed on Oct. 6, 2000.
Application No. 09/684,361 is a continuation-in-part of application No. 09/276,484, filed on Mar. 25, 1999.
Application No. 09/276,484 is a continuation-in-part of application No. 09/164,223, filed on Sep. 30, 1998.

(51) Int. Cl. A61K 39/00; C07H 21/04; C12N 9/64; C12P 21/02; C12N 5/06

(52) U.S. Cl. 424/185.1; 435/69.1; 435/226; 435/320.1; 435/325; 536/23.2

(57) ABSTRACT

Compositions and methods for the therapy of malignant diseases, such as leukemia and cancer, are disclosed. The compositions comprise one or more of a WT1 polynucleotide, a WT1 polypeptide, an antigen-presenting cell presenting a WT1 polypeptide, an antibody that specifically binds to a WT1 polypeptide; or a T cell that specifically reacts with a WT1 polypeptide. Such compositions may be used, for example, for the prevention and treatment of metastatic diseases.

### CROSS-REFERENCE TO RELATED APPLICATIONS

This application is continuation-in-part of U.S. patent application Ser. No. 09/785,019, filed Feb. 15, 2001 which is a continuation-in-part of U.S. patent application Ser. No. 09/685, 830, filed Oct. 9, 2000; which is a continuation-in-part of U. S. application Ser. No. 09/684,361, filed Oct. 6, 2000; which is a continuation-in-part of U.S. application Ser. No. 09/276,484, filed Mar. 25, 1999; which is a continuation-in-part of U.S. application Ser. No. 09/164,223, filed Sep. 30, 1998, and are incorporated herein by reference in their entirety.

### STATEMENT REGARDING FEDERALLY SPONSORED RESEARCH OR DEVELOPMENT

This invention was made in part with government support under NIH SBIR Phase I grant number IR43 CA81752-01A1. The Government may have certain rights in this invention.

### BACKGROUND OF THE INVENTION

1. Field of the Invention

The present invention relates generally to the immunotherapy of malignant diseases such as leukemia and cancers. The invention is more specifically related to compositions for generating or enhancing an immune response to WT1, and to the use of such compositions for preventing and/or treating malignant diseases.

2. Description of the Related Art

Cancer and leukemia are significant health problems in the United States and throughout the world. Although advances have been made in detection and treatment of such diseases, no vaccine or other universally successful method for prevention or treatment of cancer and leukemia is currently available. Management of the diseases currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and the high mortality continues to be observed in many cancer patients.

Immunotherapies have the potential to substantially improve cancer and leukemia treatment and survival. Recent data demonstrate that leukemia can be cured by immunotherapy in the context of bone marrow transplantation (e.g., donor lymphocyte infusions). Such therapies may involve the generation or enhancement of an immune response to a tumor-associated antigen (TAA). However, to date relatively few TAAs are known and the generation of an immune response against such antigens has, with rare exception, not been shown to be therapeutically beneficial.

Accordingly, there is a need in the art for improved methods for leukemia and cancer prevention and therapy. The present invention fulfills these needs and further provides other related advantages.

### BRIEF SUMMARY OF THE INVENTION

Briefly stated, this invention provides compositions and methods for the diagnosis and therapy of diseases such as leukemia and cancer. In one aspect, the present invention provides polypeptides comprising an immunogenic portion of a native WT1, or a variant thereof that differs in one or more substitutions, deletions, additions and/or insertions such that the ability of the variant to react with antigen-specific antisera and/or T-cell lines or clones is not substantially diminished. Within certain embodiments, the polypeptide comprises no more than 16 consecutive amino acid residues of a native WT1 polypeptide. Within other embodiments, the polypeptide comprises an immunogenic portion of amino acid residues 1-174 of a native WT1 polypeptide or a variant thereof, wherein the polypeptide comprises no more than 16 consecutive amino acid residues present within amino acids 175 to 449 of the native WT1 polypeptide. The immunogenic portion preferably binds to an MHC class I and/or class II molecule. Within certain embodiments, the polypeptide comprises a sequence selected from the group consisting of (a) sequences recited in any one or more of Tables II-XLVI, (b) variants of the foregoing sequences that differ in one or more substitutions, deletions, additions and/or insertions such that the ability of the variant to react with antigen-specific antisera and/or T-cell lines or clones is not substantially diminished and (c) mimetics of the polypeptides recited above, such that the ability of the mimetic to react with antigen-specific antisera and/or T cell lines or clones is not substantially diminished.

Within other embodiments, the polypeptide comprises a sequence selected from the group consisting of (a) ALLPAVPSL (SEQ ID NO:34), GATLKGVAA (SEQ ID NO:88), CMTWNQMNL (SEQ ID NOs: 49 and 258), SCLESQPTI (SEQ ID NOs: 199 and 296), SCLESQPAI (SEQ ID NO:198), NLYQMTSQL (SEQ ID NOs: 147 and 284), ALLPAVSSL (SEQ ID NOs: 35 and 255), RMFPNAPYL (SEQ ID NOs: 185 and 293), VLDFAPPGA (SEQ ID NO:241), VLDFAPPGAS (SEQ ID NO:411), (b) variants of the foregoing sequences that differ in one or more substitutions, deletions, additions and/or insertions such that the ability of the variant to react with antigen-specific antisera and/or T-cell lines or clones is not substantially diminished and (c) mimetics of the polypeptides recited above, such that the ability of the mimetic to react with antigen-specific antisera and/or T cell lines or clones is not substantially diminished. Mimetics may comprises amino acids in combination with one or more amino acid mimetics or may be entirely nonpeptide mimetics.

Within further aspects, the present invention provides polypeptides comprising a variant of an immunogenic portion of a WT1 protein, wherein the variant differs from the immunogenic portion due to substitutions at between 1 and 3 amino acid positions within the immunogenic portion such that the ability of the variant to react with anligen-specific antisera and/or T-cell lines or clones is enhanced relative to a native WT1 protein.

The present invention further provides WT1 polynucleotides that encode a WT1 polypeptide as described above.

Within other aspects, the present invention provides pharmaceutical compositions and vaccines. Pharmaceutical compositions may comprise a polypeptide or mimetic as described above and/or one or more of (i) a WT1 polynucleotide; (ii) an antibody or antigen-binding fragment thereof that specifically binds to a WT1 polypeptide; (iii) a T cell that specifically reacts with a WT1 polypeptide or (iv) an antigen-presenting cell that expresses a WT1 polypeptide, in combination with a pharmaceutically acceptable carrier or excipient. Vaccines comprise a polypeptide as described above and/or one or more of (i) a WT1 polynucleotide, (ii) an antigen-presenting cell that expresses a WT1 polypeptide or (iii) an anti-idiotypic antibody, and a non-specific immune response enhancer. Within certain embodiments, less than 23 consecutive amino acid residues, preferably less than 17 amino acid residues, of a native WT1 polypeptide are present within a WT1 polypeptide employed within such pharmaceutical compositions and vaccines. The immune response enhancer may be an adjuvant. Preferably, an immune response enhancer enhances a T cell response.

The present invention further provides methods for enhancing or inducing an immune response in a patient, comprising administering to a patient a pharmaceutical composition or vaccine as described above. In certain embodiments, the patient is a human.

The present invention further provides methods for inhibiting the development of a malignant disease in a patient, comprising administering to a patient a pharmaceutical composition or vaccine as described above. Malignant diseases include, but are not limited to leukemias (e.g., acute myeloid, acute lymphocytic and chronic myeloid) and cancers (e.g., breast, lung, thyroid or gastrointestinal cancer or a melanoma). The patient may, but need not, be afflicted with the malignant disease, and the administration of the pharmaceutical composition or vaccine may inhibit the onset of such a disease, or may inhibit progression and/or metastasis of an existing disease.

The present invention further provides, within other aspects, methods for removing cells expressing WT1 from bone marrow and/or peripheral blood or fractions thereof, comprising contacting bone marrow, peripheral blood or a fraction of bone marrow or peripheral blood with T cells that specifically react with a WT1 polypeptide, wherein the step of contacting is performed under conditions and for a time sufficient to permit the removal of WT1 positive cells to less than 10%, preferably less than 5% and more preferably less than 1%, of the number of myeloid or lymphatic cells in the bone marrow, peripheral blood or fraction. Bone marrow, peripheral blood and fractions may be obtained from a patient afflicted with a disease associated with WT1 expression, or may be obtained from a human or non-human mammal not afflicted with such a disease.

Within related aspects, the present invention provides methods for inhibiting the development of a malignant disease in a patient, comprising administering to a patient bone marrow, peripheral blood or a fraction of bone marrow or peripheral blood prepared as described above. Such bone marrow, peripheral blood or fractions may be autologous, or may be derived from a related or unrelated human or non-human animal (e.g., syngeneic or allogeneic).

In other aspects, the present invention provides methods for stimulating (or priming) and/or expanding T cells, comprising contacting T cells with a WT1 polypeptide under conditions and for a time sufficient to permit the stimulation and/or expansion of T cells. Such T cells may be autologous, allogeneic, syngeneic or unrelated WT1-specific T cells, and may be stimulated in vitro or in vivo. Expanded T cells may, within certain embodiments, be present within bone marrow, peripheral blood or a fraction of bone marrow or peripheral blood, and may (but need not) be clonal. Within certain embodiments, T cells may be present in a mammal during stimulation and/or expansion. WT1 -specific T cells may be used, for example, within donor lymphocyte infusions.

Within related aspects, methods are provided for inhibiting the development of a malignant disease in a patient, comprising administering to a patient T cells prepared as described above. Such T cells may, within certain embodiments, be autologous, syngeneic or allogeneic.

The present invention further provides, within other aspects, methods for monitoring the effectiveness of an immunization or therapy for a malignant disease associated with WT1 expression in a patient. Such methods are based on monitoring antibody, CD4+ T cell and/or CD8+ T cell responses in the patient. Within certain such aspects, a method may comprise the steps of: (a) incubating a first biological sample with one or more of: (i) a WT1 polypeptide; (ii) a polynucleotide encoding a WT1 polypeptide; or (iii) an antigen presenting cell that expresses a WT1 polypeptide, wherein the first biological sample is obtained from a patient prior to a therapy or immunization, and wherein the incubation is performed under conditions and for a time sufficient to allow immunocomplexes to form; (b) detecting immunocomplexes formed between the WT1 polypeptide and antibodies in the biological sample that specifically bind to the WT1 polypeptide; (c) repeating steps (a) and (b) using a second biological sample obtained from the same patient following therapy or immunization; and (d) comparing the number of immunocomplexes detected in the first and second biological samples, and therefrom monitoring the effectiveness of the therapy or immunization in the patient.

Within certain embodiments of the above methods, the step of detecting comprises (a) incubating the immunocomplexes with a detection reagent that is capable of binding to the immunocomplexes, wherein the detection reagent comprises a reporter group, (b) removing unbound detection reagent, and (c) detecting the presence or absence of the reporter group. The detection reagent may comprise, for example, a second antibody, or antigen-binding fragment thereof, capable of binding to the antibodies that specifically bind to the WT1 polypeptide or a molecule such as Protein A. Within other embodiments, a reporter group is bound to the WT1 polypeptide, and the step of detecting comprises removing unbound WT1 polypeptide and subsequently detecting the presence or absence of the reporter group.

Within further aspects, methods for monitoring the effectiveness of an immunization or therapy for a malignant disease associated with WT1 expression in a patient may comprise the steps of: (a) incubating a first biological sample with one or more of: (i) a WT1 polypeptide; (ii) a polynucleotide encoding a WT1 polypeptide; or (iii) an antigen presenting cell that expresses a WT1 polypeptide, wherein the biological sample comprises CD4+ and/or CD8+ T cells and is obtained from a patient prior to a therapy or immunization, and wherein the incubation is performed under conditions and for a time sufficient to allow specific activation, proliferation and/or lysis of T cells; (b) detecting an amount of activation, proliferation and/or lysis of the T cells; (c) repeating steps (a) and (b) using a second biological sample comprising CD4+ and/or CD8+ T cells, wherein the second biological sample is obtained from the same patient following therapy or immunization; and (d) comparing the amount of activation, proliferation and/or lysis of T cells in the first and second biological samples, and therefrom monitoring the effectiveness of the therapy or immunization in the patient.

The present invention further provides methods for inhibiting the development of a malignant disease associated with WT1 expression in a patient, comprising the steps of: (a) incubating CD4⁺ and/or CD8+ T cells isolated from a patient with one or more of: (i) a WT1 polypeptide; (ii) a polynucleotide encoding a WT1 polypeptide; or (iii) an antigen presenting cell that expresses a WT1 polypeptide, such that the T cells proliferate; and (b) administering to the patient an effective amount of the proliferated T cells, and therefrom inhibiting the development of a malignant disease in the patient. Within certain embodiments, the step of incubating the T cells may be repeated one or more times.

Within other aspects, the present invention provides methods for inhibiting the development of a malignant disease associated with WT1 expression in a patient, comprising the steps of: (a) incubating CD4⁺ and/or CD8+ T cells isolated from a patient with one or more of: (i) a WT1 polypeptide; (ii) a polynucleotide encoding a WT1 polypeptide; or (iii) an antigen presenting cell that expresses a WT1 polypeptide, such that the T cells proliferate; (b) cloning one or more cells that proliferated; and (c) administering to the patient an effective amount of the cloned T cells.

Within other aspects, methods are provided for determining the presence or absence of a malignant disease associated with WT1 expression in a patient, comprising the steps of: (a) incubating CD4⁺ and/or CD8+ T cells isolated from a patient with one or more of: (i) a WT1 polypeptide; (ii) a polynucleotide encoding a WT1 polypeptide; or (iii) an antigen presenting cell that expresses a WT1 polypeptide; and (b) detecting the presence or absence of specific activation of the T cells, therefrom determining the presence or absence of a malignant disease associated with WT1 expression. Within certain embodiments, the step of detecting comprises detecting the presence or absence of proliferation of the T cells.

Within further aspects, the present invention provides methods for determining the presence or absence of a malignant disease associated with WT1 expression in a patient, comprising the steps of: (a) incubating a biological sample obtained from a patient with one or more of: (i) a WT1 polypeptide; (ii) a polynucleotide encoding a WT1 polypeptide; or (iii) an antigen presenting cell that expresses a WT1 polypeptide, wherein the incubation is performed under conditions and for a time sufficient to allow immunocomplexes to form; and (b) detecting immunocomplexes formed between the WT1 polypeptide and antibodies in the biological sample that specifically bind to the WT1 polypeptide; and therefrom determining the presence or absence of a malignant disease associated with WT1 expression.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 depicts a comparison of the mouse (MO) and human (HU) WT1 protein sequences (SEQ ID NOS: 320 and 319 respectively).

FIG. 2 is a Western blot illustrating the detection of WT1 specific antibodies in patients with hematological malignancy (AML). Lane 1 shows molecular weight markers; lane 2 shows a positive control (WT1 positive human leukemia cell line immunoprecipitated with a WT1 specific antibody); lane 3 shows a negative control (WT1 positive cell line immunoprecipitated with mouse sera); and lane 4 shows a WT1 positive cell line immunoprecipitated with sera of a patient with AML. For lanes 2-4, the immunoprecipitate was separated by gel electrophoresis and probed with a WT1 specific antibody.

FIG. 3 is a Western blot illustrating the detection of a WT1 specific antibody response in B6 mice immunized with TRAMP-C, a WT1 positive tumor cell line. Lanes 1, 3 and 5 show molecular weight markers, and lanes 2, 4 and 6 show a WT1 specific positive control (N180, Santa Cruz Biotechnology, polypeptide spanning 180 amino acids of the N-terminal region of the WT1 protein, migrating on the Western blot at 52 kD). The primary antibody used was WT180 in lane 2, sera of non-immunized B6 mice in lane 4 and sera of the immunized B6 mice in lane 6.

FIG. 4 is a Western blot illustrating the detection of WT1 specific antibodies in mice immunized with representative WT1 peptides. Lanes 1, 3 and 5 show molecular weight markers and lanes 2, 4 and 6 show a WT1 specific positive control (N180, Santa Cruz Biotechnology, polypeptide spanning 180 amino acids of the N-terminal region of the WT1 protein, migrating on the Western blot at 52 kD). The primary antibody used was WT180 in lane 2, sera of non-immunized B6 mice in lane 4 and sera of the immunized B6 mice in lane 6.

FIGS. 5A to 5C are graphs illustrating the stimulation of proliferative T cell responses in mice immunized with representative WT1 peptides. Thymidine incorporation assays were performed using one T cell line and two different clones, as indicated, and results were expressed as cpm. Controls indicated on the x axis were no antigen (No Ag) and B6/media; antigens used were p6-22 human (pl), p117-139 (p2) or p244-262 human (p3).

FIGS. 6A and 6B are histograms illustrating the stimulation of proliferative T cell responses in mice immunized with representative WT1 peptides. Three weeks after the third immunization, spleen cells of mice that had been inoculated with Vaccine A or Vaccine B were cultured with medium alone (medium) or spleen cells and medium (B6/no antigen), B6 spleen cells pulsed with the peptides p6-22 (p6), p117-139 (p117), p244-262 (p244) (Vaccine A; FIG. 6A) or p287-301 (p287), p299-313 (p299), p421-435 (p421) (Vaccine B; FIG. 6B) and spleen cells pulsed with an irrelevant control peptide (irrelevant peptide) at 25 ug/ml and were assayed after 96 hr for proliferation by (³H) thymidine incorporation. Bars represent the stimulation index (SI), which is calculated as the mean of the experimental wells divided by the mean of the control (B6 spleen cells with no antigen).

FIGS. 7A-7D are histograms illustrating the generation of proliferative T-cell lines and clones specific for p117-139 and p6-22. Following in vivo immunization, the initial three in vitro stimulations (IVS) were carried out using all three peptides of Vaccine A or B, respectively. Subsequent IVS were carried out as single peptide stimulations using only the two relevant peptides p117-139 and p6-22. Clones were derived from both the p6-22 and p117-139 specific T cell lines, as indicated. T cells were cultured with medium alone (medium) or spleen cells and medium (B6/no antigen), B6 spleen cells pulsed with the peptides p6-22 (p6), p117-139 (p117) or an irrelevant control peptide (irrelevant peptide) at 25 ug/ml and were assayed after 96 hr for proliferation by (³H) thymidine incorporation. Bars represent the stimulation index (SI), which is calculated as the mean of the experimental wells divided by the mean of the control (B6 spleen cells with no antigen).

FIGS. 8A and 8B present the results of TSITES Analysis of human WT1 (SEQ ID NO:319) for peptides that have the potential to elicit Th responses. Regions indicated by “A” are AMPHI midpoints of blocks, “R” indicates residues matching the Rothbard/'Taylor motif, “D” indicates residues matching the IAd motif, and ‘d’ indicates residues matching the IEd motif.

FIGS. 9A and 9B are graphs illustrating the elicitation of WT1 peptide-specific CTL in mice immunized with WT1 peptides.

FIG. 9A illustrates the lysis of target cells by allogeneic cell lines and

FIG. 9B shows the lysis of peptide coated cell lines. In each case, the % lysis (as determined by standard chromium release assays) is shown at three indicated effector:target ratios. Results are provided for lymphoma cells (LSTRA and E10), as well as E10+p235-243 (E10+P235). E10 cells are also referred to herein as EL-4 cells.

FIGS. 10A-10D are graphs illustrating the elicitation of WT1 specific CTL, which kill WT1 positive tumor cell lines but do not kill WT1 negative cell lines, following vaccination of B6 mice with WT1 peptide P117.

FIG. 10A illustrates that T-cells of non-immunized B6 mice do not kill WT1 positive tumor cell lines.

FIG. 10B illustrates the lysis of the target cells by allogeneic cell lines.

FIGS. 10C and 10D demonstrate the lysis of WT1 positive tumor cell lines, as compared to WT1 negative cell lines in two different experiments. In addition, FIGS. 10C and 10D show the lysis of peptide-coated cell lines (WT1 negative cell line E10 coated with the relevant WT1 peptide P117) In each case, the % lysis (as determined by standard chromium release assays) is shown at three indicated effector:target ratios. Results are provided for lymphoma cells (E10), prostate cancer cells (TRAMP-C), a transformed fibroblast cell line (BLK-SV40), as well as E10+p117.

FIGS. 11A and 11B are histograms illustrating the ability of representative peptide P117-139 specific CTL to lyse WT1 positive tumor cells. Three weeks after the third immunization, spleen cells of mice that had been inoculated with the peptides p235-243 or p117-139 were stimulated in vitro with the relevant peptide and tested for ability to lyse targets incubated with WT1 peptides as well as WT1 positive and negative tumor cells. The bars represent the mean % specific lysis in chromium release assays performed in triplicate with an E:T ratio of 25:1.

FIG. 11A shows the cytotoxic activity of the p235-243 specific T cell line against the WT1 negative cell line EL-4 (EL-4, WT1 negative); EL-4 pulsed with the relevant (used for immunization as well as for restimulation) peptide p235-243 (EL-4+p235); EL-4 pulsed with the irrelevant peptides p117-139 (EL-4+p117), p126-134 (EL-4+p126) or p130-138 (EL-4+p130) and the WT1 positive tumor cells BLK-SV40 (BLK-SV40, WT1 positive) and TRAMP-C (TRAMP-C, WT1 positive), as indicated.

FIG. 11B shows cytotoxic activity of the p117-139 specific T cell line against EL-4; EL-4 pulsed with the relevant peptide P117-139 (EL-4+p117) and EL-4 pulsed with the irrelevant peptides p123-131 (EL-4+p123), or p128-136 (EL-4+p128); BLK-SV40 and TRAMP-C, as indicated.

FIGS. 12A and 12B are histograms illustrating the specificity of lysis of WT1 positive tumor cells, as demonstrated by cold target inhibition. The bars represent the mean % specific lysis in chromium release assays performed in triplicate with an E:T ratio of 25:1.

FIG. 12A shows the cytotoxic activity of the p117-139 specific T cell line against the WT1 negative cell line EL-4 (EL-4, WT1 negative); the WT1 positive tumor cell line TRAMP-C (TRAMP-C, WT1 positive); TRAMP-C cells incubated with a ten-fold excess (compared to the hot target) of EL-4 cells pulsed with the relevant peptide p117-139 (TRAMP-C+p117 cold target) without ⁵¹Cr labeling and TRAMP-C cells incubated with EL-4 pulsed with an irrelevant peptide without ⁵¹Cr labeling (TRAMP-C+irrelevant cold target), as indicated.

FIG. 12B shows the cytotoxic activity of the p117-139 specific T cell line against the WT1 negative cell line EL-4 (EL-4, WT1 negative); the WT1 positive tumor cell line BLK-SV40 (BLK-SV40, WT1 positive); BLK-SV40 cells incubated with the relevant cold target (BLK-SV40 +p117 cold target) and BLK-SV40 cells incubated with the irrelevant cold target (BLK-SV40 +irrelevant cold target), as indicated.

FIGS. 13A-13C are histograms depicting an evaluation of the 9mer CTL epitope within p117-139. The p117-139 tumor specific CTL line was tested against peptides within aa117-139 containing or lacking an appropriate H-2^{b} class I binding motif and following restimulation with p126-134 or p130-138. The bars represent the mean % specific lysis in chromium release assays performed in triplicate with an E:T ratio of 25:1.

FIG. 13A shows the cytotoxic activity of the p117-139 specific T cell line against the WT1 negative cell line EL-4 (EL-4, WT1 negative) and EL-4 cells pulsed with the peptides p117-139 (EL-4+p117), p119-127 (EL-4+p119), p120-128 (EL-4+p120), p123-131 (EL-4+p123), p126-134 (EL-4 +p126), p128-136 (EL-4+p128), and p130-138 (EL-4 +p130).

FIG. 13B shows the cytotoxic activity of the CTL line after restimulation with p126-134 against the WT1 negative cell line EL-4, EL-4 cells pulsed with p117-139 (EL-4+p117), p126-134 (EL-4+p126) and the WT1 positive tumor cell line TRAMP-C.

FIG. 13C shows the cytotoxic activity of the CTL line after restimulation with p130-138 against EL-4, EL-4 cells pulsed with p117-139 (EL-4+p117), p130-138 (EL-4+p130) and the WT1 positive tumor cell line TRAMP-C.

FIG. 14 depicts serum antibody reactivity to WT1 in 63 patients with AML. Reactivity of serum antibody to WT1/N-terminus protein was evaluated by ELISA in patients with AML. The first and second lanes represent the positive and negative controls, respectively. The first and second lanes represent the ositive and negative controls, respectively. Commercially obtained WT1 specific antibody WT180 was used for the positive control. The next 63 lanes represent results using sera from each individual patient. The OD values depicted were from ELISA using a 1:500 serum dilution. The figure includes cumulative data from 3 separate experiments.

FIG. 15 depicts serum antibody reactivity to WT1 proteins and control proteins in 2 patients with AML. Reactivity of serum antibody to WT1/full-length, WT1N-terminus, TRX and Ra12 proteins was evaluated by ELISA in 2 patients with AML. The OD values depicted were from ELISA using a 1:500 serum dilution. AML-1 and AML-2 denote serum from 2 of the individual patients in FIG. 1 with demonstrated antibody reactivity to WT1/full-length. The WT1 full-length protein was expressed as a fusion protein with Ra12. The WT1/N-terminus protein was expressed as a fusion protein with TRX. The control Ra12 and TRX proteins were purified in a similar manner. The results confirm that the serum antibody reactivity against the WT1 fusion proteins is directed against the WT1 portions of the protein.

FIG. 16 depicts serum antibody reactivity to WT1 in 81 patients with CML. Reactivity of serum antibody to WT1/full-length protein was evaluated by ELISA in patients with AML. The first and second lanes represent the positive and negative controls, respectively. Commercially obtained WT1 specific antibody WT180 was used for the positive control. The next 81 lanes represent results using sera from each individual patient. The OD values depicted were from ELISA using a 1:500 serum dilution. The figure includes cumulative data from 3 separate experiments.

FIG. 17 depicts serum antibody reactivity to WT1 proteins and control proteins in 2 patients with CML. Reactivity of serum antibody to WT1/full-length, WT1/N-terminus, TRX and Ra12 proteins was evaluated by ELISA in 2 patients with CML. The OD values depicted were from ELISA using a 1:500 serum dilution. CML-l and CML-2 denote serum from 2 of the individual patients in FIG. 3 with demonstrated antibody reactivity to WT1/full-length. The WT1/full-length protein was expressed as a fusion protein with Ra12. The WT1/N-terminus protein was expressed as a fusion protein with TRX. The control Ra12 and TRX proteins were purified in a similar manner. The results confirm that the serum antibody reactivity against the WT1 fusion proteins is directed against the WT1 portions of the protein.

FIG. 18 provides the characteristics of the recombinant WT1 proteins used for serological analysis.

FIG. 19A-19E is a bar graph depicting the antibody responses in mice elicited by vaccination with different doses of WT1 protein.

FIG. 20 is a bar graph of the proliferative T-cell responses in mice immunized with WT1 protein.

FIG. 21 is a photograph of human DC, examined by fluorescent microscopy, expressing WT1 following adeno WT1 and Vaccinia WT1 infection.

FIG. 22 is a photograph that demonstrates that WT1 expression in human DC is reproducible following adeno WT1 infection and is not induced by a control Adeno infection.

FIG. 23 is a graph of an IFN-gamma ELISPOT assay showing that WT1 whole gene in vitro priming elicits WT1 specific T-cell responses.

### DETAILED DESCRIPTION OF THE INVENTION

As noted above, the present invention is generally directed to compositions and methods for the immunotherapy and diagnosis of malignant diseases. The compositions described herein may include WT1 polypeptides, WT1 polynucleotides, antigen-presenting cells (APC, e.g., dendritic cells) that express a WT1 polypeptide, agents such as antibodies that bind to a WT1 polypeptide and/or immune system cells (e.g., T cells) specific for WT1. WT1 Polypeptides of the present invention generally comprise at least a portion of a Wilms Tumor gene product (WT1) or a variant thereof. Nucleic acid sequences of the subject invention generally comprise a DNA or RNA sequence that encodes all or a portion of such a polypeptide, or that is complementary to such a sequence. Antibodies are generally immune system proteins, or antigen-binding fragments thereof, that are capable of binding to a portion of a WT1 polypeptide. T cells that may be employed within such compositions are generally T cells (e.g., CD4⁺ and/or CD8⁺) that are specific for a WT1 polypeptide. Certain methods described herein further employ antigen-presenting cells that express a WT1 polypeptide as provided herein.

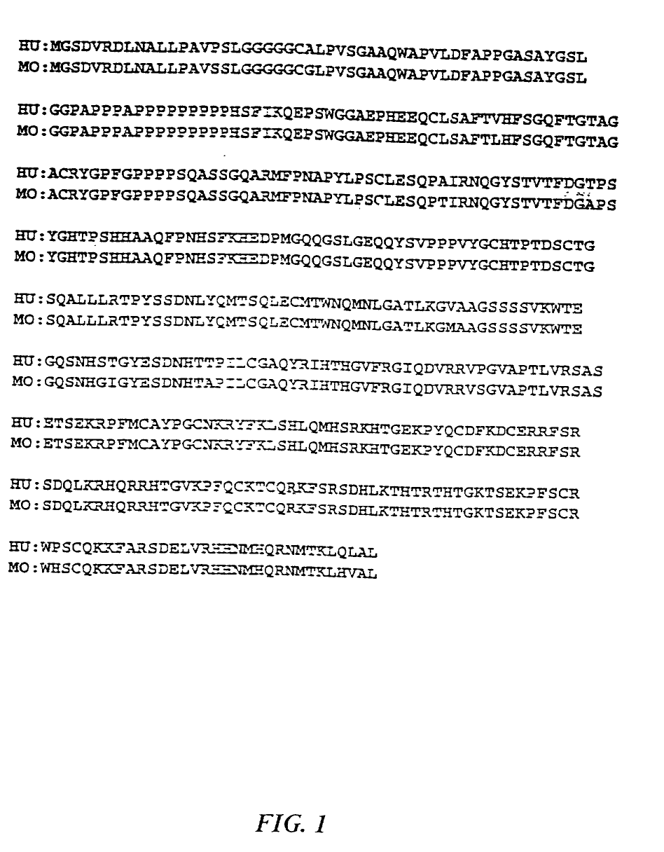

The present invention is based on the discovery that an immune response raised against a Wilms Tumor (WT) gene product (e.g., WT1) can provide prophylactic and/or therapeutic benefit for patients afflicted with malignant diseases characterized by increased WT1 gene expression. Such diseases include, but are not limited to, leukemias (e.g., acute myeloid leukemia (AML), chronic myeloid leukemia (CML), acute lymphocytic leukemia (ALL) and childhood ALL), as well as many cancers such as lung, breast, thyroid and gastrointestinal cancers and melanomas. The WT1 gene was originally identified and isolated on the basis of a cytogenetic deletion at chromosome 11p13 in patients with Wilms' tumor (see Call et al., U.S. Pat. No. 5,350,840). The gene consists of 10 exons and encodes a zinc finger transcription factor, and sequences of mouse and human WT1 proteins are provided in FIG. 1 and SEQ ID NOs: 319 and 320.

WT1 Polypeptides

Within the context of the present invention, a WT1 polypeptide is a polypeptide that comprises at least an immunogenic portion of a native WT1 (i.e., a WT1 protein expressed by an organism that is not genetically modified), or a variant thereof, as described herein. A WT1 polypeptide may be of any length, provided that it comprises at least an immunogenic portion of a native protein or a variant thereof. In other words, a WT1 polypeptide may be an oligopeptide (i.e., consisting of a relatively small number of amino acid residues, such as 8-10 residues, joined by peptide bonds), a full length WT1 protein (e.g., present within a human or non-human animal, such as a mouse) or a polypeptide of intermediate size. Within certain embodiments, the use of WT1 polypeptides that contain a small number of consecutive amino acid residues of a native WT1 polypeptide is preferred. Such polypeptides are preferred for certain uses in which the generation of a T cell response is desired. For example, such a WT1 polypeptide may contain less than 23, preferably no more than 18, and more preferably no more than 15 consecutive amino acid residues, of a native WT1 polypeptide. Polypeptides comprising nine consecutive amino acid residues of a native WT1 polypeptide are generally suitable for such purposes. Additional sequences derived from the native protein and/or heterologous sequences may be present within any WT1 polypeptide, and such sequences may (but need not) possess further immunogenic or antigenic properties. Polypeptides as provided herein may further be associated (covalently or noncovalently) with other polypeptide or non-polypeptide compounds.

An “immunogenic portion,” as used herein is a portion of a polypeptide that is recognized (i.e., specifically bound) by a B-cell and/or T-cell surface antigen receptor. Certain preferred immunogenic portions bind to an MHC class I or class II molecule. As used herein, an immunogenic portion is said to “bind to” an MHC class I or class II molecule if such binding is detectable using any assay known in the art. For example, the ability of a polypeptide to bind to MHC class I may be evaluated indirectly by monitoring the ability to promote incorporation of ¹²⁵I labeled β-microglobulin (β2m) into MHC class I/β2m/peptide heterotrimeric complexes (see Parker et al., J. Immunol. 152:163, 1994). Alternatively, functional peptide competition assays that are known in the art may be employed. Certain immunogenic portions have one or more of the sequences recited within one or more of Tables II-XIV. Representative immunogenic portions include, but are not limited to, RDLNALLPAVPSLGGGG (human WT1 residues 6-22; SEQ ID NO:1), PSQASSGQARMFPNAPYLPSCLE (human and mouse WT1 residues 117-139; SEQ ID NOs: 2 and 3 respectively), GATLKGVAAGSSSSVKWTE (human WT1 residues 244-262; SEQ ID NO:4), GATLKGVAA (human WT1 residues 244-252; SEQ ID NO:88), CMTWNQMNL (human and mouse WT1 residues 235-243; SEQ ID NOs: 49 and 258 respectively), SCLESQPTI (mouse WT1 residues 136-144; SEQ ID NO:296), SCLESQPAI (human WT1 residues 136-144; SEQ ID NO:198), NLYQMTSQL (human and mouse WT1 residues 225-233; SEQ ID NOs: 147 and 284 respectively); ALLPAVSSL (mouse WT1 residues 10-18, SEQ ID NO:255); RMFPNAPYL (human and mouse WT1 residues 126-134; SEQ ID NOs: 185 and 293 respectively), VLDFAPPGA (human WT1 residues 37-45; SEQ ID NO:241), or VLDFAPPGAS (human WT1 residues 37-46; SEQ ID NO:411). Further immunogenic portions are provided herein, and others may generally be identified using well known techniques, such as those summarized in Paul, Fundamental Immunology, 3rd ed., 243-247 (Raven Press, 1993) and references cited therein. Representative techniques for identifying immunogenic portions include screening polypeptides for the ability to react with antigen-specific antisera and/or T-cell lines or clones. An immunogenic portion of a native WT1 polypeptide is a portion that reacts with such antisera and/or T-cells at a level that is not substantially less than the reactivity of the full length WT1 (e.g., in an ELISA and/or T-cell reactivity assay). In other words, an immunogenic portion may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988.

Alternatively, immunogenic portions may be identified using computer analysis, such as the Tsites program (see Rothbard and Taylor, EMBO J. 7:93-100, 1988; Deavin et al., Mol. Immunol. 33:145-155, 1996), which searches for peptide motifs that have the potential to elicit Th responses. CTL peptides with motifs appropriate for binding to murine and human class I or class II MHC may be identified according to BIMAS (Parker et al., J. Immunol. 152:163, 1994) and other HLA peptide binding prediction analyses. To confirm immunogenicity, a peptide may be tested using an HLA A2 transgenic mouse model and/or an in vitro stimulation assay using dendritic cells, fibroblasts or peripheral blood cells.

As noted above, a composition may comprise a variant of a native WT1 protein. A polypeptide “variant,” as used herein, is a polypeptide that differs from a native polypeptide in one or more substitutions, deletions, additions and/or insertions, such that the immunogenicity of the polypeptide is retained (i.e., the ability of the variant to react with antigen-specific antisera and/or T-cell lines or clones is not substantially diminished relative to the native polypeptide). In other words, the ability of a variant to react with antigen-specific antisera and/or T-cell lines or clones may be enhanced or unchanged, relative to the native polypeptide, or may be diminished by less than 50%, and preferably less than 20%, relative to the native polypeptide. Such variants may generally be identified by modifying one of the above polypeptide sequences and evaluating the reactivity of the modified polypeptide with antisera and/or T-cells as described herein. It has been found, within the context of the present invention, that a relatively small number of substitutions (e.g., 1 to 3) within an immunogenic portion of a WT1 polypeptide may serve to enhance the ability of the polypeptide to elicit an immune response. Suitable substitutions may generally be identified by using computer programs, as described above, and the effect confirmed based on the reactivity of the modified polypeptide with antisera and/or T-cells as described herein. Accordingly, within certain preferred embodiments, a WT1 polypeptide comprises a variant in which 1 to 3 amino acid resides within an immunogenic portion are substituted such that the ability to react with antigen-specific antisera and/or T-cell lines or clones is statistically greater than that for the unmodified polypeptide. Such substitutions are preferably located within an MHC binding site of the polypeptide, which may be identified as described above. Preferred substitutions allow increased binding to MHC class I or class II molecules.

Certain variants contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. Variants may also (or alternatively) be modified by, for example, the deletion or addition of amino acids that have minimal influence on the immunogenicity, secondary structure and hydropathic nature of the polypeptide.

In a preferred embodiment, a variant polypeptide of the WT1 N-terminus (amino acids 1-249) is constructed, wherein the variant polypeptide is capable of binding to an antibody that recognizes full-length WT1 and/or WT1 N-terminus polypeptide. A non-limiting example of an antibody is anti WT1 antibody WT180 (Santa Cruz Biotechnology, Inc., Santa Cruz, Calif.).

As noted above, WT1 polypeptides may be conjugated to a signal (or leader) sequence at the N-terminal end of the protein which co-translationally or post-translationally directs transfer of the protein. A polypeptide may also, or alternatively, be conjugated to a linker or other sequence for ease of synthesis, purification or identification of the polypeptide (e g., poly-His), or to enhance binding of the polypeptide to a solid support. For example, a polypeptide may be conjugated to an immunoglobulin Fc region.

WT1 polypeptides may be prepared using any of a variety of well known techniques. Recombinant polypeptides encoded by a WT1 polynucleotide as described herein may be readily prepared from the polynucleotide. In general, any of a variety of expression vectors known to those of ordinary skill in the art may be employed to express recombinant WT1 polypeptides. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. The concentrate may then be applied to a suitable purification matrix such as an affinity matrix or an ion exchange resin. Finally, one or more reverse phase HPLC steps can be employed to further purify a recombinant polypeptide. Such techniques may be used to prepare native polypeptides or variants thereof. For example, polynucleotides that encode a variant of a native polypeptide may generally be prepared using standard mutagenesis techniques, such as oligonucleotide-directed site-specific mutagenesis, and sections of the DNA sequence may be removed to permit preparation of truncated polypeptides.

Certain portions and other variants may also be generated by synthetic means, using techniques well known to those of ordinary skill in the art. For example, polypeptides having fewer than about 500 amino acids, preferably fewer than about 100 amino acids, and more preferably fewer than about 50 amino acids, may be synthesized. Polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc. (Foster City, Calif.), and may be operated according to the manufacturer's instructions.

In general, polypeptides and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Within further aspects, the present invention provides mimetics of WT1 polypeptides. Such mimetics may comprise amino acids linked to one or more amino acid mimetics (i.e., one or more amino acids within the WT1 protein may be replaced by an amino acid mimetic) or may be entirely nonpeptide mimetics. An amino acid mimetic is a compound that is conformationally similar to an amino acid such that it can be substituted for an amino acid within a WT1 polypeptide without substantially diminishing the ability to react with antigen-specific antisera and/or T cell lines or clones. A nonpeptide mimetic is a compound that does not contain amino acids, and that has an overall conformation that is similar to a WT1 polypeptide such that the ability of the mimetic to react with WT1-specific antisera and/or T cell lines or clones is not substantially diminished relative to the ability of a WT1 polypeptide. Such mimetics may be designed based on standard techniques (e.g., nuclear magnetic resonance and computational techniques) that evaluate the three dimensional structure of a peptide sequence. Mimetics may be designed where one or more of the side chain functionalities of the WT1 polypeptide are replaced by groups that do not necessarily have the same size or volume, but have similar chemical and/or physical properties which produce similar biological responses. It should be understood that, within embodiments described herein, a mimetic may be substituted for a WT1 polypeptide.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

The invention provides truncated forms of WT1 polypeptides that can be recombinantly expressed in E. coli without the addition of a fusion partner. Examples of these truncated forms are shown in SEQ ID NOs:342-346, and are encoded by polynucleotides shown in SEQ ID NOs:337-341. In variations of these truncations, the first 76 amino acids of WT1 can be fused to the C-terminus of the protein, creating a recombinant protein that is easier to express in E. coli. Other hosts in addition to E. coli can also be used, such as, for example, B. megaterium. The protein can further be prepared without a histidine tag.

In other embodiments, different subunits can be made and fused together in an order which differs from that of native WT1. In addition, fusions can be made with, for example, Ra12. Exemplary fusion proteins are shown in SEQ ID NOs: 332-336 and can be encoded by polynucleotides shown in SEQ ID NOs: 327-331.

WT1 Polynucleotides

Any polynucleotide that encodes a WT1 polypeptide as described herein is a WT1 polynucleotide encompassed by the present invention. Such polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

WT1 polynucleotides may encode a native WT1 protein, or may encode a variant of WT1 as described herein. Polynucleotide variants may contain one or more substitutions, additions, deletions and/or insertions such that the immunogenicity of the encoded polypeptide is not diminished, relative to a native WT1 protein. The effect on the immunogenicity of the encoded polypeptide may generally be assessed as described herein. Preferred variants contain nucleotide substitutions, deletions, insertions and/or additions at no more than 20%, preferably at no more than 10%, of the nucleotide positions that encode an immunogenic portion of a native WT1 sequence. Certain variants are substantially homologous to a native gene, or a portion thereof. Such polynucleotide variants are capable of hybridizing under moderately stringent conditions to a naturally occurring DNA sequence encoding a WT1 polypeptide (or a complementary sequence). Suitable moderately stringent conditions include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2× SSC containing 0.1% SDS). Such hybridizing DNA sequences are also within the scope of this invention.

It will be appreciated by those of ordinary skill in the art that, as a result of the degeneracy of the genetic code, there are many nucleotide sequences that encode a WT1 polypeptide. Some of these polynucleotides bear minimal homology to the nucleotide sequence of any native gene. Nonetheless, polynucleotides that vary due to differences in codon usage are specifically contemplated by the present invention.

Once an immunogenic portion of WT1 is identified, as described above, a WT1 polynucleotide may be prepared using any of a variety of techniques. For example, a WT1 polynucleotide may be amplified from cDNA prepared from cells that express WT1. Such polynucleotides may be amplified via polymerase chain reaction (PCR). For this approach, sequence-specific primers may be designed based on the sequence of the immunogenic portion and may be purchased or synthesized. For example, suitable primers for PCR amplification of a human WT1 gene include: first step-P118: 1434-1414: 5′ GAG AGT CAG ACT TGA AAG CAGT 3′ (SEQ ID NO:5) and P135: 5′ CTG AGC CTC AGC AAA TGG GC 3′ (SEQ ID NO:6); second step-P136: 5′ GAG CAT GCA TGG GCT CCG ACG TGC GGG 3′ (SEQ ID NO:7) and P137: 5′ GGG GTA CCC ACT GAA CGG TCC CCG A 3′ (SEQ ID NO:8). Primers for PCR amplification of a mouse WT1 gene include: first step-P138: 5′ TCC GAG CCG CAC CTC ATG 3′ (SEQ ID NO:9) and P139: 5′ GCC TGG GAT GCT GGA CTG 3′ (SEQ ID NO:10), second step-P140: 5′ GAG CAT GCG ATG GGT TCC GAC GTG CGG 3′ (SEQ ID NO:11) and P141: 5′ GGG GTA CCT CAA AGC GCC ACG TGG AGT TT 3′ (SEQ ID NO:12).

An amplified portion may then be used to isolate a full length gene from a human genomic DNA library or from a suitable cDNA library, using well known techniques. Alternatively, a full length gene can be constructed from multiple PCR fragments. WT1 polynucleotides may also be prepared by synthesizing oligonucleotide components, and ligating components together to generate the complete polynucleotide.

WT1 polynucleotides may also be synthesized by any method known in the art, including chemical synthesis (e.g., solid phase phosphoramidite chemical synthesis). Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences encoding a WT1 polypeptide, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo (e.g., by transfecting antigen-presenting cells such as dendritic cells with a cDNA construct encoding a WT1 polypeptide, and administering the transfected cells to the patient).

Polynucleotides that encode a WT1 polypeptide may generally be used for production of the polypeptide, in vitro or in vivo. WT1 polynucleotides that are complementary to a coding sequence (i.e., antisense polynucleotides) may also be used as a probe or to inhibit WT1 expression. cDNA constructs that can be transcribed into antisense RNA may also be introduced into cells of tissues to facilitate the production of antisense RNA.

Any polynucleotide may be further modified to increase stability in vivo. Possible modifications include, but are not limited to, the addition of flanking sequences at the 5′ and/or 3′ ends; the use of phosphorothioate or 2′O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art. cDNA constructs within such a vector may be used, for example, to transfect human or animal cell lines for use in establishing WT1 positive tumor models which may be used to perform tumor protection and adoptive immunotherapy experiments to demonstrate tumor or leukemia-growth inhibition or lysis of such cells.

Other therapeutic formulations for polynucleotides include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Antibodies and Fragments Thereof

The present invention further provides binding agents, such as antibodies and antigen-binding fragments thereof, that specifically bind to a WT1 polypeptide. As used herein, an agent is said to “specifically bind” to a WT1 polypeptide if it reacts at a detectable level (within, for example, an ELISA) with a WT1 polypeptide, and does not react detectably with unrelated proteins under similar conditions. As used herein, “binding” refers to a noncovalent association between two separate molecules such that a “complex” is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 103 L/mol. The binding constant maybe determined using methods well known in the art.

Any agent that satisfies the above requirements may be a binding agent. In a preferred embodiment, a binding agent is an antibody or an antigen-binding fragment thereof. Certain antibodies are commercially available from, for example, Santa Cruz Biotechnology (Santa Cruz, Calif.). Alternatively, antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fe fragments. The Fab and Fc fragments may be separated by affinity chromatography on protein A bead columns.

Monoclonal antibodies and fragments thereof may be coupled to one or more therapeutic agents. Suitable agents in this regard include radioactive tracers and chemotherapeutic agents, which may be used, for example, to purge autologous bone marrow in vitro). Representative therapeutic agents include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein. For diagnostic purposes, coupling of radioactive agents may be used to facilitate tracing of metastases or to determine the location of WT1 -positive tumors.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used. A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

Also provided herein are anti-idiotypic antibodies that mimic an immunogenic portion of WT1. Such antibodies may be raised against an antibody, or antigen-binding fragment thereof, that specifically binds to an immunogenic portion of WT1, using well known techniques. Anti-idiotypic antibodies that mimic an immunogenic portion of WT1 are those antibodies that bind to an antibody, or antigen-binding fragment thereof, that specifically binds to an immunogenic portion of WT1, as described herein.

T Cells

Immunotherapeutic compositions may also, or alternatively, comprise T cells specific for WT1. Such cells may generally be prepared in vitro or ex vivo, using standard procedures. For example, T cells may be present within (or isolated from) bone marrow, peripheral blood or a fraction of bone marrow or peripheral blood of a mammal, such as a patient, using a commercially available cell separation system, such as the CEPRATE™ system, available from CellPro Inc., Bothell Wash. (see also U.S. Pat. Nos. 5,240,856; 5,215,926; WO 89/06280; WO 91/16116 and WO 92/07243). Alternatively, T cells may be derived from related or unrelated humans, non-human animals, cell lines or cultures.

T cells may be stimulated with WT1 polypeptide, polynucleotide encoding a WT1 polypeptide and/or an antigen presenting cell (APC) that expresses a WT1 polypeptide. Such stimulation is performed under conditions and for a time sufficient to permit the generation of T cells that are specific for the WT1 polypeptide. Preferably, a WT1 polypeptide or polynucleotide is present within a delivery vehicle, such as a microsphere, to facilitate the generation of antigen-specific T cells. Briefly, T cells, which may be isolated from a patient or a related or unrelated donor by routine techniques (such as by Ficoll/Hypaque density gradient centrifugation of peripheral blood lymphocytes), are incubated with WT1 polypeptide. For example, T cells may be incubated in vitro for 2-9 days (typically 4 days) at 37° C. with WT1 polypeptide (e.g., 5 to 25 μg/ml) or cells synthesizing a comparable amount of WT1 polypeptide. It may be desirable to incubate a separate aliquot of a T cell sample in the absence of WT1 polypeptide to serve as a control.

T cells are considered to be specific for a WT1 polypeptide if the T cells kill target cells coated with a WT1 polypeptide or expressing a gene encoding such a polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Other ways to detect T cell proliferation include measuring increases in interleukin-2 (IL-2) production, Ca²⁺ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a WT1 polypeptide may be quantified. Contact with a WT1 polypeptide (200 ng/ml-100 μg/ml, preferably 100 ng/ml-25 μg/ml) for 3-7 days should result in at least a two fold increase in proliferation of the T cells and/or contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998). WT1 specific T cells may be expanded using standard techniques. Within preferred embodiments, the T cells are derived from a patient or a related or unrelated donor and are administered to the patient following stimulation and expansion.

T cells that have been activated in response to a WT1 polypeptide, polynucleotide or WT1-expressing APC may be CD4⁺ and/or CD8⁺. Specific activation of CD4⁺ or CD8⁺ T cells may be detected in a variety of ways. Methods for detecting specific T cell activation include detecting the proliferation of T cells, the production of cytokines (e.g., lymphokines), or the generation of cytolytic activity (i.e., generation of cytotoxic T cells specific for WT1). For CD4⁺ T cells, a preferred method for detecting specific T cell activation is the detection of the proliferation of T cells. For CD8⁺ T cells, a preferred method for detecting specific T cell activation is the detection of the generation of cytolytic activity.

For therapeutic purposes, CD4⁺ or CD8⁺ T cells that proliferate in response to the WT1 polypeptide, polynucleotide or APC can be expanded in number either in vitro or in vivo. Proliferation of such T cells in vitro may be accomplished in a variety of ways. For example, the T cells can be re-exposed to WT1 polypeptide, with or without the addition of T cell growth factors, such as interleukin-2, and/or stimulator cells that synthesize a WT1 polypeptide. The addition of stimulator cells is preferred where generating CD8⁺ T cell responses. T cells can be grown to large numbers in vitro with retention of specificity in response to intermittent restimulation with WT1 polypeptide. Briefly, for the primary in vitro stimulation (IVS), large numbers of lymphocytes (e.g., greater than 4×10⁷) may be placed in flasks with media containing human serum. WT1 polypeptide (e.g., peptide at 10 μg/ml) may be added directly, along with tetanus toxoid (e.g., 5 μg/ml). The flasks may then be incubated (e.g., 37° C. for 7 days). For a second IVS, T cells are then harvested and placed in new flasks with 2-3×10⁷ irradiated peripheral blood mononuclear cells. WT1 polypeptide (e.g., 10 μg/ml) is added directly. The flasks are incubated at 37° C. for 7 days. On day 2 and day 4 after the second IVS, 2-5 units of interleukin-2 (IL-2) may be added. For a third IVS, the T cells may be placed in wells and stimulated with the individual's own EBV transformed B cells coated with the peptide. IL-2 may be added on days 2 and 4 of each cycle. As soon as the cells are shown to be specific cytotoxic T cells, they may be expanded using a 10 day stimulation cycle with higher IL-2 (20 units) on days 2, 4 and 6.

Alternatively, one or more T cells that proliferate in the presence of WT1 polypeptide can be expanded in number by cloning. Methods for cloning cells are well known in the art, and include limiting dilution. Responder T cells may be purified from the peripheral blood of sensitized patients by density gradient centrifugation and sheep red cell rosetting and established in culture by stimulating with the nominal antigen in the presence of irradiated autologous filler cells. In order to generate CD4⁺ T cell lines, WT1 polypeptide is used as the antigenic stimulus and autologous peripheral blood lymphocytes (PBL) or lymphoblastoid cell lines (LCL) immortalized by infection with Epstein Barr virus are used as antigen presenting cells. In order to generate CD8⁺ T cell lines, autologous antigen-presenting cells transfected with an expression vector which produces WT1 polypeptide may be used as stimulator cells. Established T cell lines may be cloned 2-4 days following antigen stimulation by plating stimulated T cells at a frequency of 0.5 cells per well in 96-well flat-bottom plates with 1×10⁶ irradiated PBL or LCL cells and recombinant interleukin-2 (rIL2) (50 U/ml). Wells with established clonal growth may be identified at approximately 2-3 weeks after initial plating and restimulated with appropriate antigen in the presence of autologous antigen-presenting cells, then subsequently expanded by the addition of low doses of rIL2 (10 U/ml) 2-3 days following antigen stimulation. T cell clones may be maintained in 24-well plates by periodic restimulation with antigen and rIL2 approximately every two weeks.

Within certain embodiments, allogeneic T-cells may be primed (i.e., sensitized to WT1) in vivo and/or in vitro. Such priming may be achieved by contacting T cells with a WT1 polypeptide, a polynucleotide encoding such a polypeptide or a cell producing such a polypeptide under conditions and for a time sufficient to permit the priming of T cells. In general, T cells are considered to be primed if, for example, contact with a WT1 polypeptide results in proliferation and/or activation of the T cells, as measured by standard proliferation, chromium release and/or cytokine release assays as described herein. A stimulation index of more than two fold increase in proliferation or lysis, and more than three fold increase in the level of cytokine, compared to negative controls, indicates T-cell specificity. Cells primed in vitro may be employed, for example, within a bone marrow transplantation or as donor lymphocyte infusion.

T cells specific for WT1 can kill cells that express WT1 protein. Introduction of genes encoding T-cell receptor (TCR) chains for WT1 are used as a means to quantitatively and qualitatively improve responses to WT1 bearing leukemia and cancer cells. Vaccines to increase the number of T cells that can react to WT1 positive cells are one method of targeting WT1 bearing cells. T cell therapy with T cells specific for WT1 is another method. An alternative method is to introduce the TCR chains specific for WT1 into T cells or other cells with lytic potential. In a suitable embodiment, the TCR alpha and beta chains are cloned out from a WT1 specific T cell line and used for adoptive T cell therapy, such as described in WO96/30516, incorporated herein by reference.

Pharmaceutical Compositions and Vaccines

Within certain aspects, polypeptides, polynucleotides, antibodies and/or T cells may be incorporated into pharmaceutical compositions or vaccines. Alternatively, a pharmaceutical composition may comprise an antigen-presenting cell (e.g., a dendritic cell) transfected with a WT1 polynucleotide such that the antigen presenting cell expresses a WT1 polypeptide. Pharmaceutical compositions comprise one or more such compounds or cells and a physiologically acceptable carrier or excipient. Certain vaccines may comprise one or more such compounds or cells and a non-specific immune response enhancer, such as an adjuvant or a liposome (into which the compound is incorporated). Pharmaceutical compositions and vaccines may additionally contain a delivery system, such as biodegradable microspheres which are disclosed, for example, in U.S. Pat. Nos. 4,897,268 and 5,075,109. Pharmaceutical compositions and vaccines within the scope of the present invention may also contain other compounds, which may be biologically active or inactive.

Within certain embodiments, pharmaceutical compositions and vaccines are designed to elicit T cell responses specific for a WT1 polypeptide in a patient, such as a human. In general, T cell responses may be favored through the use of relatively short polypeptides (e.g., comprising less than 23 consecutive amino acid residues of a native WT1 polypeptide, preferably 4-16 consecutive residues, more preferably 8-16 consecutive residues and still more preferably 8-10 consecutive residues. Alternatively, or in addition, a vaccine may comprise a non-specific immune response enhancer that preferentially enhances a T cell response. In other words, the immune response enhancer may enhance the level of a T cell response to a WT1 polypeptide by an amount that is proportionally greater than the amount by which an antibody response is enhanced. For example, when compared to a standard oil based adjuvant, such as CFA, an immune response enhancer that preferentially enhances a T cell response may enhance a proliferative T cell response by at least two fold, a lytic response by at least 10%, and/or T cell activation by at least two fold compared to WT1-megative control cell lines, while not detectably enhancing an antibody response. The amount by which a T cell or antibody response to a WT1 polypeptide is enhanced may generally be determined using any representative technique known in the art, such as the techniques provided herein.

A pharmaceutical composition or vaccine may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacterial and viral expression systems and mammalian expression systems. Appropriate nucleic acid expression systems contain the necessary DNA, cDNA or RNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

As noted above, a pharmaceutical composition or vaccine may comprise an antigen-presenting cell that expresses a WT1 polypeptide. For therapeutic purposes, as described herein, the antigen presenting cell is preferably an autologous dendritic cell. Such cells may be prepared and transfected using standard techniques, such as those described by Reeves et al., Cancer Res. 56:5672-5677, 1996; Tuting et al., J. Immunol. 160:1139-1147, 1998; and Nair et al., Nature Biotechnol. 16:364-369, 1998). Expression of a WT1 polypeptide on the surface of an antigen-presenting cell may be confirmed by in vitro stimulation and standard proliferation as well as chromium release assays, as described herein.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. For certain topical applications, formulation as a cream or lotion, using well known components, is preferred.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology. In one embodiment of the present invention, compositions comprise a buffer comprising one or more sugars including, but not limited to, trehalose, maltose, sucrose, fructose, and glucose, each at a concentration generally between about 1 and 25%, typically between about 7 and 13 %. In a further embodiment, the concentration is between about 8 and about 12%. In yet a further embodiment the concentration is about 10%. In an additional aspect of the present invention, the compositions may comprise ethanolamine; cysteine; or Polysorbate-80, generally at concentrations effective for enhancing the efficacy, stability and/or solubility of the formulation.

Any of a variety of non-specific immune response enhancers, such as adjuvants, may be employed in the vaccines of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable non-specific immune response enhancers include alum-based adjuvants (e.g., Alhydrogel, Rehydragel, aluminum phosphate, Alganunulin, aluminum hydroxide); oil based adjuvants (Freund's adjuvant (FA), Specol, RIBI, TiterMax, Montanide ISA50 or Montanide ISA 720 (Seppic, France); cytokines (e.g., GM-CSF or Flat3-ligand); microspheres; nonionic block copolymer-based adjuvants; dimethyl dioctadecyl ammoniumbromide (DDA) based adjuvants AS-1, AS-2 (Smith Kline Beecham); Ribi Adjuvant system based adjuvants; QS21 (Aquila); saponin based adjuvants (crude saponin, the saponin Quil A ); muramyl dipeptide (MDP) based adjuvants such as SAF (Syntex adjuvant in its microfluidized form (SAF-m)); dimethyl-dioctadecyl ammonium bromide (DDA); human complement based adjuvants m. vaccae and derivatives; immune stimulating complex (iscom) based adjuvants; inactivated toxins; and attenuated infectious agents (such as M. tuberculosis).

Additional illustrative adjuvants for use in the pharmaceutical compositions of the invention include, SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ—A—R, (I):

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

As noted above, within certain embodiments, immune response enhancers are chosen for their ability to preferentially elicit or enhance a T cell response (e.g., CD4⁺ and/or CD8⁺) to a WT1 polypeptide. Such immune response enhancers are well known in the art, and include (but are not limited to) Montanide ISA50, Seppic MONTANIDE ISA 720, cytokines (e.g., GM-CSF, Flat3-ligand), microspheres, dimethyl dioctadecyl ammoniumbromide (DDA) based adjuvants, AS-1 (Smith Kline Beecham), AS-2 (Smith Kline Beecham), Ribi Adjuvant system based adjuvants, QS21 (Aquila), saponin based adjuvants (crude saponin, the saponin Quil A), Syntex adjuvant in its microfluidized form (SAF-m), MV, ddMV (Genesis), immune stimulating complex (iscom) based adjuvants and inactivated toxins.

In another aspect of the present invention, compositions may comprise adjuvants for eliciting a predominantly Th1-type response. Certain preferred adjuvants for eliciting a predominantly Th1-type response include, for example, a combination of monophosphoryl lipid A, preferably 3-de-O-acylated monophosphoryl lipid A, together with an aluminum salt. MPL adjuvants, such as MPL-SE, are available from Corixa Corporation (Seattle, Wash.; see, for example, U.S. Pat. Nos. 4,436,727; 4,877,611; 4,866,034 and 4,912,094, incorporated herein in their entirety). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

The compositions and vaccines described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide, antibody or cell dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Therapy of Malignant Diseases

In further aspects of the present invention, the compositions and vaccines described herein may be used to inhibit the development of malignant diseases (e.g., progressive or metastatic diseases or diseases characterized by small tumor burden such as minimal residual disease). In general, such methods may be used to prevent, delay or treat a disease associated with WT1 expression. In other words, therapeutic methods provided herein may be used to treat an existing WT1 -associated disease, or may be used to prevent or delay the onset of such a disease in a patient who is free of disease or who is afflicted with a disease that is not yet associated with WT1 expression.

As used herein, a disease is “associated with WT1 expression” if diseased cells (e.g., tumor cells) at some time during the course of the disease generate detectably higher levels of a WT1 polypeptide than normal cells of the same tissue. Association of WT1 expression with a malignant disease does not require that WT1 be present on a tumor. For example, overexpression of WT1 may be involved with initiation of a tumor, but the protein expression may subsequently be lost. Alternatively, a malignant disease that is not characterized by an increase in WT1 expression may, at a later time, progress to a disease that is characterized by increased WT1 expression. Accordingly, any malignant disease in which diseased cells formerly expressed, currently express or are expected to subsequently express increased levels of WT1 is considered to be “associated with WT1 expression.”

Immunotherapy may be performed using any of a variety of techniques, in which compounds or cells provided herein function to remove WT1-expressing cells from a patient. Such removal may take place as a result of enhancing or inducing an immune response in a patient specific for WT1 or a cell expressing WT1. Alternatively, WT1-expressing cells may be removed ex vivo (e.g., by treatment of autologous bone marrow, peripheral blood or a fraction of bone marrow or peripheral blood). Fractions of bone marrow or peripheral blood may be obtained using any standard technique in the art.

Within such methods, pharmaceutical compositions and vaccines may be administered to a patient. As used herein, a “patient” refers to any warm-blooded animal, preferably a human. A patient may or may not be afflicted with a malignant disease. Accordingly, the above pharmaceutical compositions and vaccines may be used to prevent the onset of a disease (i.e., prophylactically) or to treat a patient afflicted with a disease (e.g., to prevent or delay progression and/or metastasis of an existing disease). A patient afflicted with a disease may have a minimal residual disease (e.g., a low tumor burden in a leukemia patient in complete or partial remission or a cancer patient following reduction of the tumor burden after surgery radiotherapy and/or chemotherapy). Such a patient may be immunized to inhibit a relapse (i.e., prevent or delay the relapse, or decrease the severity of a relapse). Within certain preferred embodiments, the patient is afflicted with a leukemia (e.g., AML, CML, ALL or childhood ALL), a myelodysplastic syndrome (MDS) or a cancer (e.g., gastrointestinal, lung, thyroid or breast cancer or a melanoma), where the cancer or leukemia is WT1 positive (i.e., reacts detectably with an anti-WT1 antibody, as provided herein or expresses WT1 mRNA at a level detectable by RT-PCR, as described herein) or suffers from an autoimmune disease directed against WT1 -expressing cells.

Other diseases associated with WT1 overexpression include kidney cancer (such as renal cell carcinoma, or Wilms tumor), as described in Satoh F., et al., Pathol. Int. 50(6):458-71(2000), and Campbell C. E. et al., Int. J. Cancer 78(2):182-8 (1998); and mesothelioma, as described in Amin, K. M. et al., Am. J Pathol. 146(2):344-56 (1995). Harada et al. (Mol. Urol. 3(4):357-364 (1999) describe WT1 gene expression in human testicular germ-cell tumors. Nonomura et al. Hinyokika Kiyo 45(8):593-7 (1999) describe molecular staging of testicular cancer using polymerase chain reaction of the testicular cancer-specific genes. Shimizu et al., Int. J Gynecol. Pathol. 19(2):158-63 (2000) describe the immunohistochemical detection of the Wilms' tumor gene (WT1) in epithelial ovarian tumors.

WT1 overexpression was also described in desmoplastic small round cell tumors, by Barnoud, R. et al., Am. J. Surg. Pathol. 24(6):830-6 (2000); and Pathol. Res. Pract. 194(10):693-700 (1998). WT1 overexpression in glioblastoma and other cancer was described by Menssen, H. D. et al., J. Cancer Res. Clin. Oncol. 126(4):226-32 (2000), “Wilms' tumor gene (WT1) expression in lung cancer, colon cancer and glioblastoma cell lines compared to freshly isolated tumor specimens.” Other diseases showing WT1 overexpression include EBV associated diseases, such as Burkitt's lymphoma and nasopharyngeal cancer (Spinsanti P. et al., Leuk. Lymphoma 38(5-6):611-9 (2000), “Wilms' tumor gene expression by normal and malignant human B lymphocytes.”

In Leukemia 14(9):1634-4 (2000), Pan et al., describe in vitro IL-12 treatment of peripheral blood mononuclear cells from patients with leukemia or myelodysplastic syndromes, and reported an increase in cytotoxicity and reduction in WT1 gene expression. In Leukemia 13(6):891-900 (1999), Patmasiriwat et al. reported WT1 and GATA1 expression in myelodysplastic syndrome and acute leukemia. In Leukemia 13(3):393-9 (1999), Tamaki et al. reported that the Wilms' tumor gene WT1 is a good marker for diagnosis of disease progression of myelodysplastic syndromes. Expression of the Wilms' tumor gene WT1 in solid tumors, and its involvement in tumor cell growth, was discussed in relation to gastric cancer, colon cancer, lung cancer, breast cancer cell lines, germ cell tumor cell line, ovarian cancer, the uterine cancer, thyroid cancer cell line, hepatocellular carcinoma, in Oji et al., Jpn. J. Cancer Res. 90(2):194-204 (1999).

The compositions provided herein may be used alone or in combination with conventional therapeutic regimens such as surgery, irradiation, chemotherapy and/or bone marrow transplantation (autologous, syngeneic, allogeneic or unrelated). As discussed in greater detail below, binding agents and T cells as provided herein may be used for purging of autologous stem cells. Such purging may be beneficial prior to, for example, bone marrow transplantation or transfusion of blood or components thereof. Binding agents, T cells, antigen presenting cells (APC) and compositions provided herein may further be used for expanding and stimulating (or priming) autologous, allogeneic, syngeneic or unrelated WT1-specific T-cells in vitro and/or in vivo. Such WT1-specific T cells may be used, for example, within donor lymphocyte infusions.

Routes and frequency of administration, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. In some tumors, pharmaceutical compositions or vaccines may be administered locally (by, for example, rectocoloscopy, gastroscopy, videoendoscopy, angiography or other methods known in the art). Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response that is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent complete or partial remissions, or longer disease-free and/or overall survival) in vaccinated patients as compared to non-vaccinated patients. In general, for pharmaceutical compositions and vaccines comprising one or more polypeptides, the amount of each polypeptide present in a dose ranges from about 100 μg to 5 mg. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent complete or partial remissions, or longer disease-free and/or overall survival) in treated patients as compared to non-treated patients. Increases in preexisting immune responses to WT1 generally correlate with an improved clinical outcome. Such immune responses may generally be evaluated using standard proliferation, cytotoxicity or cytokine assays, which may be performed using samples obtained from a patient before and after treatment.

Within further aspects, methods for inhibiting the development of a malignant disease associated with WT1 expression involve the administration of autologous T cells that have been activated in response to a WT1 polypeptide or WT1-expressing APC, as described above. Such T cells may be CD4⁺ and/or CD8⁺, and may be proliferated as described above. The T cells may be administered to the individual in an amount effective to inhibit the development of a malignant disease. Typically, about 1×10⁹ to 1×10¹¹ T cells/M² are administered intravenously, intracavitary or in the bed of a resected tumor. It will be evident to those skilled in the art that the number of cells and the frequency of administration will be dependent upon the response of the patient.

Within certain embodiments, T cells may be stimulated prior to an autologous bone marrow transplantation. Such stimulation may take place in vivo or in vitro. For in vitro stimulation, bone marrow and/or peripheral blood (or a fraction of bone marrow or peripheral blood) obtained from a patient may be contacted with a WT1 polypeptide, a polynucleotide encoding a WT1 polypeptide and/or an APC that expresses a WT1 polypeptide under conditions and for a time sufficient to permit the stimulation of T cells as described above. Bone marrow, peripheral blood stem cells and/or WT1-specific T cells may then be administered to a patient using standard techniques.

Within related embodiments, T cells of a related or unrelated donor may be stimulated prior to a syngeneic or allogeneic (related or unrelated) bone marrow transplantation. Such stimulation may take place in vivo or in vitro. For in vitro stimulation, bone marrow and/or peripheral blood (or a fraction of bone marrow or peripheral blood) obtained from a related or unrelated donor may be contacted with a WT1 polypeptide, WT1 polynucleotide and/or APC that expresses a WT1 polypeptide under conditions and for a time sufficient to permit the stimulation of T cells as described above. Bone marrow, peripheral blood stem cells and/or WT1-specific T cells may then be administered to a patient using standard techniques.

Within other embodiments, WT1 -specific T cells as described herein may be used to remove cells expressing WT1 from autologous bone marrow, peripheral blood or a fraction of bone marrow or peripheral blood (e.g., CD34⁺ enriched peripheral blood (PB) prior to administration to a patient). Such methods may be performed by contacting bone marrow or PB with such T cells under conditions and for a time sufficient to permit the reduction of WT1 expressing cells to less than 10%, preferably less than 5% and more preferably less than 1%, of the total number of myeloid or lymphatic cells in the bone marrow or peripheral blood. The extent to which such cells have been removed may be readily determined by standard methods such as, for example, qualitative and quantitative PCR analysis, morphology, immunohistochemistry and FACS analysis. Bone marrow or PB (or a fraction thereof) may then be administered to a patient using standard techniques.

Diagnostic Methods

The present invention further provides methods for detecting a malignant disease associated with WT1 expression, and for monitoring the effectiveness of an immunization or therapy for such a disease. Such methods are based on the discovery, within the present invention, that an immune response specific for WT1 protein can be detected in patients afflicted with such diseases, and that methods which enhance such immune responses may provide a preventive or therapeutic benefit.

To determine the presence or absence of a malignant disease associated with WT1 expression, a patient may be tested for the level of T cells specific for WT1. Within certain methods, a biological sample comprising CD4⁺ and/or CD8⁺ T cells isolated from a patient is incubated with a WT1 polypeptide, a polynucleotide encoding a WT1 polypeptide and/or an APC that expresses a WT1 polypeptide, and the presence or absence of specific activation of the T cells is detected, as described herein. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with WT1 polypeptide (e.g., 5-25 μg/ml). It may be desirable to incubate another aliquot of a T cell sample in the absence of WT1 polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a malignant disease associated with WT1 expression. Further correlation may be made, using methods well known in the art, between the level of proliferation and/or cytolytic activity and the predicted response to therapy. In particular, patients that display a higher antibody, proliferative and/or lytic response may be expected to show a greater response to therapy.

Within other methods, a biological sample obtained from a patient is tested for the level of antibody specific for WT1. The biological sample is incubated with a WT1 polypeptide, a polynucleotide encoding a WT1 polypeptide and/or an APC that expresses a WT1 polypeptide under conditions and for a time sufficient to allow immunocomplexes to form. Immunocomplexes formed between the WT1 polypeptide and antibodies in the biological sample that specifically bind to the WT1 polypeptide are then detected. A biological sample for use within such methods may be any sample obtained from a patient that would be expected to contain antibodies. Suitable biological samples include blood, sera, ascites, bone marrow, pleural effusion, and cerebrospinal fluid.

The biological sample is incubated with the WT1 polypeptide in a reaction mixture under conditions and for a time sufficient to permit immunocomplexes to form between the polypeptide and antibodies specific for WT1. For example, a biological sample and WT1 polypeptide may be incubated at 4° C. for 24-48 hours.

Following the incubation, the reaction mixture is tested for the presence of immunocomplexes. Detection of immunocomplexes formed between the WT1 polypeptide and antibodies present in the biological sample may be accomplished by a variety of known techniques, such as radioimmunoassays (RIA) and enzyme linked immunosorbent assays (ELISA). Suitable assays are well known in the art and are amply described in the scientific and patent literature (e.g., Harlow and Lane, Antibodies: A Laboratory Manual, Cold Spring Harbor Laboratory, 1988). Assays that may be used include, but are not limited to, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh, 1970); the “western blot” method of Gordon et al. (U.S. Pat. No. 4,452,901); immunoprecipitation of labeled ligand (Brown et al., J. Biol. Chem. 255:4980-4983, 1980); enzyme-linked immunosorbent assays as described by, for example, Raines and Ross (J. Biol. Chem. 257:5154-5160, 1982); immunocytochemical techniques, including the use of fluorochromes (Brooks et al., Clin. Exp. Immunol. 39: 477, 1980); and neutralization of activity (Bowen-Pope et al., Proc. Natl. Acad. Sci. USA 81:2396-2400, 1984). Other immunoassays include, but are not limited to, those described in U.S. Pat. Nos.: 3,817,827; 3,850,752; 3,901,654; 3,935,074; 3,984,533; 3,996,345; 4,034,074; and 4,098,876.

For detection purposes, WT1 polypeptide may either be labeled or unlabeled. Unlabeled WT1 polypeptide may be used in agglutination assays or in combination with labeled detection reagents that bind to the immunocomplexes (e.g., anti-immunoglobulin, protein G, protein A or a lectin and secondary antibodies, or antigen-binding fragments thereof, capable of binding to the antibodies that specifically bind to the WT1 polypeptide). If the WT1 polypeptide is labeled, the reporter group may be any suitable reporter group known in the art, including radioisotopes, fluorescent groups, luminescent groups, enzymes, biotin and dye particles.

Within certain assays, unlabeled WT1 polypeptide is immobilized on a solid support. The solid support may be any material known to those of ordinary skill in the art to which the polypeptide may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The polypeptide may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the antigen and functional groups on the support or may be a linkage by way of a cross-linking agent). Immobilization by adsorption to a well in a microtiter plate or to a membrane is preferred. In such cases, adsorption may be achieved by contacting the WT1 polypeptide, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of polypeptide ranging from about 10 ng to about 10 μg, and preferably about 100 ng to about 1 μg, is sufficient to immobilize an adequate amount of polypeptide.

Following immobilization, the remaining protein binding sites on the support are typically blocked. Any suitable blocking agent known to those of ordinary skill in the art, such as bovine serum albumin, Tween 20™ (Sigma Chemical Co., St. Louis, Mo.), heat-inactivated normal goat serum (NGS), or BLOTTO (buffered solution of nonfat dry milk which also contains a preservative, salts, and an antifoaming agent). The support is then incubated with a biological sample suspected of containing specific antibody. The sample can be applied neat, or, more often, it can be diluted, usually in a buffered solution which contains a small amount (0.1%-5.0% by weight) of protein, such as BSA, NGS, or BLOTTO. In general, an appropriate contact time (i.e., incubation time) is a period of time that is sufficient to detect the presence of antibody that specifically binds WT1 within a sample containing such an antibody. Preferably, the contact time is sufficient to achieve a level of binding that is at least about 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. A detection reagent that binds to the immunocomplexes and that comprises a reporter group may then be added. The detection reagent is incubated with the immunocomplex for an amount of time sufficient to detect the bound antibody. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups (e.g., horseradish peroxidase, beta-galactosidase, alkaline phosphatase and glucose oxidase) may generally be detected by the addition of substrate (generally for a specific period of time), followed by spectroscopic or other analysis of the reaction products. Regardless of the specific method employed, a level of bound detection reagent that is at least two fold greater than background (i.e., the level observed for a biological sample obtained from a disease-free individual) indicates the presence of a malignant disease associated with WT1 expression.

In general, methods for monitoring the effectiveness of an immunization or therapy involve monitoring changes in the level of antibodies or T cells specific for WT1 in the patient. Methods in which antibody levels are monitored may comprise the steps of: (a) incubating a first biological sample, obtained from a patient prior to a therapy or immunization, with a WT1 polypeptide, wherein the incubation is performed under conditions and for a time sufficient to allow immunocomplexes to form; (b) detecting immunocomplexes formed between the WT1 polypeptide and antibodies in the biological sample that specifically bind to the WT1 polypeptide; (c) repeating steps (a) and (b) using a second biological sample taken from the patient following therapy or immunization; and (d) comparing the number of immunocomplexes detected in the first and second biological samples. Alternatively, a polynucleotide encoding a WT1 polypeptide, or an APC expressing a WT1 polypeptide may be employed in place of the WT1 polypeptide. Within such methods, immunocomplexes between the WT1 polypeptide encoded by the polynucleotide, or expressed by the APC, and antibodies in the biological sample are detected.

Methods in which T cell activation and/or the number of WT1 specific precursors are monitored may comprise the steps of: (a) incubating a first biological sample comprising CD4+ and/or CD8+ cells (e.g., bone marrow, peripheral blood or a fraction thereof), obtained from a patient prior to a therapy or immunization, with a WT1 polypeptide, wherein the incubation is performed under conditions and for a time sufficient to allow specific activation, proliferation and/or lysis of T cells; (b) detecting an amount of activation, proliferation and/or lysis of the T cells; (c) repeating steps (a) and (b) using a second biological sample comprising CD4+ and/or CD8+ T cells, and taken from the same patient following therapy or immunization; and (d) comparing the amount of activation, proliferation and/or lysis of T cells in the first and second biological samples. Alternatively, a polynucleotide encoding a WT1 polypeptide, or an APC expressing a WT1 polypeptide may be employed in place of the WT1 polypeptide.

A biological sample for use within such methods may be any sample obtained from a patient that would be expected to contain antibodies, CD4+ T cells and/or CD8+ T cells. Suitable biological samples include blood, sera, ascites, bone marrow, pleural effusion and cerebrospinal fluid. A first biological sample may be obtained prior to initiation of therapy or immunization or part way through a therapy or vaccination regime. The second biological sample should be obtained in a similar manner, but at a time following additional therapy or immunization. The second biological sample may be obtained at the completion of, or part way through, therapy or immunization, provided that at least a portion of therapy or immunization takes place between the isolation of the first and second biological samples.

Incubation and detection steps for both samples may generally be performed as described above. A statistically significant increase in the number of immunocomplexes in the second sample relative to the first sample reflects successful therapy or immunization.

The following Examples are offered by way of illustration and not by way of limitation.

### EXAMPLES

### Example 1

### Identification of an Immune Response to WT1 in Patients with Hematological Malignancies

This Example illustrates the identification of an existent immune response in patients with a hematological malignancy.

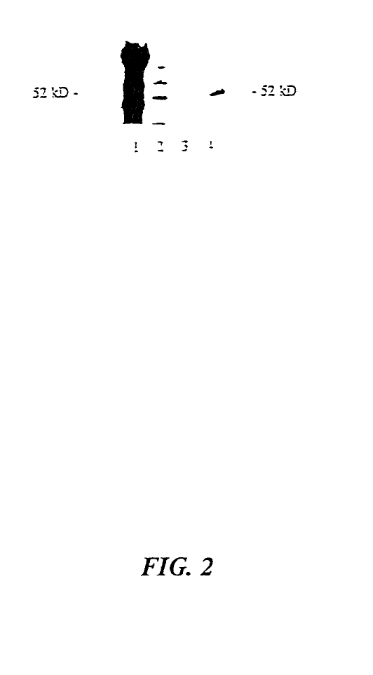

To evaluate the presence of preexisting WT1 specific antibody responses in patients, sera of patients with acute myelogenous leukemia (AML), acute lymphocytic leukemia (ALL), chronic myelogenous leukemia (CML) and severe aplastic anemia were analyzed using Western blot analysis. Sera were tested for the ability to immunoprecipitate WT1 from the human leukemic cell line K562 (American Type Culture Collection, Manassas, Va.). In each case, immunoprecipitates were separated by gel electrophoresis, transferred to membrane and probed with the anti WT1 antibody WT180 (Santa Cruz Biotechnology, Inc., Santa Cruz, Calif.). This Western blot analysis identified potential WT1 specific antibodies in patients with hematological malignancy. A representative Western blot showing the results for a patient with AML is shown in FIG. 2. A 52 kD protein in the immunoprecipitate generated using the patient sera was recognized by the WT1 specific antibody. The 52 kD protein migrated at the same size as the positive control.

Additional studies analyzed the sera of patients with AML and CML for the presence of antibodies to full-length and truncated WT1 proteins. cDNA constructs representing the human WT1/full-length (aa 1-449), the N-terminus (aa 1-249) (WT1/N-terminus) and C-terminus (aa 267-449) (WT1/C-terminus) region were subcloned into modified pET28 vectors. The WT1/full-length and WT1/N-terminus proteins were expressed as Ra12 fusion proteins. Ra12 is the C-terminal fragment of a secreted Mycobacterium tuberculosis protein, denoted as MTB32B. (Skeiky et al., Infect Immun. 67;3998, 1999). The Ra12-WT1/full-length fusion region was cloned 3′ to a histidine-tag in a histidine-tag modified pET28 vector. The WT1/N-terminus region was subcloned into a modified pET28 vector that has a 5′ histidine-tag followed by the thioredoxin (TRX)-WT1/N-terminus fusion region followed by a 3′ histidine-tag. The WT1/C-terminus coding region was subcloned into a modified pET28 vector without a fusion partner containing only the 5′ and 3′ histidine-tag, followed by a Thrombin and EK site.

BL21 pLysS E. coli (Stratagene, La Jolla, Calif.) were transformed with the three WT1 expression constructs, grown overnight and induced with isopropyl-β-D-thiogalactoside (IPTG). WT1 proteins were purified as follows: Cells were harvested and lysed by incubation in 10 mM Tris, pH 8.0 with Complete Protease Inhibitor Tablets (Boehringer Mannheim Biochemicals, Indianapolis, Ind.) at 37° C. followed by repeated rounds of sonication. Inclusion bodies were washed twice with 10 mM Tris, pH 8.0. Proteins were then purified by metal chelate affinity chromatography over nickel-nitrilotriacetic acid resin (QIAGEN Inc., Valencia, Calif.; Hochuli et al., Biologically Active Molecules:217, 1989) followed by chromatography on a Source Q anion exchange resin (Amersham Pharmacia Biotech, Upsala, Sweden). The identity of the WT1 proteins was confirmed by N-terminal sequencing.

Sera from adult patients with de nova AML or CML were studied for the presence of WT1 specific Ab. Recombinant proteins were adsorbed to TC microwell plates (Nunc, Roskilde, Denmark). Plates were washed with PBS/0.5%Tween 20 and blocked with 1% BSA/PBS/0.1%Tween 20. After washing, serum dilutions were added and incubated overnight at 4° C. Plates were washed and Donkey anti-human IgG-HRP secondary antibody was added (Jackson-Immunochem, West Grove, Pa.) and incubated for 2 h at room temperature. Plates were washed, incubated with TMB Peroxidase substrate solution (Kirkegaard and Perry Laboratories, Mass.), quenched with 1N H₂SO₄, and immediately read (Cyto-Fluor 2350; Millipore, Bedford, Mass.).

For the serological survey, human sera were tested by ELISA over a range of serial dilutions from 1:50 to 1:20,000. A positive reaction was defined as an OD value of a 1:500 diluted serum that exceeded the mean OD value of sera from normal donors (n=96) by three (WT1/full-length, WT1C-terminus) standard deviations. Due to a higher background in normal donors to the WT1/N-terminus protein a positive reaction to WT1/N-terminus was defined as an OD value of 1:500 diluted serum that exceeded the mean OD value of sera from normal donors by four standard deviations. To verify that the patient Ab response was directed against WT1 and not to the Ra12 or TRX fusion part of the protein or possible E. coli contaminant proteins, controls included the Ra12 and TRX protein alone purified in a similar manner. Samples that showed reactivity against the Ra12 and/or TRX proteins were excluded from the analysis.

To evaluate for the presence of immunity to WT1, Ab to recombinant full-length and truncated WT1 proteins in the sera of normal individuals and patients with leukemia were determined. Antibody reactivity was analyzed by ELISA reactivity to WT1/full-length protein, WT1/N-terminus protein and WT1/C-terminus protein.

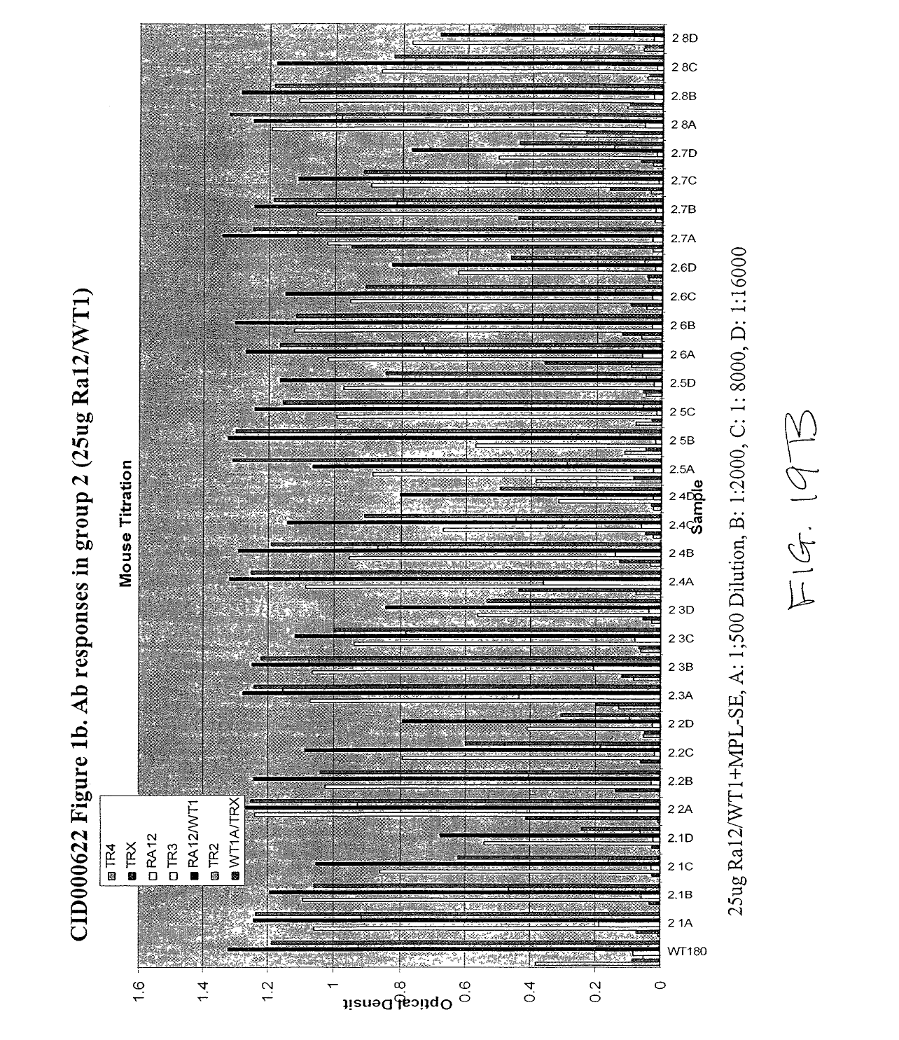

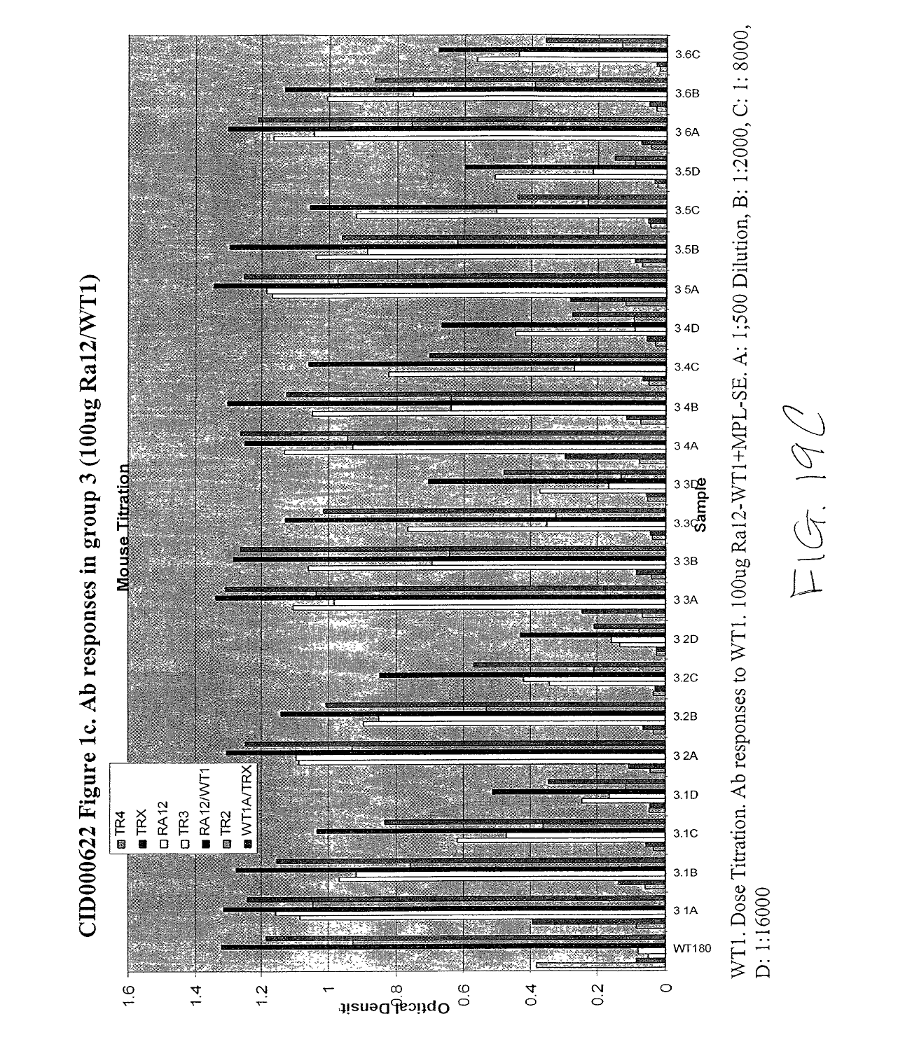

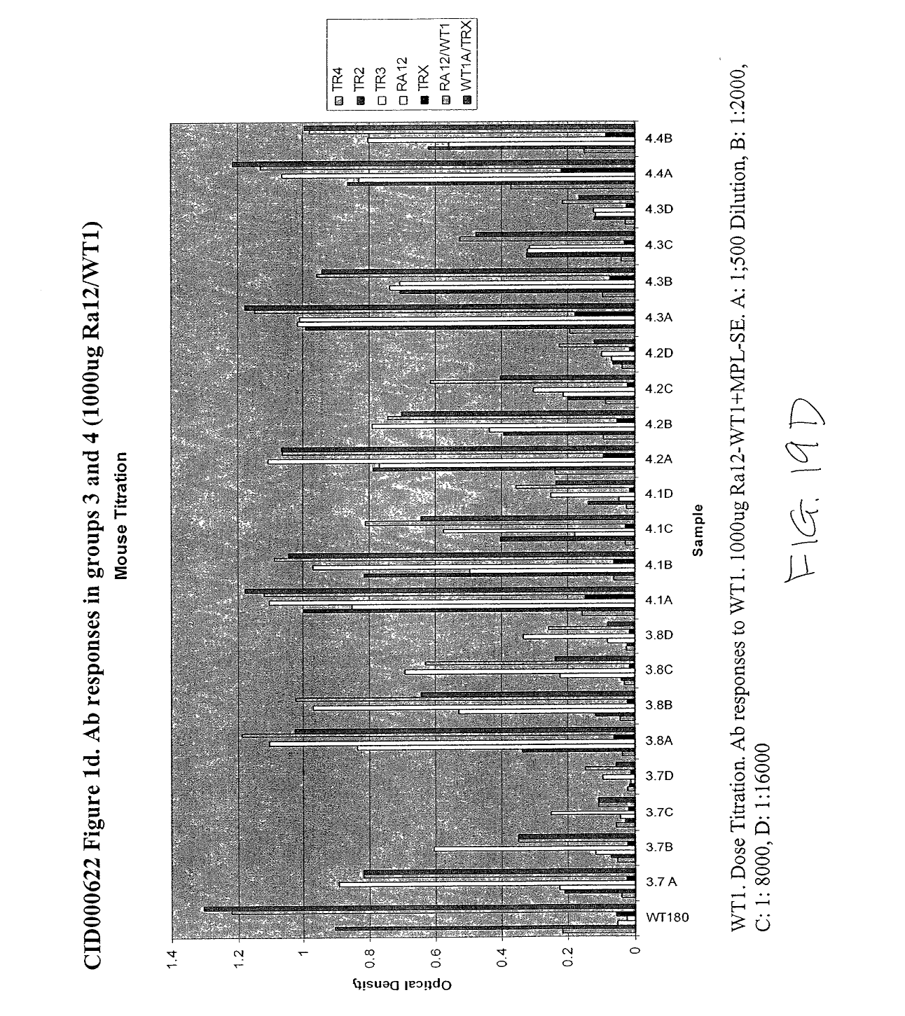

Only 2 of 96 normal donors had serum antibodies reactive with WT1/full-length protein (FIG. 18). One of those individuals had antibody to WT1/N-terminus protein and one had antibody to WT1/C-terminus protein. In contrast, 16 of 63 patients (25%) with AML had serum antibodies reactive with WT1/full-length protein. By marked contrast, only 2 of 63 patients (3%) had reactivity to WT1/C-terminus protein. Fifteen of 81 patients (19%) with CML had serum antibodies reactive with WT1/full-length protein and 12 of 81 patients (15%) had serum antibodies reactive with WT1/N-terminus. Only 3 of 81 patients (3%) had reactivity to WT1/C-terminus protein. (FIGS. 16 and 17.) These data demonstrate that Ab responses to WT1 are detectable in some patients with AML and CML. The greater incidence of antibody in leukemia patients provides strong evidence that immunization to the WT1 protein occurred as a result of patients bearing malignancy that expresses or at some time expressed WT1. Without being limited to a specific theory, it is believed that the observed antibody responses to WT1 most probably result from patients becoming immune to WT1 on their own leukemia cells and provide direct evidence that WT1 can be immunogenic despite being a “self” protein.

The presence of antibody to WT1 strongly implies that concurrent helper T cell responses are also present in the same patients. WT1 is an internal protein. Thus, CTL responses are likely to be the most effective in terms of leukemia therapy and the most toxic arm of immunity. Thus, these data provide evidence that therapeutic vaccines directed against WT1 will be able to elicit an immune response to WT1.

The majority of the antibodies detected were reactive with epitopes within the N-terminus while only a small subgroup of patients showed a weak antibody response to the C-terminus. This is consistent with observations in the animal model, where immunization with peptides derived from the N-terminus elicited antibody, helper T cell and CTL responses, whereas none of the peptides tested from the C-terminus elicited antibody or T cell responses (Gaiger et al., Blood 96:1334, 2000).

### Example 2

### Induction of Antibodies to WT1 in Mice Immunized with Cell Lines Expressing WT1

This Example illustrates the use of cells expressing WT1 to induce a WT1 specific antibody response in vivo.

Detection of existent antibodies to WT1 in patients with leukemia strongly implied that it is possible to immunize to WT1 protein to elicit immunity to WT1. To test whether immunity to WT1 can be generated by vaccination, mice were injected with TRAMP-C, a WT1 positive tumor cell line of B6 origin. Briefly, male B6 mice were immunized with 5×10⁶ TRAMP-C cells subcutaneously and boosted twice with 5×10⁶ cells at three week intervals. Three weeks after the final immunization, sera were obtained and single cell suspensions of spleens were prepared in RPMI 1640 medium (GIBCO) with 25 μM β-2-mercaptoethanol, 200 units of penicillin per ml, 10 mM L-glutamine, and 10% fetal bovine serum.

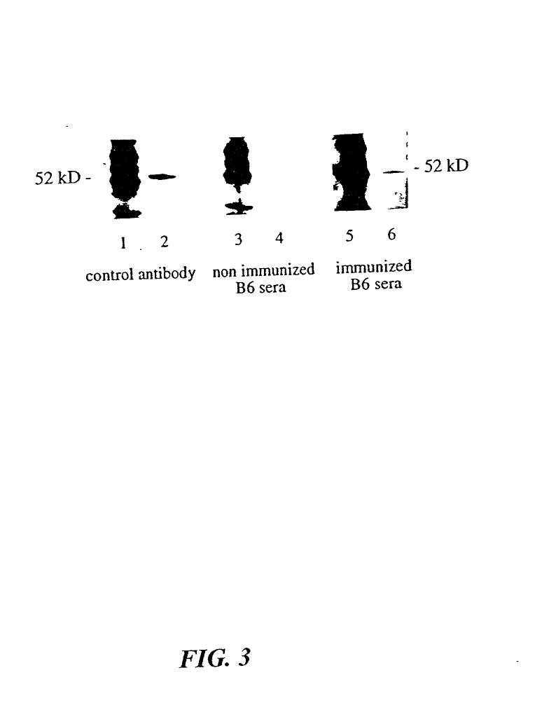

Following immunization to TRAMP-C, a WT1 specific antibody response in the immunized animals was detectable. A representative Western blot is shown in FIG. 3. These results show that immunization to WT1 protein can elicit an immune response to WT1 protein.

### EXAMPLE 3

### Induction of TH and Antibody Responses in Mice Immunized with WT1 Peptides

This Example illustrates the ability of immunization with WT1 peptides to elicit an immune response specific for WT1.

Peptides suitable for eliciting Ab and proliferative T cell responses were identified according to the Tsites program (Rothbard and Taylor, EMBO J. 7:93-100, 1988; Deavin et al., Mol. Immunol. 33:145-155, 1996), which searches for peptide motifs that have the potential to elicit Th responses. Peptides shown in Table I were synthesized and sequenced.

TABLE I

WT1 Peptides

| | | |
|---|---|---|
| Peptide | Sequence | Comments |

| | | | |
|---|---|---|---|
| Mouse: p6-22 | RDLNALLPAVSSLGGGG | (SEQ ID NO:13) | 1 mismatch relative to |
| | | | human WT1 sequence |
| Human: p6-22 | RDLNALLPAVPSLGGGG | (SEQ ID NO:1) | |
| Human/mouse: | PSQASSGQARMFPNAPYLPSCLE | (SEQ ID NOs:2 and 3) | |
| p117-139 | | | |
| Mouse: p244-262 | GATLKGMAAGSSSSVKWTE | (SEQ ID NO:14) | 1 mismatch relative to |
| | | | human WT1 sequence |
| Human: p244-262 | GATLKGVAAGSSSSVKWTE | (SEQ ID NO:4) | |
| Human/mouse: | RIHTHGVFRGIQDVR | (SEQ ID NOs:15 and 16) | |
| p287-301 | | | |
| Mouse: p299-313 | VRRVSGVAPTLVRS | (SEQ ID NO:17) | 1 mismatch relative to |
| | | | human WT1 sequence |
| Human mouse: | CQKKFARSDELVRHH | (SEQ ID NOs:19 and 20) | |
| p421-435 | | | |

For immunization, peptides were grouped as follows:

| | |
|---|---|
| Group A: | p6-22 human: 10.9 mg in 1 ml (10 μl = 100 μg) |
| | p117-139 human/mouse: 7.6 mg in 1 ml (14 μl = 100 μg) |
| | p244-262 human: 4.6. mg in 1 ml (22 μl = 100 μg) |
| Group B: | p287-301 human/mouse: 7.2 mg in 1 ml (14 μl = 100 μg) |
| | mouse p299-313: 6.6. mg in 1 ml (15 μl = 100 μg) |
| | p421-435 human/mouse: 3.3 mg in 1 ml (30 μl = 100 μg) |
| Control: | (FBL peptide 100 μg) + CFA/IFA |
| Control: | (CD45 peptide 100 μg) + CFA/IFA |

Group A contained peptides present within the amino terminus portion of WT1 (exon 1) and Group B contained peptides present within the carboxy terminus, which contains a four zinc finger region with sequence homology to other DNA-binding proteins. Within group B, p287-301 and p299-313 were derived from exon 7, zinc finger 1, and p421-435 was derived from exon 10, zinc finger IV.

B6 mice were immunized with a group of WT1 peptides or with a control peptide. Peptides were dissolved in 1 ml sterile water for injection, and B6 mice were immunized 3 times at time intervals of three weeks. Adjuvants used were CFA/IFA, GM-CSF, and Montinide. The presence of antibodies specific for WT1 was then determined as described in Examples 1 and 2, and proliferative T cell responses were evaluated using a standard thymidine incorporation assay, in which cells were cultured in the presence of antigen and proliferation was evaluated by measuring incorporated radioactivity (Chen et al., Cancer Res. 54:1065-1070, 1994). In particular, lymphocytes were cultured in 96-well plates at 2×10⁵ cells per well with 4×10⁵ irradiated (3000 rads) syngeneic spleen cells and the designated peptide.

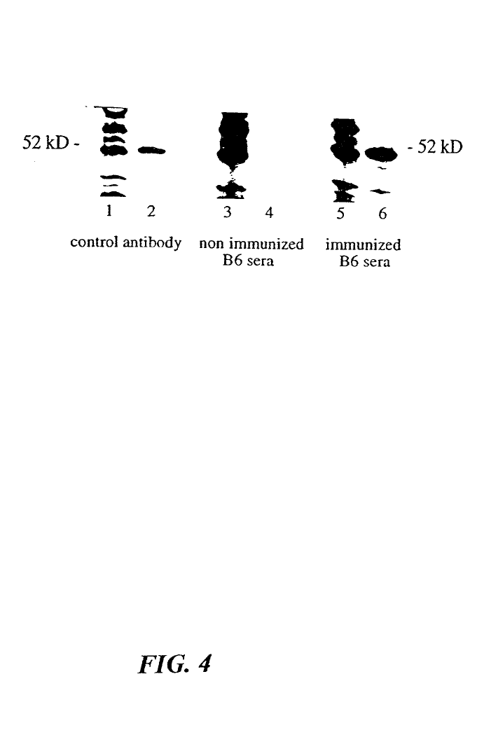

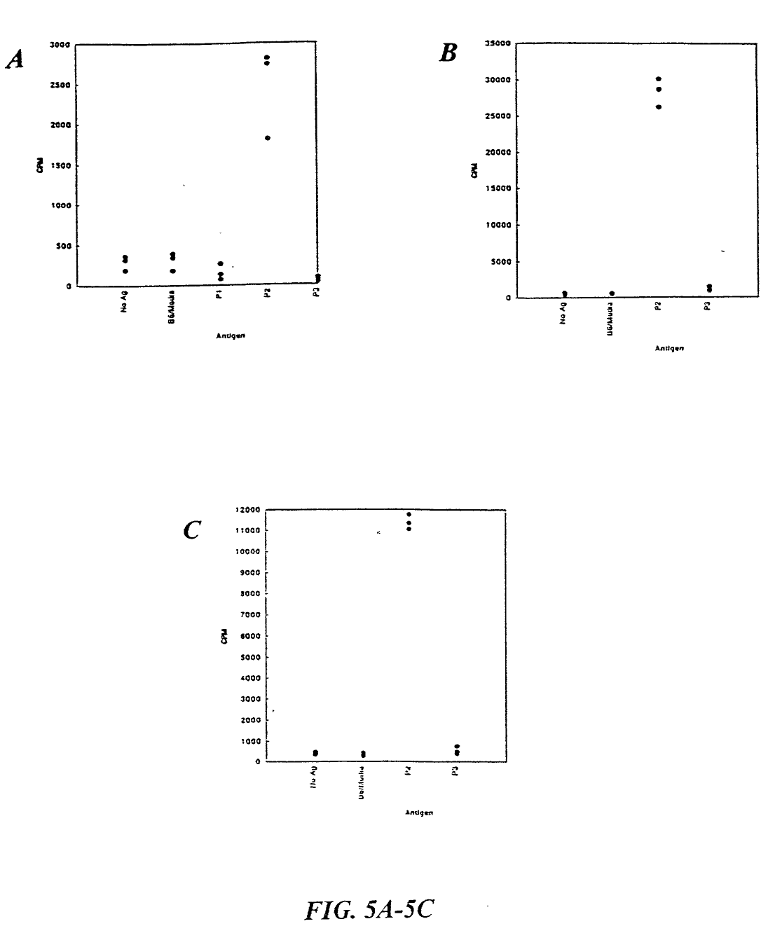

Immunization of mice with the group of peptides designated as Group A elicited an antibody response to WT1 (FIG. 4). No antibodies were detected following immunization to Vaccine B, which is consistent with a lack of helper T cell response from immunization with Vaccine B. P117-139 elicited proliferative T cell responses (FIGS. 5A-5C). The stimulation indices (SI) varied between 8 and 72. Other peptides (P6-22 and P299-313) also were shown to elicit proliferative T cell responses. Immunization with P6-22 resulted in a stimulation index (SI) of 2.3 and immunization with P299-313 resulted in a SI of 3.3. Positive controls included ConA stimulated T cells, as well as T cells stimulated with known antigens, such as CD45 and FBL, and allogeneic T cell lines (DeBruijn et al., Eur. J. Immunol. 21:2963-2970, 1991).

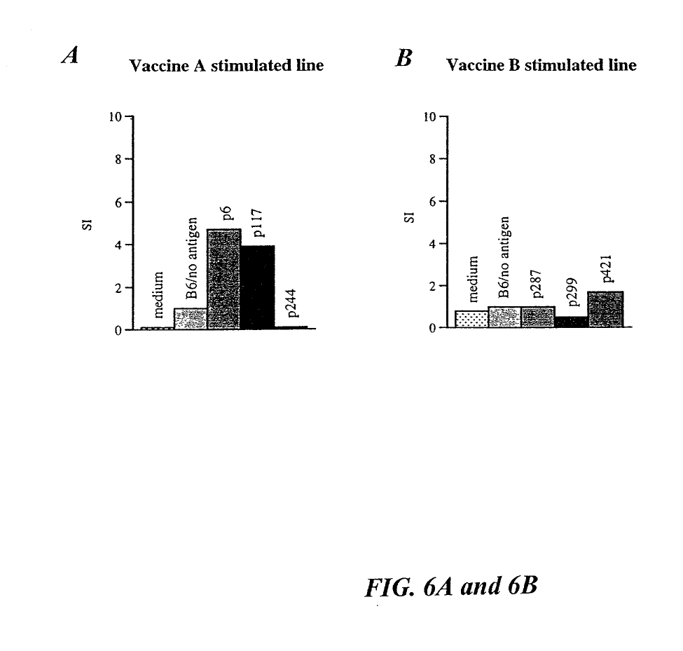

FIGS. 6A and 6B show the proliferative response observed for each of the three peptides within vaccine A (FIG. 6A) and vaccine B (FIG. 6B). Vaccine A elicited proliferative T cell responses to the immunizing peptides p6-22 and p117-139, with stimulation indices (SI) varying between 3 and 8 (bulk lines). No proliferative response to p244-262 was detected (FIG. 6A).

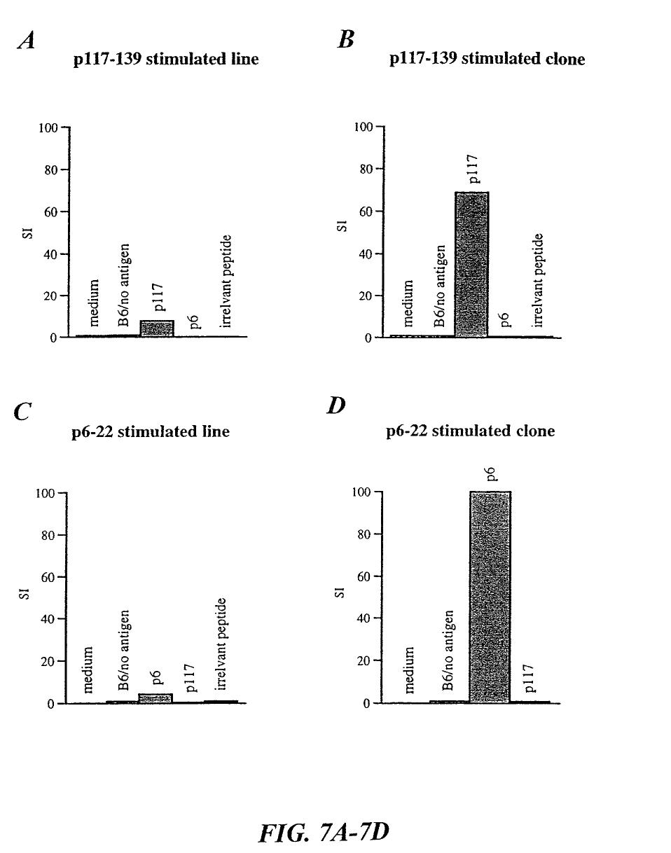

Subsequent in vitro stimulations were carried out as single peptide stimulations using only p6-22 and p117-139. Stimulation of the Vaccine A specific T cell line with p117-139 resulted in proliferation to p117-139 with no response to p6-22 (FIG. 7A). Clones derived from the line were specific for p117-139 (FIG. 7B). By contrast, stimulation of the Vaccine A specific T cell line with p6-22 resulted in proliferation to p6-22 with no response to p117-139 (FIG. 7C). Clones derived from the line were specific for p6-22 (FIG. 7D).

These results show that vaccination with WT1 peptides can elicit antibody responses to WT1 protein and proliferative T cell responses to the immunizing peptides.

### Example 4

### Induction of CTL Responses in Mice Immunized with WT1 Peptides

This Example illustrates the ability of WT1 peptides to elicit CTL immunity.

Peptides (9-mers) with motifs appropriate for binding to class I MHC were identified using a BIMAS HLA peptide binding prediction analysis (Parker et al., J. Immunol. 152:163, 1994). Peptides identified within such analyses are shown in Tables II-XLIV. In each of these tables, the score reflects the theoretical binding affinity (half-time of dissociation) of the peptide to the MHC molecule indicated.

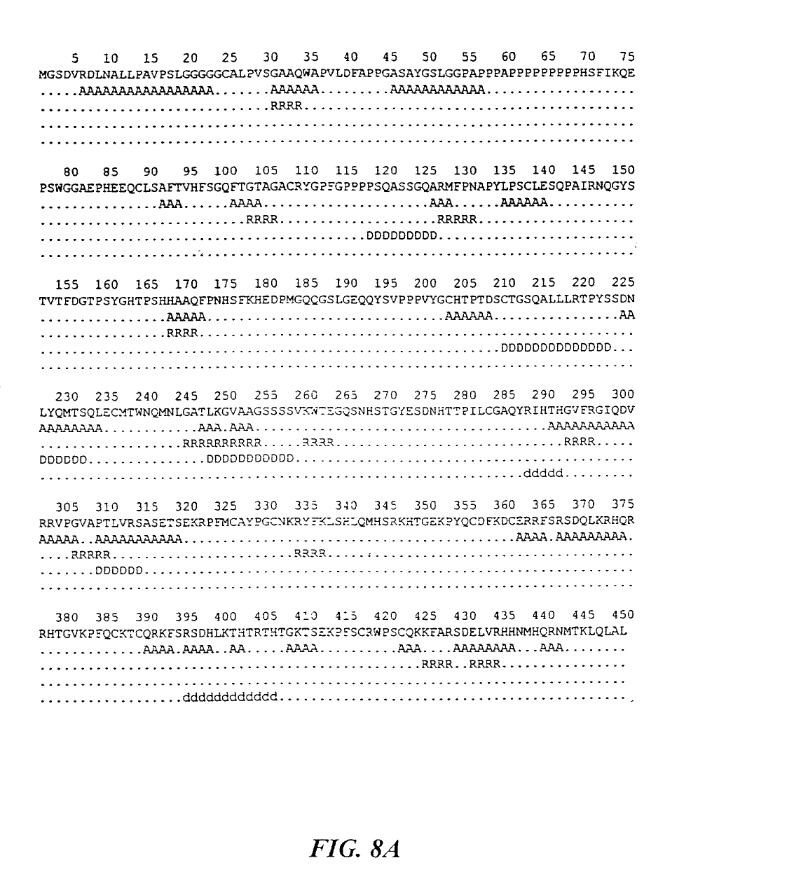

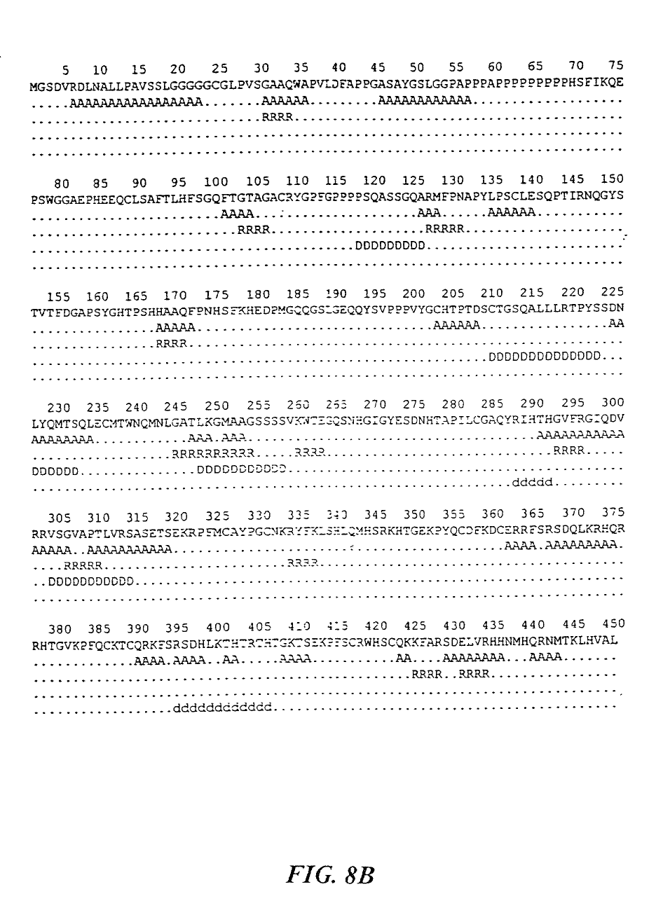

Peptides identified using the Tsites program (Rothbard and Taylor, EMBO J. 7:93-100, 1988; Deavin et al., Mol. Immunol. 33:145-155, 1996), which searches for peptide motifs that have the potential to elicit Th responses are further shown in FIGS. 8A and 8B, and Table XLV.

TABLE II

Results of BIMAS HLA Peptide Binding Prediction Analysis for
Binding of Human WT1 Peptides to Human HLA A1

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 137 | CLESQPAIR | (SEQ ID NO:47) | 18.000 |
| 2 | 80 | GAEPHEEQC | (SEQ ID NO:87) | 9.000 |
| 3 | 40 | FAPPGASAY | (SEQ ID NO:74) | 5.000 |
| 4 | 354 | QCDFKDCER | (SEQ ID NO:162) | 5.000 |
| 5 | 2 | GSDVRDLNA | (SEQ ID NO:101) | 3.750 |
| 6 | 152 | VTFDGTPSY | (SEQ ID NO:244) | 2.500 |
| 7 | 260 | WTEGQSNHS | (SEQ ID NO:247) | 2.250 |
| 8 | 409 | TSEKPFSCR | (SEQ ID NO:232) | 1.350 |
| 9 | 73 | KQEPSWGGA | (SEQ ID NO:125) | 1.350 |
| 10 | 386 | KTCQRKFSR | (SEQ ID NO:128) | 1.250 |
| 11 | 37 | VLDFAPPGA | (SEQ ID NO:241) | 1.000 |
| 12 | 325 | CAYPGCNKR | (SEQ ID NO:44) | 1.000 |
| 13 | 232 | QLECMTWNQ | (SEQ ID NO:167) | 0.900 |
| 14 | 272 | ESDNHTTPI | (SEQ ID NO:71) | 0.750 |
| 15 | 366 | RSDQLKRHQ | (SEQ ID NO:193) | 0.750 |
| 16 | 222 | SSDNLYQMT | (SEQ ID NO:217) | 0.750 |
| 17 | 427 | RSDELVRHH | (SEQ ID NO:191) | 0.750 |
| 18 | 394 | RSDHLKTHT | (SEQ ID NO:192) | 0.750 |
| 19 | 317 | TSEKRPFMC | (SEQ ID NO:233) | 0.675 |
| 20 | 213 | QALLLRTPY | (SEQ ID NO:160) | 0.500 |

TABLE III

Results of BIMAS HLA Peptide Binding Prediction Analysis for
Binding of Human WT1 Peptides to Human HLA A 0201

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 126 | RMFPNAPYL | (SEQ ID NO:185) | 313.968 |
| 2 | 187 | SLGEQQYSV | (SEQ ID NO:214) | 285.163 |
| 3 | 10 | ALLPAVPSL | (SEQ ID NO:34) | 181.794 |
| 4 | 242 | NLGATLKGV | (SEQ ID NO:146) | 159.970 |
| 5 | 225 | NLYQMTSQL | (SEQ ID NO:147) | 68.360 |
| 6 | 292 | GVFRGIQDV | (SEQ ID NO:103) | 51.790 |
| 7 | 191 | QQYSVPPPV | (SEQ ID NO:171) | 22.566 |
| 8 | 280 | ILCGAQYRI | (SEQ ID NO:116) | 17.736 |
| 9 | 235 | CMTWNQMNL | (SEQ ID NO:49) | 15.428 |
| 10 | 441 | NMTKLQLAL | (SEQ ID NO:149) | 15.428 |
| 11 | 7 | DLNALLPAV | (SEQ ID NO:58) | 11.998 |
| 12 | 227 | YQMTSQLEC | (SEQ ID NO:251) | 8.573 |
| 13 | 239 | NQMNLGATL | (SEQ ID NO:151) | 8.014 |
| 14 | 309 | TLVRSASET | (SEQ ID NO:226) | 7.452 |
| 15 | 408 | KTSEKPFSC | (SEQ ID NO:129) | 5.743 |
| 16 | 340 | LQMHSRKHT | (SEQ ID NO:139) | 4.752 |
| 17 | 228 | QMTSQLECM | (SEQ ID NO:169) | 4.044 |
| 18 | 93 | TVHFSGQFT | (SEQ ID NO:235) | 3.586 |
| 19 | 37 | VLDFAPPGA | (SEQ ID NO:241) | 3.378 |
| 20 | 86 | EQCLSAFTV | (SEQ ID NO:69) | 3.068 |

TABLE IV

Results of BIMAS HLA Peptide Binding Prediction Analysis for
Binding of Human WT1 Peptides to Human HLA A 0205

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 10 | ALLPAVPSL | (SEQ ID NO:34) | 42.000 |
| 2 | 292 | GVFRGIQDV | (SEQ ID NO:103) | 24.000 |
| 3 | 126 | RMFPNAPYL | (SEQ ID NO:185) | 21.000 |
| 4 | 225 | NLYQMTSQL | (SEQ ID NO:147) | 21.000 |
| 5 | 239 | NQMNLGATL | (SEQ ID NO:151) | 16.800 |
| 6 | 302 | RVPGVAPTL | (SEQ ID NO:195) | 14.000 |
| 7 | 441 | NMTKLQLAL | (SEQ ID NO:149) | 7.000 |
| 8 | 235 | CMTWNQMNL | (SEQ ID NO:49) | 7.000 |
| 9 | 187 | SLGEQQYSV | (SEQ ID NO:214) | 6.000 |
| 10 | 191 | QQYSVPPPV | (SEQ ID NO:171) | 4.800 |
| 11 | 340 | LQMHSRKHT | (SEQ ID NO:139) | 4.080 |
| 12 | 242 | NLGATLKGV | (SEQ ID NO:146) | 4.000 |
| 13 | 227 | YQMTSQLEC | (SEQ ID NO:251) | 3.600 |
| 14 | 194 | SVPPPVYGC | (SEQ ID NO:218) | 2.000 |
| 15 | 93 | TVHFSGQFT | (SEQ ID NO:235) | 2.000 |
| 16 | 280 | ILCGAQYRI | (SEQ ID NO:116) | 1.700 |
| 17 | 98 | GQFTGTAGA | (SEQ ID NO:99) | 1.200 |
| 18 | 309 | TLVRSASET | (SEQ ID NO:226) | 1.000 |
| 19 | 81 | AEPHEEQCL | (SEQ ID NO:30) | 0.980 |
| 20 | 73 | KQEPSWGGA | (SEQ ID NO:125) | 0.960 |

TABLE V

Results of BIMAS HLA Peptide Binding Prediction Analysis for
Binding of Human WT1 Peptides to Human HLA A24

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 302 | RVPGVAPTL | (SEQ ID NO:195) | 16.800 |
| 2 | 218 | RTPYSSDNL | (SEQ ID NO:194) | 12.000 |
| 3 | 356 | DFKDCERRF | (SEQ ID NO:55) | 12.000 |
| 4 | 126 | RMFPNAPYL | (SEQ ID NO:185) | 9.600 |
| 5 | 326 | AYPGCNKRY | (SEQ ID NO:42) | 7.500 |
| 6 | 270 | GYESDNHT | (SEQ ID NO:106)T | 7.500 |
| 7 | 239 | NQMNLGATL | (SEQ ID NO:151) | 7.200 |
| 8 | 10 | ALLPAVPSL | (SEQ ID NO:34) | 7.200 |
| 9 | 130 | NAPYLPSCL | (SEQ ID NO:144) | 7.200 |
| 10 | 329 | GCNKRYFKL | (SEQ ID NO:90) | 6.600 |
| 11 | 417 | RWPSCQKKF | (SEQ ID NO:196) | 6.600 |
| 12 | 47 | AYGSLGGPA | (SEQ ID NO:41) | 6.000 |
| 13 | 180 | DPMGQQGSL | (SEQ ID NO:59) | 6.000 |
| 14 | 4 | DVRDLNALL | (SEQ ID NO:62) | 5.760 |
| 15 | 285 | QYRIHTHGV | (SEQ ID NO:175) | 5.000 |
| 16 | 192 | QYSVPPPVY | (SEQ ID NO:176) | 5.000 |
| 17 | 207 | DSCTGSQAL | (SEQ ID NO:61) | 4.800 |
| 18 | 441 | NMTKLQLAL | (SEQ ID NO:149) | 4.800 |
| 19 | 225 | NLYQMTSQL | (SEQ ID NO:147) | 4.000 |
| 20 | 235 | CMTWNQMNL | (SEQ ID NO:49) | 4.000 |

TABLE VI

Results of BIMAS HLA Peptide Binding Prediction Analysis for
Binding of Human WT1 Peptides to Human HLA A3

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 436 | NMHQRNMTK | (SEQ ID NO:148) | 40.000 |
| 2 | 240 | QMNLGATLK | (SEQ ID NO:168) | 20.000 |
| 3 | 88 | CLSAFTVHF | (SEQ ID NO:48) | 6.000 |
| 4 | 126 | RMFPNAPYL | (SEQ ID NO:185) | 4.500 |
| 5 | 169 | AQFPNHSFK | (SEQ ID NO:36) | 4.500 |
| 6 | 10 | ALLPAVPSL | (SEQ ID NO:34) | 4.050 |
| 7 | 137 | CLESQPAIR | (SEQ ID NO:47) | 4.000 |
| 8 | 225 | NLYQMTSQL | (SEQ ID NO:147) | 3.000 |
| 9 | 32 | AQWAPVLDF | (SEQ ID NO:37) | 2.700 |
| 10 | 280 | ILCGAQYRI | (SEQ ID NO:116) | 2.700 |
| 11 | 386 | KTCQRKFSR | (SEQ ID NO:128) | 1.800 |
| 12 | 235 | CMTWNQMNL | (SEQ ID NO:49) | 1.200 |
| 13 | 441 | NMTKLQLAL | (SEQ ID NO:149) | 1.200 |
| 14 | 152 | VTFDGTPSY | (SEQ ID NO:244) | 1.000 |
| 15 | 187 | SLGEQQYSV | (SEQ ID NO:214) | 0.900 |
| 16 | 383 | FQCKTCQRK | (SEQ ID NO:80) | 0.600 |
| 17 | 292 | GVFRGIQDV | (SEQ ID NO:103) | 0.450 |
| 18 | 194 | SVPPPVYGC | (SEQ ID NO:218) | 0.405 |
| 19 | 287 | RIHTHGVFR | (SEQ ID NO:182) | 0.400 |
| 20 | 263 | GQSNHSTGY | (SEQ ID NO:100) | 0.360 |

TABLE VII

Results of BIMAS HLA Peptide Binding Prediction Analysis for
Binding of Human WT1 Peptides to Human HLA A68.1

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 100 | FTGTAGACR | (SEQ ID NO:84) | 100.000 |
| 2 | 386 | KTCQRKFSR | (SEQ ID NO:128) | 50.000 |
| 3 | 368 | DQLKRHQRR | (SEQ ID NO:60) | 30.000 |
| 4 | 312 | RSASETSEK | (SEQ ID NO:190) | 18.000 |
| 5 | 337 | LSHLQMHSR | (SEQ ID NO:141) | 15.000 |
| 6 | 364 | FSRSDQLKR | (SEQ ID NO:83) | 15.000 |
| 7 | 409 | TSEKPFSCR | (SEQ ID NO:232) | 15.000 |
| 8 | 299 | DVRRVPGVA | (SEQ ID NO:63) | 12.000 |
| 9 | 4 | DVRDLNALL | (SEQ ID NO:62) | 12.000 |
| 10 | 118 | SQASSGQAR | (SEQ ID NO:216) | 10.000 |
| 11 | 343 | HSRKHTGEK | (SEQ ID NO:111) | 9.000 |
| 12 | 169 | AQFPNHSFK | (SEQ ID NO:36) | 9.000 |
| 13 | 292 | GVFRGIQDV | (SEQ ID NO:103) | 8.000 |
| 14 | 325 | CAYPGCNKR | (SEQ ID NO:44) | 7.500 |
| 15 | 425 | FARSDELVR | (SEQ ID NO:75) | 7.500 |
| 16 | 354 | QCDFKDCER | (SEQ ID NO:162) | 7.500 |
| 17 | 324 | MCAYPGCNK | (SEQ ID NO:142) | 6.000 |
| 18 | 251 | AAGSSSSVK | (SEQ ID NO:28) | 6.000 |
| 19 | 379 | GVKPFQCKT | (SEQ ID NO:104) | 6.000 |
| 20 | 137 | CLESQPAIR | (SEQ ID NO:47) | 5.000 |

TABLE VIII

Results of BIMAS HLA Peptide Binding Prediction Analysis for
Binding of Human WT1 Peptides to Human HLA A 1101

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 386 | KTCQRKFSR | (SEQ ID NO:128) | 1.800 |
| 2 | 169 | AQFPNHSFK | (SEQ ID NO:36) | 1.200 |
| 3 | 436 | NMHQRNMTK | (SEQ ID NO:148) | 0.800 |
| 4 | 391 | KFSRSDHLK | (SEQ ID NO:120) | 0.600 |
| 5 | 373 | HQRRHTGVK | (SEQ ID NO:109) | 0.600 |
| 6 | 383 | FQCKTCQRK | (SEQ ID NO:80) | 0.600 |
| 7 | 363 | RFSRSDQLK | (SEQ ID NO:178) | 0.600 |
| 8 | 240 | QMNLGATLK | (SEQ ID NO:168) | 0.400 |
| 9 | 287 | RIHTHGVFR | (SEQ ID NO:182) | 0.240 |
| 10 | 100 | FTGTAGACR | (SEQ ID NO:84) | 0.200 |
| 11 | 324 | MCAYPGCNK | (SEQ ID NO:142) | 0.200 |
| 12 | 251 | AAGSSSSVK | (SEQ ID NO:28) | 0.200 |
| 13 | 415 | SCRWPSCQK | (SEQ ID NO:201) | 0.200 |
| 14 | 118 | SQASSGQAR | (SEQ ID NO:216) | 0.120 |
| 15 | 292 | GVFRGIQDV | (SEQ ID NO:103) | 0.120 |
| 16 | 137 | CLESQPAIR | (SEQ ID NO:47) | 0.080 |
| 17 | 425 | FARSDELVR | (SEQ ID NO:75) | 0.080 |
| 18 | 325 | CAYPGCNKR | (SEQ ID NO:44) | 0.080 |
| 19 | 312 | RSASETSEK | (SEQ ID NO:190) | 0.060 |
| 20 | 65 | PPPPHSFI | (SEQ ID NO:156)K | 0.060 |

TABLE IX

Results of BIMAS HLA Peptide Binding Prediction Analysis for
Binding of Human WT1 Peptides to Human HLA A 3101

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 386 | KTCQRKESR | (SEQ ID NO:128) | 9.000 |
| 2 | 287 | RIHTHGVFR | (SEQ ID NO:182) | 6.000 |
| 3 | 137 | CLESQPAIR | (SEQ ID NO:47) | 2.000 |
| 4 | 118 | SQASSGQAR | (SEQ ID NO:216) | 2.000 |
| 5 | 368 | DQLKRHQRR | (SEQ ID NO:60) | 1.200 |
| 6 | 100 | FTGTAGACR | (SEQ ID NO:84) | 1.000 |
| 7 | 293 | VFRGIQDVR | (SEQ ID NO:238) | 0.600 |
| 8 | 325 | CAYPGCNKR | (SEQ ID NO:44) | 0.600 |
| 9 | 169 | AQFPNHSFK | (SEQ ID NO:36) | 0.600 |
| 10 | 279 | PILCGAQYR | (SEQ ID NO:155) | 0.400 |
| 11 | 436 | NMHQRNMTK | (SEQ ID NO:148) | 0.400 |
| 12 | 425 | FARSDELVR | (SEQ ID NO:75) | 0.400 |
| 13 | 32 | AQWAPVLDF | (SEQ ID NO:37) | 0.240 |
| 14 | 240 | QMNLGATLK | (SEQ ID NO:168) | 0.200 |
| 15 | 354 | QCDFKDCER | (SEQ ID NO:162) | 0.200 |
| 16 | 373 | HQRRHTGVK | (SEQ ID NO:109) | 0.200 |
| 17 | 383 | FQCKTCQRK | (SEQ ID NO:80) | 0.200 |
| 18 | 313 | SASETSEKR | (SEQ ID NO:197) | 0.200 |
| 19 | 358 | KDCERRFSR | (SEQ ID NO:118) | 0.180 |
| 20 | 391 | KFSRSDHLK | (SEQ ID NO:120) | 0.180 |

TABLE X

Results of BIMAS HLA Peptide Binding Prediction Analysis for
Binding of Human WT1 Peptides to Human HLA A 3302

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 337 | LSHLQMHSR | (SEQ ID NO:141) | 15.000 |
| 2 | 409 | TSEKPFSCR | (SEQ ID NO:232) | 15.000 |
| 3 | 364 | FSRSDQLKR | (SEQ ID NO:83) | 15.000 |
| 4 | 137 | CLESQPAIR | (SEQ ID NO:47) | 9.000 |
| 5 | 368 | DQLKRHQRR | (SEQ ID NO:60) | 9.000 |
| 6 | 287 | RIHTHGVFR | (SEQ ID NO:182) | 4.500 |
| 7 | 210 | TGSQALLLR | (SEQ ID NO:223) | 3.000 |
| 8 | 425 | FARSDELVR | (SEQ ID NO:75) | 3.000 |
| 9 | 313 | SASETSEKR | (SEQ ID NO:197) | 3.000 |
| 10 | 293 | VFRGIQDVR | (SEQ ID NO:238) | 3.000 |
| 11 | 354 | QCDFKDCER | (SEQ ID NO:162) | 3.000 |
| 12 | 100 | FTGTAGACR | (SEQ ID NO:84) | 3.000 |
| 13 | 118 | SQASSGQAR | (SEQ ID NO:216) | 3.000 |
| 14 | 325 | CAYPGCNKR | (SEQ ID NO:44) | 3.000 |
| 15 | 207 | DSCTGSQAL | (SEQ ID NO:61) | 1.500 |
| 16 | 139 | ESQPAIRNQ | (SEQ ID NO:72) | 1.500 |
| 17 | 299 | DVRRVPGVA | (SEQ ID NO:63) | 1.500 |
| 18 | 419 | PSCQKKFAR | (SEQ ID NO:159) | 1.500 |
| 19 | 272 | ESDNHTTPI | (SEQ ID NO:71) | 1.500 |
| 20 | 4 | DVRDLNALL | (SEQ ID NO:62) | 1.500 |

TABLE XI

| Results of BIMAS HLA Peptide Binding Prediction Analysis for | | | | |
|---|---|---|---|---|
| Binding of Human WT1 Peptides to Human HLA B14 | | | | |
| | | | | Score (Estimate of Half Time |
| | | | | of Disassociation of a |
| | Start | | | Molecule Containing This |
| Rank | Position | Subsequence Residue Listing | | Subsequence) |
| 1 | 362 | RRFSRSDQL | (SEQ ID NO:187) | 1000.000 |
| 2 | 332 | KRYFKLSHL | (SEQ ID NO:127) | 300.000 |
| 3 | 423 | KKFARSDEL | (SEQ ID NO:122) | 150.000 |
| 4 | 390 | RKFSRSDHL | (SEQ ID NO:183) | 150.000 |
| 5 | 439 | QRNMTKLQL | (SEQ ID NO:173) | 20.000 |
| 6 | 329 | GCNKLRYFKL | (SEQ ID NO:90) | 10.000 |
| 7 | 10 | ALLPAVPSL | (SEQ ID NO:34) | 10.000 |
| 8 | 180 | DPMGQQGSL | (SEQ ID NO:59) | 9.000 |
| 9 | 301 | RRVPGVAPT | (SEQ ID NO:189) | 6.000 |
| 10 | 126 | RMFPNAPYL | (SEQ ID NO:185) | 5.000 |
| 11 | 371 | KRHQRRHTG | (SEQ ID NO:126) | 5.000 |
| 12 | 225 | NLYQMTSQL | (SEQ ID NO:147) | 5.000 |
| 13 | 144 | IRNQGYSTV | (SEQ ID NO:117) | 4.000 |
| 14 | 429 | DELVRHHNM | (SEQ ID NO:53) | 3.000 |
| 15 | 437 | MHQRNMTKL | (SEQ ID NO:143) | 3.000 |
| 16 | 125 | ARMFPNAPY | (SEQ ID NO:38) | 3.000 |
| 17 | 239 | NQMNLGATL | (SEQ ID NO:151) | 3.000 |
| 18 | 286 | YRIHTHGVF | (SEQ ID NO:252) | 3.000 |
| 19 | 174 | HSFKHEDPM | (SEQ ID NO:110) | 3.000 |
| 20 | 372 | RHQRRHTGV | (SEQ ID NO:181) | 3.000 |

TABLE XII
Results of BIMAS HLA Peptide Binding Prediction Analysis for
Binding of Human WT1 Peptides to Human HLA B40

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 81 | AEPHEEQCL | (SEQ ID NO:30) | 40.000 |
| 2 | 429 | DELVRHHNM | (SEQ ID NO:53) | 24.000 |
| 3 | 410 | SEKPFSCRW | (SEQ ID NO:207) | 20.000 |
| 4 | 318 | SEKRPFMCA | (SEQ ID NO:208) | 15.000 |
| 5 | 233 | LECMTWNQM | (SEQ ID NO:131) | 12.000 |
| 6 | 3 | SDVRDLNAL | (SEQ ID NO:206) | 10.000 |
| 7 | 349 | GEKPYQCDF | (SEQ ID NO:91) | 8.000 |
| 8 | 6 | RDLNALLPA | (SEQ ID NO:177) | 5.000 |
| 9 | 85 | EEQCLSAFT | (SEQ ID NO:65) | 4.000 |
| 10 | 315 | SETSEKRPF | (SEQ ID NO:209) | 4.000 |
| 11 | 261 | TEGQSNHST | (SEQ ID NO:221) | 4.000 |
| 12 | 23 | GCALPVSGA | (SEQ ID NO:89) | 3.000 |
| 13 | 38 | LDFAPPGAS | (SEQ ID NO:130) | 3.000 |
| 14 | 273 | SDNHTTPIL | (SEQ ID NO:204) | 2.500 |
| 15 | 206 | TDSCTGSQA | (SEQ ID NO:220) | 2.500 |
| 16 | 24 | CALPVSGAA | (SEQ ID NO:43) | 2.000 |
| 17 | 98 | GQFTGTAGA | (SEQ ID NO:99) | 2.000 |
| 18 | 30 | GAAQWAPVL | (SEQ ID NO:86) | 2.000 |
| 19 | 84 | HEEQCLSAF | (SEQ ID NO:107) | 2.000 |
| 20 | 26 | LPVSGAAQW | (SEQ ID NO:138) | 2.000 |

TABLE XIII
Results of BIMAS HLA Peptide Binding Prediction Analysis for
Binding of Human WT1 Peptides to Human HLA B60

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 81 | AEPHEEQCL | (SEQ ID NO:30) | 160.000 |
| 2 | 3 | SDVRDLNAL | (SEQ ID NO:206) | 40.000 |
| 3 | 429 | DELVRHHNM | (SEQ ID NO:53) | 40.000 |
| 4 | 233 | LECMTWNQM | (SEQ ID NO:131) | 22.000 |
| 5 | 273 | SDNHTTPIL | (SEQ ID NO:204) | 20.000 |
| 6 | 209 | CTGSQALLL | (SEQ ID NO:52) | 8.000 |
| 7 | 30 | GAAQWAPVL | (SEQ ID NO:86) | 8.000 |
| 8 | 318 | SEKRPFMCA | (SEQ ID NO:208) | 8.000 |
| 9 | 180 | DPMGQQGSL | (SEQ ID NO:59) | 8.000 |
| 10 | 138 | LESQPAIRN | (SEQ ID NO:132) | 5.280 |
| 11 | 239 | NQMNLGATL | (SEQ ID NO:151) | 4.400 |
| 12 | 329 | GCNKRYFKL | (SEQ ID NO:90) | 4.400 |
| 13 | 130 | NAPYLPSCL | (SEQ ID NO:144) | 4.400 |
| 14 | 85 | EEQCLSAFT | (SEQ ID NO:65) | 4.400 |
| 15 | 208 | SCTGSQALL | (SEQ ID NO:202) | 4.000 |
| 16 | 207 | DSCTGSQAL | (SEQ ID NO:61) | 4.000 |
| 17 | 218 | RTPYSSDNL | (SEQ ID NO:194) | 4.000 |
| 18 | 261 | TEGQSNHST | (SEQ ID NO:221) | 4.000 |
| 19 | 18 | LGGGGGCAL | (SEQ ID NO:134) | 4.000 |
| 20 | 221 | YSSDNLYQM | (SEQ ID NO:253) | 2.200 |

TABLE XIV
Results of BIMAS HLA Peptide Binding Prediction Analysis for
Binding of Human WT1 Peptides to Human HLA B61

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 318 | SEKRPFMCA | (SEQ ID NO:208) | 20.000 |
| 2 | 429 | DELVRHHNM | (SEQ ID NO:53) | 16.000 |
| 3 | 298 | QDVRRVPGV | (SEQ ID NO:164) | 10.000 |
| 4 | 81 | AEPHEEQCL | (SEQ ID NO:30) | 8.000 |
| 5 | 233 | LECMTWNQM | (SEQ ID NO:131) | 8.000 |
| 6 | 6 | RDLNALLPA | (SEQ ID NO:177) | 5.500 |
| 7 | 85 | EEQCLSAFT | (SEQ ID NO:65) | 4.000 |
| 8 | 261 | TEGQSNHST | (SEQ ID NO:221) | 4.000 |
| 9 | 206 | TDSCTGSQA | (SEQ ID NO:220) | 2.500 |
| 10 | 295 | RGIQDVRRV | (SEQ ID NO:179) | 2.200 |
| 11 | 3 | SDVRDLNAL | (SEQ ID NO:206) | 2.000 |
| 12 | 250 | VAAGSSSSV | (SEQ ID NO:236) | 2.000 |
| 13 | 29 | SGAAQWAPV | (SEQ ID NO:211) | 2.000 |
| 14 | 315 | SETSEKRPF | (SEQ ID NO:209) | 1.600 |
| 15 | 138 | LESQPAIRN | (SEQ ID NO:132) | 1.200 |
| 16 | 244 | GATLKGVAA | (SEQ ID NO:88) | 1.100 |
| 17 | 20 | GGGGCALPV | (SEQ ID NO:92) | 1.100 |
| 18 | 440 | RNMTKLQLA | (SEQ ID NO:186) | 1.100 |
| 19 | 23 | GCALPVSGA | (SEQ ID NO:89) | 1.100 |
| 20 | 191 | QQYSVPPPV | (SEQ ID NO:171) | 1.000 |

TABLE XV
Results of BIMAS HLA Peptide Binding Prediction Analysis for
Binding of Human WT1 Peptides to Human HLA B62

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 146 | NQGYSTVTF | (SEQ ID NO:150) | 211.200 |
| 2 | 32 | AQWAPVLDF | (SEQ ID NO:37) | 96.000 |
| 3 | 263 | GQSNHSTGY | (SEQ ID NO:100) | 96.000 |
| 4 | 88 | CLSAFTVHF | (SEQ ID NO:48) | 96.000 |
| 5 | 17 | SLGGGGGCA | (SEQ ID NO:215) | 9.600 |
| 6 | 239 | NQMNLGATL | (SEQ ID NO:151) | 8.800 |
| 7 | 191 | QQYSVPPPV | (SEQ ID NO:171) | 8.000 |
| 8 | 98 | GQFTGTAGA | (SEQ ID NO:99) | 8.000 |
| 9 | 384 | QCKTCQRKF | (SEQ ID NO:163) | 6.000 |
| 10 | 40 | FAPPGASAY | (SEQ ID NO:74) | 4.800 |
| 11 | 227 | YQMTSQLEC | (SEQ ID NO:251) | 4.800 |
| 12 | 187 | SLGEQQYSV | (SEQ ID NO:214) | 4.400 |
| 13 | 86 | EQCLSAFTV | (SEQ ID NO:69) | 4.400 |
| 14 | 152 | VTFDGTPSY | (SEQ ID NO:244) | 4.400 |
| 15 | 101 | TGTAGACRY | (SEQ ID NO:224) | 4.000 |
| 16 | 242 | NLGATLKGV | (SEQ ID NO:146) | 4.000 |
| 17 | 92 | FTVHFSGQF | (SEQ ID NO:85) | 4.000 |
| 18 | 7 | DLNALLPAV | (SEQ ID NO:58) | 4.000 |
| 19 | 123 | GQARMFPNA | (SEQ ID NO:98) | 4.000 |
| 20 | 280 | ILCGAQYRI | (SEQ ID NO:116) | 3.120 |

TABLE XVI
Results of BIMAS HLA Peptide Binding Prediction Analysis for
Binding of Human WT1 Peptides to Human HLA B7

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 180 | DPMGQQGSL | (SEQ ID NO:59) | 240.000 |
| 2 | 4 | DVRDLNALL | (SEQ ID NO:62) | 200.000 |
| 3 | 302 | RVPGVAPTL | (SEQ ID NO:195) | 20.000 |
| 4 | 30 | GAAQWAPVL | (SEQ ID NO:86) | 12.000 |
| 5 | 239 | NQMNLGATL | (SEQ ID NO:151) | 12.000 |
| 6 | 130 | NAPYLPSCL | (SEQ ID NO:144) | 12.000 |
| 7 | 10 | ALLPAVPSL | (SEQ ID NO:34) | 12.000 |
| 8 | 299 | DVRRVPGVA | (SEQ ID NO:63) | 5.000 |
| 9 | 208 | SCTGSQALL | (SEQ ID NO:202) | 4.000 |
| 10 | 303 | VPGVAPTLV | (SEQ ID NO:242) | 4.000 |
| 11 | 18 | LGGGGGCAL | (SEQ ID NO:134) | 4.000 |
| 12 | 218 | RTPYSSDNL | (SEQ ID NO:194) | 4.000 |
| 13 | 207 | DSCTGSQAL | (SEQ ID NO:61) | 4.000 |
| 14 | 209 | CTGSQALLL | (SEQ ID NO:52) | 4.000 |
| 15 | 329 | GCNKRYFKL | (SEQ ID NO:90) | 4.000 |
| 16 | 235 | CMTWNQMNL | (SEQ ID NO:49) | 4.000 |
| 17 | 441 | NMTKLQLAL | (SEQ ID NO:149) | 4.000 |
| 18 | 126 | RMFPNAPYL | (SEQ ID NO:185) | 4.000 |
| 19 | 225 | NLYQMTSQL | (SEQ ID NO:147) | 4.000 |
| 20 | 143 | AIRNQGYST | (SEQ ID NO:33) | 3.000 |

TABLE XVII
Results of BIMAS HLA Peptide Binding Prediction Analysis for
Binding of Human WT1 Peptides to Human HLA B8

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 329 | GCNKRYFKL | (SEQ ID NO:90) | 16.000 |
| 2 | 4 | DVRDLNALL | (SEQ ID NO:62) | 12.000 |
| 3 | 316 | ETSEKRPFM | (SEQ ID NO:73) | 3.000 |
| 4 | 180 | DPMGQQGSL | (SEQ ID NO:59) | 1.600 |
| 5 | 208 | SCTGSQALL | (SEQ ID NO:202) | 0.800 |
| 6 | 130 | NAPYLPSCL | (SEQ ID NO:144) | 0.800 |
| 7 | 244 | GATLKGVAA | (SEQ ID NO:88) | 0.800 |
| 8 | 30 | GAAQWAPVL | (SEQ ID NO:86) | 0.800 |
| 9 | 299 | DVRRVPGVA | (SEQ ID NO:63) | 0.400 |
| 10 | 420 | SCQKKFARS | (SEQ ID NO:200) | 0.400 |
| 11 | 387 | TCQRKFSRS | (SEQ ID NO:219) | 0.400 |
| 12 | 225 | NLYQMTSQL | (SEQ ID NO:147) | 0.400 |
| 13 | 141 | QPAIRNQGY | (SEQ ID NO:170) | 0.400 |
| 14 | 10 | ALLPAVPSL | (SEQ ID NO:34) | 0.400 |
| 15 | 207 | DSCTGSQAL | (SEQ ID NO:61) | 0.400 |
| 16 | 384 | QCKTCQRKF | (SEQ ID NO:163) | 0.400 |
| 17 | 136 | SCLESQPAI | (SEQ ID NO:198) | 0.300 |
| 18 | 347 | HTGEKPYQC | (SEQ ID NO:112) | 0.300 |
| 19 | 401 | HTRTHTGKT | (SEQ ID NO:114) | 0.200 |
| 20 | 332 | KRYFKLSHL | (SEQ ID NO:127) | 0.200 |

TABLE XVIII
Results of BIMAS HLA Peptide Binding Prediction Analysis for
Binding of Human WTl Peptides to Human HLA B2702

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 332 | KRYFKLSHL | (SEQ ID NO:127) | 900.000 |
| 2 | 362 | RRFSRSDQL | (SEQ ID NO:187) | 900.000 |
| 3 | 286 | YRIHTHGVF | (SEQ ID NO:252) | 200.000 |
| 4 | 125 | ARMFPNAPY | (SEQ ID NO:38) | 200.000 |
| 5 | 375 | RRHTGVKPF | (SEQ ID NO:188) | 180.000 |
| 6 | 32 | AQWAPVLDF | (SEQ ID NO:37) | 100.000 |
| 7 | 301 | RRVPGVAPT | (SEQ ID NO:189) | 60.000 |
| 8 | 439 | QRNMTKLQL | (SEQ ID NO:173) | 60.000 |
| 9 | 126 | RMFPNAPYL | (SEQ ID NO:185) | 22.500 |
| 10 | 426 | ARSDELVRH | (SEQ ID NO:39) | 20.000 |
| 11 | 146 | NQGYSTVTF | (SEQ ID NO:150) | 20.000 |
| 12 | 144 | IRNQGYSTV | (SEQ ID NO:117) | 20.000 |
| 13 | 389 | QRKFSRSDH | (SEQ ID NO:172) | 20.000 |
| 14 | 263 | GQSNHSTGY | (SEQ ID NO:100) | 20.000 |
| 15 | 416 | CRWPSCQKK | (SEQ ID NO:50) | 20.000 |
| 16 | 191 | QQYSVPPPV | (SEQ ID NO:171) | 10.000 |
| 17 | 217 | LRTPYSSDN | (SEQ ID NO:140) | 10.000 |
| 18 | 107 | CRYGPFGPP | (SEQ ID NO:51) | 10.000 |
| 19 | 98 | GQFTGTAGA | (SEQ ID NO:99) | 10.000 |
| 20 | 239 | NQMNLGATL | (SEQ ID NO:151) | 6.000 |

TABLE XIX
Results of BIMAS HLA Peptide Binding Prediction Analysis for
Binding of Human WT1 Peptides to Human HLA B2705

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 332 | KRYFKLSHL | (SEQ ID NO:127) | 30000.000 |
| 2 | 362 | RRFSRSDQL | (SEQ ID NO:187) | 30000.000 |
| 3 | 416 | CRWPSCQKK | (SEQ ID NO:50) | 10000.000 |
| 4 | 439 | QRNMTKLQL | (SEQ ID NO:173) | 2000.000 |
| 5 | 286 | YRIHTHGVF | (SEQ ID NO:252) | 1000.000 |
| 6 | 125 | ARMFPNAPY | (SEQ ID NO:38) | 1000.000 |
| 7 | 294 | FRGIQDVRR | (SEQ ID NO:81) | 1000.000 |
| 8 | 432 | VRHHNMHQR | (SEQ ID NO:243) | 1000.000 |
| 9 | 169 | AQFPNHSFK | (SEQ ID NO:36) | 1000.000 |
| 10 | 375 | RRHTGVKPF | (SEQ ID NO:188) | 900.000 |
| 11 | 126 | RMFPNAPYL | (SEQ ID NO:185) | 750.000 |
| 12 | 144 | IRNQGYSTV | (SEQ ID NO:117) | 600.000 |
| 13 | 301 | RRVPGVAPT | (SEQ ID NO:189) | 600.000 |
| 14 | 32 | AQWAPVLDF | (SEQ ID NO:37) | 500.000 |
| 15 | 191 | QQYSVPPPV | (SEQ ID NO:171) | 300.000 |
| 16 | 373 | HQRRHTGVK | (SEQ ID NO:109) | 200.000 |
| 17 | 426 | ARSDELVRH | (SEQ ID NO:39) | 200.000 |
| 18 | 383 | FQCKTCQRK | (SEQ ID NO:80) | 200.000 |
| 19 | 239 | NQMNLGATL | (SEQ ID NO:151) | 200.000 |
| 20 | 389 | QRKFSRSDH | (SEQ ID NO:172) | 200.000 |

TABLE XX
Results of BIMAS HLA Peptide Binding Prediction Analysis for
Binding of Human WT1 Peptides to Human HLA B3501

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 278 | TPILCGAQY | (SEQ ID NO:227) | 40.000 |
| 2 | 141 | QPAIRNQGY | (SEQ ID NO:170) | 40.000 |
| 3 | 219 | TPYSSDNLY | (SEQ ID NO:231) | 40.000 |
| 4 | 327 | YPGCNKRYF | (SEQ ID NO:250) | 20.000 |
| 5 | 163 | TPSHHAAQF | (SEQ ID NO:228) | 20.000 |
| 6 | 180 | DPMGQQGSL | (SEQ ID NO:59) | 20.000 |
| 7 | 221 | YSSDNLYQM | (SEQ ID NO:253) | 20.000 |
| 8 | 26 | LPVSGAAQW | (SEQ ID NO:138) | 10.000 |
| 9 | 174 | HSFKHEDPM | (SEQ ID NO:110) | 10.000 |
| 10 | 82 | EPHEEQCLS | (SEQ ID NO:68) | 6.000 |
| 11 | 213 | QALLLRTPY | (SEQ ID NO:160) | 6.000 |
| 12 | 119 | QASSGQARM | (SEQ ID NO:161) | 6.000 |
| 13 | 4 | DVRDLNALL | (SEQ ID NO:62) | 6.000 |
| 14 | 40 | FAPPGASAY | (SEQ ID NO:74) | 6.000 |
| 15 | 120 | ASSGQARMF | (SEQ ID NO:40) | 5.000 |
| 16 | 207 | DSCTGSQAL | (SEQ ID NO:61) | 5.000 |
| 17 | 303 | VPGVAPTLV | (SEQ ID NO:242) | 4.000 |
| 18 | 316 | ETSEKRPFM | (SEQ ID NO:73) | 4.000 |
| 19 | 152 | VTFDGTPSY | (SEQ ID NO:244) | 4.000 |
| 20 | 412 | KPFSCRWPS | (SEQ ID NO:123) | 4.000 |

TABLE XXI

Results of BIMAS HLA Peptide Binding Prediction Analysis for
Binding of Human WT1 Peptides to Human HLA B 3701

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 3 | SDVRDLNAL | (SEQ ID NO:206) | 40.000 |
| 2 | 273 | SDNHTTPIL | (SEQ ID NO:204) | 40.000 |
| 3 | 81 | AEPHEEQCL | (SEQ ID NO:30) | 10.000 |
| 4 | 298 | QDVRRVPGV | (SEQ ID NO:164) | 8.000 |
| 5 | 428 | SDELVRHHN | (SEQ ID NO:203) | 6.000 |
| 6 | 85 | EEQCLSAFT | (SEQ ID NO:65) | 5.000 |
| 7 | 208 | SCTGSQALL | (SEQ ID NO:202) | 5.000 |
| 8 | 4 | DVRDLNALL | (SEQ ID NO:62) | 5.000 |
| 9 | 209 | CTGSQALLL | (SEQ ID NO:52) | 5.000 |
| 10 | 38 | LDFAPPGAS | (SEQ ID NO:130) | 4.000 |
| 11 | 223 | SDNLYQMTS | (SEQ ID NO:205) | 4.000 |
| 12 | 179 | EDPMGQQGS | (SEQ ID NO:64) | 4.000 |
| 13 | 206 | TDSCTGSQA | (SEQ ID NO:220) | 4.000 |
| 14 | 6 | RDLNALLPA | (SEQ ID NO:177) | 4.000 |
| 15 | 84 | HEEQCLSAF | (SEQ ID NO:107) | 2.000 |
| 16 | 233 | LECMTWNQM | (SEQ ID NO:131) | 2.000 |
| 17 | 429 | DELVRHHNM | (SEQ ID NO:53) | 2.000 |
| 18 | 315 | SETSEKRPF | (SEQ ID NO:209) | 2.000 |
| 19 | 349 | GEKPYQCDF | (SEQ ID NO:91) | 2.000 |
| 20 | 302 | RVPGVAPTL | (SEQ ID NO:195) | 1.500 |

TABLE XXII

Results of BIMAS HLA Peptide Binding Prediction Analysis for
Binding of Human WT1 Peptides to Human HLA B 3801

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 437 | MHQRNMTKL | (SEQ ID NO:143) | 36.000 |
| 2 | 434 | HHNMHQRNM | (SEQ ID NO:108) | 6.000 |
| 3 | 372 | RHQRRHTGV | (SEQ ID NO:181) | 6.000 |
| 4 | 180 | DPMGQQGSL | (SEQ ID NO:59) | 4.000 |
| 5 | 433 | RHHNMHQRN | (SEQ ID NO:180) | 3.900 |
| 6 | 165 | SHHAAQFPN | (SEQ ID NO:213) | 3.900 |
| 7 | 202 | CHTPTDSCT | (SEQ ID NO:45) | 3.000 |
| 8 | 396 | DHLKTHTRT | (SEQ ID NO:57) | 3.000 |
| 9 | 161 | GHTPSHHAA | (SEQ ID NO:94) | 3.000 |
| 10 | 302 | RVPGVAPTL | (SEQ ID NO:195) | 2.600 |
| 11 | 417 | RWPSCQKKF | (SEQ ID NO:196) | 2.400 |
| 12 | 327 | YPGCNKRYF | (SEQ ID NO:250) | 2.400 |
| 13 | 208 | SCTGSQALL | (SEQ ID NO:202) | 2.000 |
| 14 | 163 | TPSHHAAQF | (SEQ ID NO:228) | 2.000 |
| 15 | 120 | ASSGQARMF | (SEQ ID NO:40) | 2.000 |
| 16 | 18 | LGGGGGCAL | (SEQ ID NO:134) | 2.000 |
| 17 | 177 | KHEDPMGQQ | (SEQ ID NO:121) | 1.800 |
| 18 | 83 | PHEEQCLSA | (SEQ ID NO:154) | 1.800 |
| 19 | 10 | ALLPAVPSL | (SEQ ID NO:34) | 1.300 |
| 20 | 225 | NLYQMTSQL | (SEQ ID NO:147) | 1.300 |

TABLE XXIII

Results of BIMAS HLA Peptide Binding Prediction Analysis for
Binding of Human WT1 Peptides to Human HLA B 3901

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 437 | MHQRNMTKL | (SEQ ID NO:143) | 135.000 |
| 2 | 332 | KRYFKLSHL | (SEQ ID NO:127) | 45.000 |
| 3 | 434 | HHNMHQRNM | (SEQ ID NO:108) | 30.000 |
| 4 | 362 | RRFSRSDQL | (SEQ ID NO:187) | 30.000 |
| 5 | 372 | RHQRRHTGV | (SEQ ID NO:181) | 30.000 |
| 6 | 10 | ALLPAVPSL | (SEQ ID NO:34) | 9.000 |
| 7 | 439 | QRNMTKLQL | (SEQ ID NO:173) | 7.500 |
| 8 | 390 | RKFSRSDHL | (SEQ ID NO:183) | 6.000 |
| 9 | 396 | DHLKTHTRT | (SEQ ID NO:57) | 6.000 |
| 10 | 239 | NQMNLGATL | (SEQ ID NO:151) | 6.000 |
| 11 | 423 | KKFARSDEL | (SEQ ID NO:122) | 6.000 |
| 12 | 126 | RMFPNAPYL | (SEQ ID NO:185) | 6.000 |
| 13 | 225 | NLYQMTSQL | (SEQ ID NO:147) | 6.000 |
| 14 | 180 | DPMGQQGSL | (SEQ ID NO:59) | 6.000 |
| 15 | 144 | IRNQGYSTV | (SEQ ID NO:117) | 5.000 |
| 16 | 136 | SCLESQPAI | (SEQ ID NO:198) | 4.000 |
| 17 | 292 | GVFRGIQDV | (SEQ ID NO:103) | 3.000 |
| 18 | 302 | RVPGVAPTL | (SEQ ID NO:195) | 3.000 |
| 19 | 208 | SCTGSQALL | (SEQ ID NO:202) | 3.000 |
| 20 | 207 | DSCTGSQAL | (SEQ ID NO:61) | 3.000 |

TABLE XXIV

Results of BIMAS HLA Peptide Bindina Prediction Analysis for
Binding of Human WT1 Peptides to Human HLA B 3902

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 239 | NQMNLGATL | (SEQ ID NO:151) | 24.000 |
| 2 | 390 | RKFSRSDHL | (SEQ ID NO:183) | 20.000 |
| 3 | 423 | KKFARSDEL | (SEQ ID NO:122) | 20.000 |
| 4 | 32 | AQWAPVLDF | (SEQ ID NO:37) | 5.000 |
| 5 | 146 | NQGYSTVTF | (SEQ ID NO:150) | 5.000 |
| 6 | 130 | NAPYLPSCL | (SEQ ID NO:144) | 2.400 |
| 7 | 225 | NLYQMTSQL | (SEQ ID NO:147) | 2.400 |
| 8 | 30 | GAAQWAPVL | (SEQ ID NO:86) | 2.400 |
| 9 | 441 | NMTKLQLAL | (SEQ ID NO:149) | 2.400 |
| 10 | 302 | RVPGVAPTL | (SEQ ID NO:195) | 2.400 |
| 11 | 126 | RMFPNAPYL | (SEQ ID NO:185) | 2.000 |
| 12 | 218 | RTPYSSDNL | (SEQ ID NO:194) | 2.000 |
| 13 | 209 | CTGSQALLL | (SEQ ID NO:52) | 2.000 |
| 14 | 332 | KRYFKLSHL | (SEQ ID NO:127) | 2.000 |
| 15 | 180 | DPMGQQGSL | (SEQ ID NO:59) | 2.000 |
| 16 | 437 | MHQRNMTKL | (SEQ ID NO:143) | 2.000 |
| 17 | 207 | DSCTGSQAL | (SEQ ID NO:61) | 2.000 |
| 18 | 208 | SCTGSQALL | (SEQ ID NO:202) | 2.000 |
| 19 | 329 | GCNKRYFKL | (SEQ ID NO:90) | 2.000 |
| 20 | 10 | ALLPAVPSL | (SEQ ID NO:34) | 2.000 |

TABLE XXV

Results of BIMAS HLA Peptide Binding Prediction Analysis for
Binding of Human WTI Peptides to Human HLA B 4403

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 315 | SETSEKRPF | (SEQ ID NO:209) | 80.000 |
| 2 | 349 | GEKPYQCDF | (SEQ ID NO:91) | 80.000 |
| 3 | 84 | HEEQCLSAF | (SEQ ID NO:107) | 60.000 |
| 4 | 410 | SEKPFSCRW | (SEQ ID NO:207) | 48.000 |
| 5 | 429 | DELVRHHNM | (SEQ ID NO:53) | 24.000 |
| 6 | 278 | TPILCGAQY | (SEQ ID NO:227) | 15.000 |
| 7 | 141 | QPAIRNQGY | (SEQ ID NO:170) | 9.000 |
| 8 | 40 | FAPPGASAY | (SEQ ID NO:74) | 9.000 |
| 9 | 213 | QALLLRTPY | (SEQ ID NO:160) | 9.000 |
| 10 | 318 | SEKRPFMCA | (SEQ ID NO:208) | 8.000 |
| 11 | 81 | AEPHEEQCL | (SEQ ID NO:30) | 8.000 |
| 12 | 152 | VTFDGTPSY | (SEQ ID NO:244) | 4.500 |
| 13 | 101 | TGTAGACRY | (SEQ ID NO:224) | 4.500 |
| 14 | 120 | ASSGQARMF | (SEQ ID NO:40) | 4.500 |
| 15 | 261 | TEGQSNHST | (SEQ ID NO:221) | 4.000 |
| 16 | 85 | EEQCLSAFT | (SEQ ID NO:65) | 4.000 |
| 17 | 233 | LECMTWNQM | (SEQ ID NO:131) | 4.000 |
| 18 | 104 | AGACRYGPF | (SEQ ID NO:31) | 4.000 |
| 19 | 3 | SDVRDLNAL | (SEQ ID NO:206) | 3.000 |
| 20 | 185 | QGSLGEQQY | (SEQ ID NO:166) | 3.000 |

TABLE XXVI

Results of BIMAS HLA Peptide Binding Prediction Analysis for
Binding of Human WT1 Peptides to Human HLA B 5101

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 303 | VPGVAPTLV | (SEQ ID NO:242) | 314.600 |
| 2 | 180 | DPMGQQGSL | (SEQ ID NO:59) | 242.000 |
| 3 | 250 | VAAGSSSSV | (SEQ ID NO:236) | 157.300 |
| 4 | 130 | NAPYLPSCL | (SEQ ID NO:144) | 50.000 |
| 5 | 30 | GAAQWAPVL | (SEQ ID NO:86) | 50.000 |
| 6 | 20 | GGGGCALPV | (SEQ ID NO:92) | 44.000 |
| 7 | 64 | PPPPPHSFI | (SEQ ID NO:157) | 40.000 |
| 8 | 29 | SGAAQWAPV | (SEQ ID NO:211) | 40.000 |
| 9 | 18 | LGGGGGCAL | (SEQ ID NO:134) | 31.460 |
| 10 | 295 | RGIQDVRRV | (SEQ ID NO:179) | 22.000 |
| 11 | 119 | QASSGQARM | (SEQ ID NO:161) | 18.150 |
| 12 | 418 | WPSCQKKFA | (SEQ ID NO:246) | 12.100 |
| 13 | 82 | EPHEEQCLS | (SEQ ID NO:68) | 12.100 |
| 14 | 110 | GPFGPPPPS | (SEQ ID NO:96) | 11.000 |
| 15 | 272 | ESDNHTTPI | (SEQ ID NO:71) | 8.000 |
| 16 | 306 | VAPTLVRSA | (SEQ ID NO:237) | 7.150 |
| 17 | 280 | ILCGAQYRI | (SEQ ID NO:116) | 6.921 |
| 18 | 219 | TPYSSDNLY | (SEQ ID NO:231) | 6.600 |
| 19 | 128 | FPNAPYLPS | (SEQ ID NO:79) | 6.500 |
| 20 | 204 | TPTDSCTGS | (SEQ ID NO:230) | 6.050 |

TABLE XXVII

Results of BIMAS HLA Peptide Binding Prediction Analysis for
Binding of Human WT1 Peptides to Human HLA B 5102

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 295 | RGIQDVRRV | (SEQ ID NO:179) | 290.400 |
| 2 | 303 | VPGVAPTLV | (SEQ ID NO:242) | 200.000 |
| 3 | 180 | DPMGQQGSL | (SEQ ID NO:59) | 133.100 |
| 4 | 250 | VAAGSSSSV | (SEQ ID NO:236) | 110.000 |
| 5 | 30 | GAAQWAPVL | (SEQ ID NO:86) | 55.000 |
| 6 | 130 | NAPYLPSCL | (SEQ ID NO:144) | 50.000 |
| 7 | 20 | GGGGCALPV | (SEQ ID NO:92) | 44.000 |
| 8 | 29 | SGAAQWAPV | (SEQ ID NO:211) | 44.000 |
| 9 | 64 | PPPPPHSFI | (SEQ ID NO:157) | 40.000 |
| 10 | 119 | QASSGQARM | (SEQ ID NO:161) | 36.300 |
| 11 | 110 | GPFGPPPPS | (SEQ ID NO:96) | 27.500 |
| 12 | 412 | KPFSCRWPS | (SEQ ID NO:123) | 25.000 |
| 13 | 18 | LGGGGGCAL | (SEQ ID NO:134) | 24.200 |
| 14 | 24 | CALPVSGAA | (SEQ ID NO:43) | 16.500 |
| 15 | 219 | TPYSSDNLY | (SEQ ID NO:231) | 15.000 |
| 16 | 292 | GVFRGIQDV | (SEQ ID NO:103) | 14.641 |
| 17 | 136 | SCLESQPAI | (SEQ ID NO:198) | 14.520 |
| 18 | 418 | WPSCQKKFA | (SEQ ID NO:246) | 12.100 |
| 19 | 269 | TGYESDNHT | (SEQ ID NO:225) | 11.000 |
| 20 | 351 | KPYQCDFKD | (SEQ ID NO:124) | 11.000 |

TABLE XXVIII

Results of BIMAS HLA Peptide Binding Prediction Analysis for
Binding of Human WT1 Peptides to Human HLA B 5201

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 191 | QQYSVPPPV | (SEQ ID NO:171) | 100.000 |
| 2 | 32 | AQWAPVLDF | (SEQ ID NO:37) | 30.000 |
| 3 | 243 | LGATLKGVA | (SEQ ID NO:133) | 16.500 |
| 4 | 303 | VPGVAPTLV | (SEQ ID NO:242) | 13.500 |
| 5 | 86 | EQCLSAFTV | (SEQ ID NO:69) | 12.000 |
| 6 | 295 | RGIQDVRRV | (SEQ ID NO:179) | 10.000 |
| 7 | 98 | GQFTGTAGA | (SEQ ID NO:99) | 8.250 |
| 8 | 292 | GVFRGIQDV | (SEQ ID NO:103) | 8.250 |
| 9 | 29 | SGAAQWAPV | (SEQ ID NO:211) | 6.000 |
| 10 | 146 | NQGYSTVTF | (SEQ ID NO:150) | 5.500 |
| 11 | 20 | GGGGCALPV | (SEQ ID NO:92) | 5.000 |
| 12 | 239 | NQMNLGATL | (SEQ ID NO:151) | 4.000 |
| 13 | 64 | PPPPPHSFI | (SEQ ID NO:157) | 3.600 |
| 14 | 273 | SDNHTTPIL | (SEQ ID NO:204) | 3.300 |
| 15 | 286 | YRIHTHGVF | (SEQ ID NO:252) | 3.000 |
| 16 | 269 | TGYESDNHT | (SEQ ID NO:225) | 3.000 |
| 17 | 406 | TGKTSEKPF | (SEQ ID NO:222) | 2.750 |
| 18 | 327 | YPGCNKRYF | (SEQ ID NO:250) | 2.750 |
| 19 | 7 | DLNALLPAV | (SEQ ID NO:58) | 2.640 |
| 20 | 104 | AGACRYGPF | (SEQ ID NO:31) | 2.500 |

TABLE XXIX
Results of BIMAS HLA Peptide Binding Prediction Analysis for
Binding of Human WT1 Peptides to Human HLA B 5801

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 230 | TSQLECMTW | (SEQ ID NO:234) | 96.800 |
| 2 | 92 | FTVHFSGQF | (SEQ ID NO:85) | 60.000 |
| 3 | 120 | ASSGQARMF | (SEQ ID NO:40) | 40.000 |
| 4 | 168 | AAQFPNHSF | (SEQ ID NO:29) | 20.000 |
| 5 | 408 | KTSEKPFSC | (SEQ ID NO:129) | 12.000 |
| 6 | 394 | RSDHLKTHT | (SEQ ID NO:192) | 9.900 |
| 7 | 276 | HTTPILCGA | (SEQ ID NO:115) | 7.200 |
| 8 | 218 | RTPYSSDNL | (SEQ ID NO:194) | 6.600 |
| 9 | 152 | VTFDGTPSY | (SEQ ID NO:244) | 6.000 |
| 10 | 40 | FAPPGASAY | (SEQ ID NO:74) | 6.000 |
| 11 | 213 | QALLLRTPY | (SEQ ID NO:160) | 4.500 |
| 12 | 347 | HTGEKPYQC | (SEQ ID NO:112) | 4.400 |
| 13 | 252 | AGSSSSVKW | (SEQ ID NO:32) | 4.400 |
| 14 | 211 | GSQALLLRT | (SEQ ID NO:102) | 4.356 |
| 15 | 174 | HSFKHEDPM | (SEQ ID NO:110) | 4.000 |
| 16 | 317 | TSEKRPFMC | (SEQ ID NO:233) | 4.000 |
| 17 | 26 | LPVSGAAQW | (SEQ ID NO:138) | 4.000 |
| 18 | 289 | HTHGVFRGI | (SEQ ID NO:113) | 3.600 |
| 19 | 222 | SSDNLYQMT | (SEQ ID NO:217) | 3.300 |
| 20 | 96 | FSGQFTGTA | (SEQ ID NO:82) | 3.300 |

TABLE XXX

Results of BIMAS HLA Peptide Binding Prediction Analysis for
Binding of Human WT1 Peptides to Human HLA CW0301

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 10 | ALLPAVPSL | (SEQ ID NO:34) | 100.000 |
| 2 | 332 | KRYFKLSHL | (SEQ ID NO:127) | 48.000 |
| 3 | 126 | RMFPNAPYL | (SEQ ID NO:185) | 36.000 |
| 4 | 3 | SDVRDLNAL | (SEQ ID NO:206) | 30.000 |
| 5 | 239 | NQMNLGATL | (SEQ ID NO:151) | 24.000 |
| 6 | 225 | NLYQMTSQL | (SEQ ID NO:147) | 24.000 |
| 7 | 180 | DPMGQQGSL | (SEQ ID NO:59) | 20.000 |
| 8 | 362 | RRFSRSDQL | (SEQ ID NO:187) | 12.000 |
| 9 | 329 | GCNKRYFKL | (SEQ ID NO:90) | 10.000 |
| 10 | 286 | YRIHTHGVF | (SEQ ID NO:252) | 10.000 |
| 11 | 301 | RRVPGVAPT | (SEQ ID NO:189) | 10.000 |
| 12 | 24 | CALPVSGAA | (SEQ ID NO:43) | 10.000 |
| 13 | 136 | SCLESQPAI | (SEQ ID NO:198) | 7.500 |
| 14 | 437 | MHQRNMTKL | (SEQ ID NO:143) | 7.200 |
| 15 | 390 | RKFSRSDHL | (SEQ ID NO:183) | 6.000 |
| 16 | 423 | KKFARSDEL | (SEQ ID NO:122) | 6.000 |
| 17 | 92 | FTVHFSGQF | (SEQ ID NO:85) | 5.000 |
| 18 | 429 | DELVRHHNM | (SEQ ID NO:53) | 5.000 |
| 19 | 130 | NAPYLPSCL | (SEQ ID NO:144) | 4.800 |
| 20 | 30 | GAAQWAPVL | (SEQ ID NO:86) | 4.000 |

TABLE XXXI

Results of BIMAS HLA Peptide Binding Prediction Analysis for
Binding of Human WT1 Peptides to Human HLA CW0401

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 356 | DFKDCERRF | (SEQ ID NO:55) | 120.000 |
| 2 | 334 | YFKLSHLQM | (SEQ ID NO:248) | 100.000 |
| 3 | 180 | DPMGQQGSL | (SEQ ID NO:59) | 88.000 |
| 4 | 163 | TPSHHAAQF | (SEQ ID NO:228) | 52.800 |
| 5 | 327 | YPGCNKRYF | (SEQ ID NO:250) | 40.000 |
| 6 | 285 | QYRIHTHGV | (SEQ ID NO:175) | 27.500 |
| 7 | 424 | KFARSDELV | (SEQ ID NO:119) | 25.000 |
| 8 | 326 | AYPGCNKRY | (SEQ ID NO:42) | 25.000 |
| 9 | 192 | QYSVPPPVY | (SEQ ID NO:176) | 25.000 |
| 10 | 417 | RWPSCQKKF | (SEQ ID NO:196) | 22.000 |
| 11 | 278 | TPILCGAQY | (SEQ ID NO:227) | 12.000 |
| 12 | 10 | ALLPAVPSL | (SEQ ID NO:34) | 11.616 |
| 13 | 141 | QPAIRNQGY | (SEQ ID NO:170) | 11.000 |
| 14 | 303 | VPGVAPTLV | (SEQ ID NO:242) | 11.000 |
| 15 | 219 | TPYSSDNLY | (SEQ ID NO:231) | 10.000 |
| 16 | 39 | DFAPPGASA | (SEQ ID NO:54) | 7.920 |
| 17 | 99 | QFTGTAGAC | (SEQ ID NO:165) | 6.000 |
| 18 | 4 | DVRDLNALL | (SEQ ID NO:62) | 5.760 |
| 19 | 70 | SFIKQEPSW | (SEQ ID NO:210) | 5.500 |
| 20 | 63 | PPPPPPHSF | (SEQ ID NO:158) | 5.280 |

TABLE XXXII
Results of BIMAS HLA Peptide Binding Prediction Analysis for
Binding of Human WT1 Peptides to Human HLA CW0602

| | | | | Score (Estimate of Half Time |
|---|---|---|---|---|
| | | | | of Disassociation of a |
| | Start | | | Molecule Containing This |
| Rank | Position | Subsequence Residue Listing | | Subsequence) |
| 1 | 332 | KRYFKLSHL | (SEQ ID NO:127) | 9.680 |
| 2 | 239 | NQMNLGATL | (SEQ ID NO:151) | 6.600 |
| 3 | 130 | NAPYLPSCL | (SEQ ID NO:144) | 6.600 |
| 4 | 7 | DLNALLPAV | (SEQ ID NO:58) | 6.000 |
| 5 | 441 | NMTKLQLAL | (SEQ ID NO:149) | 6.000 |
| 6 | 225 | NLYQMTSQL | (SEQ ID NO:147) | 6.000 |
| 7 | 4 | DVRDLNALL | (SEQ ID NO:62) | 6.000 |
| 8 | 3 | SDVRDLNAL | (SEQ ID NO:206) | 4.400 |
| 9 | 10 | ALLPAVPSL | (SEQ ID NO:34) | 4.000 |
| 10 | 213 | QALLLRTPY | (SEQ ID NO:160) | 3.300 |
| 11 | 319 | EKRPFMCAY | (SEQ ID NO:67) | 3.000 |
| 12 | 30 | GAAQWAPVL | (SEQ ID NO:86) | 2.200 |
| 13 | 242 | NLGATLKGV | (SEQ ID NO:146) | 2.200 |
| 14 | 292 | GVFRGIQDV | (SEQ ID NO:103) | 2.200 |
| 15 | 207 | DSCTGSQAL | (SEQ ID NO:61) | 2.200 |
| 16 | 362 | RRFSRSDQL | (SEQ ID NO:187) | 2.200 |
| 17 | 439 | QRNMTKLQL | (SEQ ID NO:173) | 2.200 |
| 18 | 295 | RGIQDVRRV | (SEQ ID NO:179) | 2.200 |
| 19 | 423 | KKFARSDEL | (SEQ ID NO:122) | 2.200 |
| 20 | 180 | DPMGQQGSL | (SEQ ID NO:59) | 2.200 |

TABLE XXXIII

Results of BIMAS HLA Peptide Binding Prediction Analysis for
Binding of Human WT1 Peptides to Human HLA CW0702

| | | | |
|---|---|---|---|
| | | | Score (Estimate of Half Time |
| | | | of Disassociation of a |
| | Start | | Molecule Containing his |
| Rank | Position | Subsequence Residue Listing | Subsequence) |

| | | | | |
|---|---|---|---|---|
| 1 | 319 | EKRPFMCAY | (SEQ ID NO:67) | 26.880 |
| 2 | 326 | AYPGCNKRY | (SEQ ID NO:42) | 24.000 |
| 3 | 40 | FAPPGASAY | (SEQ ID NO:74) | 14.784 |
| 4 | 192 | QYSVPPPVY | (SEQ ID NO:176) | 12.000 |
| 5 | 278 | TPILCGAQY | (SEQ ID NO:227) | 12.000 |
| 6 | 219 | TPYSSDNLY | (SEQ ID NO:231) | 12.000 |
| 7 | 213 | QALLLRTPY | (SEQ ID NO:160) | 8.800 |
| 8 | 125 | ARMFPNAPY | (SEQ ID NO:38) | 8.000 |
| 9 | 327 | YPGCNKRYF | (SEQ ID NO:250) | 6.600 |
| 10 | 152 | VTFDGTPSY | (SEQ ID NO:244) | 5.600 |
| 11 | 141 | QPAIRNQGY | (SEQ ID NO:170) | 4.800 |
| 12 | 345 | RKHTGEKPY | (SEQ ID NO:184) | 4.000 |
| 13 | 185 | QGSLGEQQY | (SEQ ID NO:166) | 4.000 |
| 14 | 101 | TGTAGACRY | (SEQ ID NO:224) | 4.000 |
| 15 | 375 | RRHTGVKPF | (SEQ ID NO:188) | 4.000 |
| 16 | 263 | GQSNHSTGY | (SEQ ID NO:100) | 4.000 |
| 17 | 163 | TPSHHAAQF | (SEQ ID NO:228) | 3.000 |
| 18 | 33 | QWAPVLDFA | (SEQ ID NO:174) | 2.688 |
| 19 | 130 | NAPYLPSCL | (SEQ ID NO:144) | 2.640 |
| 20 | 84 | HEEQCLSAF | (SEQ ID NO:107) | 2.400 |

TABLE XXXIV

Results of BIMAS HLA Peptide Binding Prediction Analysis for
Binding of Human WT1 Peptides to Mouse MHC Class I Db

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 235 | CMTWNQMNL | (SEQ ID NO:49) | 5255.712 |
| 2 | 126 | RMFPNAPYL | (SEQ ID NO:185) | 1990.800 |
| 3 | 221 | YSSDNLYQM | (SEQ ID NO:253) | 930.000 |
| 4 | 228 | QMTSQLECM | (SEQ ID NO:169) | 33.701 |
| 5 | 239 | NQMNLGATL | (SEQ ID NO:151) | 21.470 |
| 6 | 441 | NMTKLQLAL | (SEQ ID NO:149) | 19.908 |
| 7 | 437 | MHQRNMTKL | (SEQ ID NO:143) | 19.837 |
| 8 | 136 | SCLESQPAI | (SEQ ID NO:198) | 11.177 |
| 9 | 174 | HSFKHEDPM | (SEQ ID NO:110) | 10.800 |
| 10 | 302 | RVPGVAPTL | (SEQ ID NO:195) | 10.088 |
| 11 | 130 | NAPYLPSCL | (SEQ ID NO:144) | 8.400 |
| 12 | 10 | ALLPAVPSL | (SEQ ID NO:34) | 5.988 |
| 13 | 208 | SCTGSQALL | (SEQ ID NO:202) | 4.435 |
| 14 | 209 | CTGSQALLL | (SEQ ID NO:52) | 3.548 |
| 15 | 238 | WNQMNLGAT | (SEQ ID NO:245) | 3.300 |
| 16 | 218 | RTPYSSDNL | (SEQ ID NO:194) | 3.185 |
| 17 | 24 | CALPVSGAA | (SEQ ID NO:43) | 2.851 |
| 18 | 18 | LGGGGGCAL | (SEQ ID NO:134) | 2.177 |
| 19 | 142 | PAIRNQGYS | (SEQ ID NO:152) | 2.160 |
| 20 | 30 | GAAQWAPVL | (SEQ ID NO:86) | 1.680 |

TABLE XXXV
Results of BIMAS HLA Peptide Binding Prediction Analysis for
Binding of Human WT1 Peptides to Mouse MHC Class I Dd

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 112 | FGPPPPSQA | (SEQ ID NO:76) | 48.000 |
| 2 | 122 | SGQARMFPN | (SEQ ID NO:212) | 36.000 |
| 3 | 104 | AGACRYGPF | (SEQ ID NO:31) | 30.000 |
| 4 | 218 | RTPYSSDNL | (SEQ ID NO:194) | 28.800 |
| 5 | 130 | NAPYLPSCL | (SEQ ID NO:144) | 20.000 |
| 6 | 302 | RVPGVAPTL | (SEQ ID NO:195) | 20.000 |
| 7 | 18 | LGGGGGCAL | (SEQ ID NO:134) | 20.000 |
| 8 | 81 | AEPHEEQCL | (SEQ ID NO:30) | 10.000 |
| 9 | 29 | SGAAQWAPV | (SEQ ID NO:211) | 7.200 |
| 10 | 423 | KKFARSDEL | (SEQ ID NO:122) | 7.200 |
| 11 | 295 | RGIQDVRRV | (SEQ ID NO:179) | 7.200 |
| 12 | 390 | RKFSRSDHL | (SEQ ID NO:183) | 6.000 |
| 13 | 332 | KRYFKLSHL | (SEQ ID NO:127) | 6.000 |
| 14 | 362 | RRFSRSDQL | (SEQ ID NO:187) | 6.000 |
| 15 | 417 | RWPSCQKKF | (SEQ ID NO:196) | 6.000 |
| 16 | 160 | YGHTPSHHA | (SEQ ID NO:249) | 6.000 |
| 17 | 20 | GGGGCALPV | (SEQ ID NO:92) | 6.000 |
| 18 | 329 | GCNKRYFKL | (SEQ ID NO:90) | 5.000 |
| 19 | 372 | RHQRRHTGV | (SEQ ID NO:181) | 4.500 |
| 20 | 52 | GGPAPPPAP | (SEQ ID NO:93) | 4.000 |

TABLE XXXVI

Results of BIMAS HLA Peptide Binding Prediction Analysis for
Binding of Human WT1 Peptides to Mouse MHC Class I Kb

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 329 | GCNKRYFKL | (SEQ ID NO:90) | 24.000 |
| 2 | 225 | NLYQMTSQL | (SEQ ID NO:147) | 10.000 |
| 3 | 420 | SCQKKFARS | (SEQ ID NO:200) | 3.960 |
| 4 | 218 | RTPYSSDNL | (SEQ ID NO:194) | 3.630 |
| 5 | 437 | MHQRNMTKL | (SEQ ID NO:143) | 3.600 |
| 6 | 387 | TCQRKFSRS | (SEQ ID NO:219) | 3.600 |
| 7 | 302 | RVPGVAPTL | (SEQ ID NO:195) | 3.300 |
| 8 | 130 | NAPYLPSCL | (SEQ ID NO:144) | 3.000 |
| 9 | 289 | HTHGVFRGI | (SEQ ID NO:113) | 3.000 |
| 10 | 43 | PGASAYGSL | (SEQ ID NO:153) | 2.400 |
| 11 | 155 | DGTPSYGHT | (SEQ ID NO:56) | 2.400 |
| 12 | 273 | SDNHTTPIL | (SEQ ID NO:204) | 2.200 |
| 13 | 126 | RMFPNAPYL | (SEQ ID NO:185) | 2.200 |
| 14 | 128 | FPNAPYLPS | (SEQ ID NO:79) | 2.000 |
| 15 | 3 | SDVRDLNAL | (SEQ ID NO:206) | 1.584 |
| 16 | 207 | DSCTGSQAL | (SEQ ID NO:61) | 1.584 |
| 17 | 332 | KRYFKLSHL | (SEQ ID NO:127) | 1.500 |
| 18 | 18 | LGGGGGCAL | (SEQ ID NO:134) | 1.320 |
| 19 | 233 | LECMTWNQM | (SEQ ID NO:131) | 1.320 |
| 20 | 441 | NMTKLQLAL | (SEQ ID NO:149) | 1.200 |

TABLE XXXVII

Results of BIMAS HLA Peptide Binding Prediction Analysis for
Binding of Human WT1 Peptides to Mouse MHC Class I Kd

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 285 | QYRIHTHGV | (SEQ ID NO:175) | 600.000 |
| 2 | 424 | KFARSDELV | (SEQ ID NO:119) | 288.000 |
| 3 | 334 | YFKLSHLQM | (SEQ ID NO:248) | 120.000 |
| 4 | 136 | SCLESQPTI | (SEQ ID NO:199) | 115.200 |
| 5 | 239 | NQMNLGATL | (SEQ ID NO:151) | 115.200 |
| 6 | 10 | ALLPAVSSL | (SEQ ID NO:35) | 115.200 |
| 7 | 47 | AYGSLGGPA | (SEQ ID NO:41) | 86.400 |
| 8 | 180 | DPMGQQGSL | (SEQ ID NO:59) | 80.000 |
| 9 | 270 | GYESDNHTA | (SEQ ID NO:105) | 72.000 |
| 10 | 326 | AYPGCNKRY | (SEQ ID NO:42) | 60.000 |
| 11 | 192 | QYSVPPPVY | (SEQ ID NO:176) | 60.000 |
| 12 | 272 | ESDNHTAPI | (SEQ ID NO:70) | 57.600 |
| 13 | 289 | HTHGVFRGI | (SEQ ID NO:113) | 57.600 |
| 14 | 126 | DVRDLNALL | (SEQ ID NO:62) | 57.600 |
| 15 | 4 | CTGSQALLL | (SEQ ID NO:52) | 57.600 |
| 16 | 208 | SCTGSQALL | (SEQ ID NO:202) | 48.000 |
| 17 | 441 | NMTKLQLAL | (SEQ ID NO:149) | 48.000 |
| 18 | 207 | DSCTGSQAL | (SEQ ID NO:61) | 48.000 |
| 19 | 130 | NAPYLPSCL | (SEQ ID NO:144) | 48.000 |
| 20 | 235 | CMTWNQMNL | (SEQ ID NO:49) | 48.000 |

TABLE XXXVIII

Results of BIMAS HLA Peptide Binding Prediction Analysis for
Binding of Human WT1 Peptides to Mouse MHC Class I Kk

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 81 | AEPHEEQCL | (SEQ ID NO:30) | 40.000 |
| 2 | 85 | EEQCLSAFT | (SEQ ID NO:65) | 40.000 |
| 3 | 429 | DELVRHHNM | (SEQ ID NO:53) | 20.000 |
| 4 | 315 | SETSEKRPF | (SEQ ID NO:209) | 20.000 |
| 5 | 261 | TEGQSNHST | (SEQ ID NO:221) | 20.000 |
| 6 | 410 | SEKPFSCRW | (SEQ ID NO:207) | 10.000 |
| 7 | 272 | ESDNHTTPI | (SEQ ID NO:71) | 10.000 |
| 8 | 318 | SEKRPFMCA | (SEQ ID NO:208) | 10.000 |
| 9 | 138 | LESQPAIRN | (SEQ ID NO:132) | 10.000 |
| 10 | 233 | LECMTWNQM | (SEQ ID NO:131) | 10.000 |
| 11 | 298 | QDVRRVPGV | (SEQ ID NO:164) | 10.000 |
| 12 | 84 | HEEQCLSAF | (SEQ ID NO:107) | 10.000 |
| 13 | 349 | GEKPYQCDF | (SEQ ID NO:91) | 10.000 |
| 14 | 289 | HTHGVFRGI | (SEQ ID NO:113) | 10.000 |
| 15 | 179 | EDPMGQQGS | (SEQ ID NO:64) | 8.000 |
| 16 | 136 | SCLESQPAI | (SEQ ID NO:198) | 5.000 |
| 17 | 280 | ILCGAQYRI | (SEQ ID NO:116) | 5.000 |
| 18 | 273 | SDNHTTPIL | (SEQ ID NO:204) | 4.000 |
| 19 | 428 | SDELVRHHN | (SEQ ID NO:203) | 4.000 |
| 20 | 3 | SDVRDLNAL | (SEQ ID NO:206) | 4.000 |

TABLE XXXIX

Results of BIMAS HLA Peptide Binding Prediction Analysis for
Binding of Human WT1 Peptides to Mouse MHC Class I Ld

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 163 | TPSHHAAQF | (SEQ ID NO:228) | 360.000 |
| 2 | 327 | YPGCNKRYF | (SEQ ID NO:250) | 300.000 |
| 3 | 180 | DPMGQQGSL | (SEQ ID NO:59) | 150.000 |
| 4 | 26 | LPVSGAAQW | (SEQ ID NO:138) | 93.600 |
| 5 | 278 | TPILCGAQY | (SEQ ID NO:227) | 72.000 |
| 6 | 141 | QPAIRNQGY | (SEQ ID NO:170) | 60.000 |
| 7 | 219 | TPYSSDNLY | (SEQ ID NO:231) | 60.000 |
| 8 | 303 | VPGVAPTLV | (SEQ ID NO:242) | 60.000 |
| 9 | 120 | ASSGQARMF | (SEQ ID NO:40) | 50.000 |
| 10 | 63 | PPPPPPHSF | (SEQ ID NO:158) | 45.000 |
| 11 | 113 | GPPPPSQAS | (SEQ ID NO:97) | 45.000 |
| 12 | 157 | TPSYGHTPS | (SEQ ID NO:229) | 39.000 |
| 13 | 207 | DSCTGSQAL | (SEQ ID NO:61) | 32.500 |
| 14 | 110 | GPFGPPPPS | (SEQ ID NO:96) | 30.000 |
| 15 | 82 | EPHEEQCLS | (SEQ ID NO:68) | 30.000 |
| 16 | 412 | KPFSCRWPS | (SEQ ID NO:123) | 30.000 |
| 17 | 418 | WPSCQKKFA | (SEQ ID NO:246) | 30.000 |
| 18 | 221 | YSSDNLYQM | (SEQ ID NO:253) | 30.000 |
| 19 | 204 | TPTDSCTGS | (SEQ ID NO:230) | 30.000 |
| 20 | 128 | FPNAPYLPS | (SEQ ID NO:79) | 30.000 |

TABLE XL

Results of BIMAS HLA Peptide Binding Prediction Analysis for
Binding of Human WT1 Peptides to Cattle HLA A20

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 350 | EKPYQCDFK | (SEQ ID NO:66) | 1000.00 |
| 2 | 319 | EKRPFMCAY | (SEQ ID NO:67) | 500.000 |
| 3 | 423 | KKFARSDEL | (SEQ ID NO:122) | 500.000 |
| 4 | 345 | RKHTGEKPY | (SEQ ID NO:184) | 500.000 |
| 5 | 390 | RKFSRSDHL | (SEQ ID NO:183) | 500.000 |
| 6 | 137 | CLESQPAIR | (SEQ ID NO:47) | 120.000 |
| 7 | 380 | VKPFQCKTC | (SEQ ID NO:239) | 100.000 |
| 8 | 407 | GKTSEKPFS | (SEQ ID NO:95) | 100.000 |
| 9 | 335 | FKLSHLQMH | (SEQ ID NO:78) | 100.000 |
| 10 | 247 | LKGVAAGSS | (SEQ ID NO:135) | 100.000 |
| 11 | 370 | LKRHQRRHT | (SEQ ID NO:136) | 100.000 |
| 12 | 258 | VKWTEGQSN | (SEQ ID NO:240) | 100.000 |
| 13 | 398 | LKTHTRTHT | (SEQ ID NO:137) | 100.000 |
| 14 | 331 | NKRYFKLSH | (SEQ ID NO:145) | 100.000 |
| 15 | 357 | FKDCERRFS | (SEQ ID NO:77) | 100.000 |
| 16 | 385 | CKTCQRKFS | (SEQ ID NO:46) | 100.000 |
| 17 | 294 | FRGIQDVRR | (SEQ ID NO:81) | 80.000 |
| 18 | 368 | DQLKRHQRR | (SEQ ID NO:60) | 80.000 |
| 19 | 432 | VRHHNMHQR | (SEQ ID NO:243) | 80.000 |
| 20 | 118 | SQASSGQAR | (SEQ ID NO:216) | 80.000 |

TABLE XLI

Results of BIMAS HLA Peptide Binding Prediction Analysis for
Binding of Mouse WT1 Peptides to Mouse MHC Class I A 0201

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 126 | RMFPNAPYL | (SEQ ID NO:293) | 313.968 |
| 2 | 187 | SLGEQQYSV | (SEQ ID NO:299) | 285.163 |
| 3 | 10 | ALLPAVSSL | (SEQ ID NO:255) | 181.794 |
| 4 | 225 | NLYQMTSQL | (SEQ ID NO:284) | 68.360 |
| 5 | 292 | GVFRGIQDV | (SEQ ID NO:270) | 51.790 |
| 6 | 93 | TLHFSGQFT | (SEQ ID NO:302) | 40.986 |
| 7 | 191 | QQYSVPPPV | (SEQ ID NO:290) | 22.566 |
| 8 | 280 | ILCGAQYRI | (SEQ ID NO:274) | 17.736 |
| 9 | 441 | NMTKLHVAL | (SEQ ID NO:285) | 15.428 |
| 10 | 235 | CMTWNQMNL | (SEQ ID NO:258) | 15.428 |
| 11 | 7 | DLNALLPAV | (SEQ ID NO:261) | 11.998 |
| 12 | 242 | NLGATLKGM | (SEQ ID NO:283) | 11.426 |
| 13 | 227 | YQMTSQLEC | (SEQ ID NO:307) | 8.573 |
| 14 | 239 | NQMNLGATL | (SEQ ID NO:286) | 8.014 |
| 15 | 309 | TLVRSASET | (SEQ ID NO:303) | 7.452 |
| 16 | 408 | KTSEKPFSC | (SEQ ID NO:277) | 5.743 |
| 17 | 340 | LQMHSRKHT | (SEQ ID NO:280) | 4.752 |
| 18 | 228 | QMTSQLECM | (SEQ ID NO:289) | 4.044 |
| 19 | 37 | VLDFAPPGA | (SEQ ID NO:304) | 3.378 |
| 20 | 302 | RVSGVAPTL | (SEQ ID NO:295) | 1.869 |

TABLE XLII

Results of BIMAS HLA Peptide Binding Prediction Analysis for
Binding of Mouse WT1 Peptides to Mouse MHC Class I Db

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 221 | YSSDNLYQM | (SEQ ID NO:308) | 312.000 |
| 2 | 126 | RMFPNAPYL | (SEQ ID NO:293) | 260.000 |
| 3 | 235 | CMTWNQMNL | (SEQ ID NO:258) | 260.000 |
| 4 | 437 | MHQRNMTKL | (SEQ ID NO:281) | 200.000 |
| 5 | 238 | WNQMNLGAT | (SEQ ID NO:305) | 12.000 |
| 6 | 130 | NAPYLPSCL | (SEQ ID NO:282) | 8.580 |
| 7 | 3 | SDVRDLNAL | (SEQ ID NO:298) | 7.920 |
| 8 | 136 | SCLESQPTI | (SEQ ID NO:296) | 7.920 |
| 9 | 81 | AEPHEEQCL | (SEQ ID NO:254) | 6.600 |
| 10 | 10 | ALLPAVSSL | (SEQ ID NO:255) | 6.600 |
| 11 | 218 | RTPYSSDNL | (SEQ ID NO:294) | 6.000 |
| 12 | 441 | NMTKLHVAL | (SEQ ID NO:285) | 3.432 |
| 13 | 228 | QMTSQLECM | (SEQ ID NO:289) | 3.120 |
| 14 | 174 | HSFKHEDPM | (SEQ ID NO:272) | 3.120 |
| 15 | 242 | NLGATLKGM | (SEQ ID NO:283) | 2.640 |
| 16 | 261 | TEGQSNHGI | (SEQ ID NO:301) | 2.640 |
| 17 | 225 | NLYQMTSQL | (SEQ ID NO:284) | 2.640 |
| 18 | 207 | DSCTGSQAL | (SEQ ID NO:263) | 2.600 |
| 19 | 119 | QASSGQARM | (SEQ ID NO:288) | 2.600 |
| 20 | 18 | LGGGGGCGL | (SEQ ID NO:279) | 2.600 |

TABLE XLIII

Results of BIMAS HLA Peptide Binding Prediction Analysis for
Binding of Mouse WT1 Peptides to Mouse MUC Class I Kb

| | | | |
|---|---|---|---|
| | | | +HZ,26 Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 329 | GCNKRYFKL | (SEQ ID NO:268) | 24.000 |
| 2 | 225 | NLYQMTSQL | (SEQ ID NO:284) | 10.000 |
| 3 | 420 | SCQKKFARS | (SEQ ID NO:297) | 3.960 |
| 4 | 218 | RTPYSSDNL | (SEQ ID NO:294) | 3.630 |
| 5 | 437 | MHQRNMTKL | (SEQ ID NO:281) | 3.600 |
| 6 | 387 | TCQRKFSRS | (SEQ ID NO:300) | 3.600 |
| 7 | 289 | HTHGVFRGI | (SEQ ID NO:273) | 3.000 |
| 8 | 130 | NAPYLPSCL | (SEQ ID NO:282) | 3.000 |
| 9 | 43 | PGASAYGSL | (SEQ ID NO:287) | 2.400 |
| 10 | 155 | DGAPSYGHT | (SEQ ID NO:260) | 2.400 |
| 11 | 126 | RMFPNAPYL | (SEQ ID NO:293) | 2.200 |
| 12 | 128 | FPNAPYLPS | (SEQ ID NO:267) | 2.000 |
| 13 | 207 | DSCTGSQAL | (SEQ ID NO:263) | 1.584 |
| 14 | 3 | SDVRDLNAL | (SEQ ID NO:298) | 1.584 |
| 15 | 332 | KRYFKLSHL | (SEQ ID NO:276) | 1.500 |
| 16 | 233 | LECMTWNQM | (SEQ ID NO:278) | 1.320 |
| 17 | 18 | LGGGGGCGL | (SEQ ID NO:279) | 1.320 |
| 18 | 242 | NLGATLKGM | (SEQ ID NO:283) | 1.200 |
| 19 | 123 | GQARMFPN | (SEQ ID NO:269)A | 1.200 |
| 20 | 441 | NMTKLHVAL | (SEQ ID NO:285) | 1.200 |

TABLE XLIV

Results of BIMAS HLA Peptide Binding Prediction Analysis for
Binding of Mouse WT1 Peptides to Mouse MHC Class I Kd

| | | | |
|---|---|---|---|
| | | | Score (Estimate of Half Time |
| | | | of Disassociation of a |
| | Start | | Molecule Containing This |
| Rank | Position | Subsequence Residue Listing | Subsequence) |

| | | | | |
|---|---|---|---|---|
| 1 | 285 | QYRIHTHGV | (SEQ ID NO:291) | 600.000 |
| 2 | 424 | KFARSDELV | (SEQ ID NO:275) | 288.000 |
| 3 | 334 | YFKLSHLQM | (SEQ ID NO:306) | 120.000 |
| 4 | 136 | SCLESQPTI | (SEQ ID NO:296) | 115.200 |
| 5 | 239 | NQMNLGATL | (SEQ ID NO:286) | 115.200 |
| 6 | 10 | ALLPAVSSL | (SEQ ID NO:255) | 115.200 |
| 7 | 47 | AYGSLGGPA | (SEQ ID NO:256) | 86.400 |
| 8 | 180 | DPMGQQGSL | (SEQ ID NO:262) | 80.000 |
| 9 | 270 | GYESDNHTA | (SEQ ID NO:271) | 72.000 |
| 10 | 192 | QYSVPPPVY | (SEQ ID NO:292) | 60.000 |
| 11 | 326 | AYPGCNKRY | (SEQ ID NO:257) | 60.000 |
| 12 | 289 | HTHGVFRGI | (SEQ ID NO:273) | 57.600 |
| 13 | 4 | DVRDLNALL | (SEQ ID NO:264) | 57.600 |
| 14 | 126 | RMFPNAPYL | (SEQ ID NO:293) | 57.600 |
| 15 | 209 | CTGSQALLL | (SEQ ID NO:259) | 48.000 |
| 16 | 86 | EQCLSAFTL | (SEQ ID NO:265) | 48.000 |
| 17 | 302 | RVSGVAPTL | (SEQ ID NO:295) | 48.000 |
| 18 | 218 | RTPYSSDNL | (SEQ ID NO:294) | 48.000 |
| 19 | 272 | ESDNHTAPI | (SEQ ID NO:266) | 48.000 |
| 20 | 225 | NLYQMTSQL | (SEQ ID NO:284) | 48.000 |

TABLE XLV
Results of TSites Peptide Binding Prediction Ana-
lysis for Human WT1 Peptides Capable of Eliciting
a Helper T cell Response

| Peptide | Sequence | |
|---|---|---|
| p6-23 | RDLNALLPAVPSLGGGG | (SEQ ID |
| | | NO:1) |
| p30-35 | GAAQWA | (SEQ ID |
| | | NO:309) |
| p45-56 | ASAYGSLGGPAP | (SEQ ID |
| | | NO:310) |
| p91-105 | AFTVHFSGQFTGTAG | (SEQ ID |
| | | NO:311) |
| p117-139 | PSQASSGQARMFPNAPYLPSCLE | (SEQ ID |
| | | NO:2) |
| p167-171 | HAAQF | (SEQ ID |
| | | NO:312) |
| p202-233 | CHTPTDSCTGSQALLLRTPYSSDNLYQMTSQL | (SEQ ID |
| | | NO:313) |
| p244-262 | GATLKGVAAGSSSSVKWTE | (SEQ ID |
| | | NO:4) |
| p287-318 | RIHTHGVFRGIQDVRRVPGVAPTLVRSASETS | (SEQ ID |
| | | NO:314) |
| p333-336 | RYFK | (SEQ ID |
| | | NO:315) |
| p361-374 | ERRFSRSDQLKRHQ | (SEQ ID |
| | | NO:316) |
| p389-410 | QRKFSRSDHLKTHTRTHTGKTS | (SEQ ID |
| | | NO:317) |
| p421-441 | CQKKFARSDELVRHHNMHQRN | (SEQ ID |
| | | NO:318) |

Certain CTL peptides (shown in Table XLVI) were selected for further study. For each peptide in Table XLVI, scores obtained using BIMAS HLA peptide binding prediction analysis are provided.

TABLE XLVI

WT1 Peptide Sequences and HLA Peptide Binding Predictions

| | | |
|---|---|---|
| Peptide | Sequence | Comments |

| | | | |
|---|---|---|---|
| p329-337 | GCNKRYFKL | SEQ ID NOs: 90 and 268) | Score 24,000 |
| p225-233 | NLYQMTSQL | (SEQ ID NOs:147 and 284) | binds also to class II and HLA A2, Kd, score 10,000 |
| p235-243 | CMTWNQMNL | (SEQ ID NOs:49 and 258) | binds also to HLA A2, score 5,255,712 |
| p126-134 | RMFPNAPYL | (SEQ ID NOs:185 and 293) | binds also to Kd, class II and HLA A2, score |
| | | | 1,990,800 |
| p221-229 | YSSDNLYQM | (SEQ ID NOs:253 and 308) | binds also to Ld, score 312,000 |
| p228-236 | QMTSQLECM | (SEQ ID NOs:169 and 289) | score 3,120 |
| p239-247 | NQMNLGATL | (SEQ ID NOs:151 and 286) | binds also to HLA A 0201, Kd, score 8,015 |
| mouse p136-144 | SCLESQPTI | (SEQ ID NO:296) | binds also to Kd, 1mismatch to human |
| human p136-144 | SCLESQPAI | (SEQ ID NO:198) | score 7,920 |
| mouse p10-18 | ALLPAVSSL | (SEQ ID NO:255) | binds also to Kd, HLA A2, 1 mismatch to human |
| human p10-18 | ALLPAVPSL | (SEQ ID NO:34) | score 6,600 |

Peptide binding to C57B1/6 murine MHC was confirmed using the leukemia cell line RMA-S, as described by Ljunggren et al., Nature 346:476-480, 1990. In brief, RMA-S cells were cultured for 7 hours at 26° C. in complete medium supplemented with 1% FCS. A total of 10⁶ RMA-S cells were added into each well of a 24-well plate and incubated either alone or with the designated peptide (25 ug/ml) for 16 hours at 26° C. and additional 3 hours at 37° C. in complete medium. Cells were then washed three times and stained with fluorescein isothiocyanate-conjugated anti D^{b} or anti-K^{b} antibody (PharMingen, San Diego, Calif.). Labeled cells were washed twice, resuspended and fixed in 500 ul of PBS with 1% paraformaldehyde and analyzed for fluorescence intensity in a flow cytometer (Becton-Dickinson FACSCalibur®). The percentage of increase of D^{b} or K^{b} molecules on the surface of the RMA-S cells was measured by increased mean fluorescent intensity of cells incubated with peptide compared with that of cells incubated in medium alone.

Mice were immunized with the peptides capable of binding to murine class I MHC. Following immunization, spleen cells were stimulated in vitro and tested for the ability to lyse targets incubated with WT1 peptides. CTL were evaluated with a standard chromium release assay (Chen et al., Cancer Res. 54:1065-1070, 1994). 10⁶ target cells were incubated at 37° C. with 150μCi of sodium ⁵¹Cr for 90 minutes, in the presence or absence of specific peptides. Cells were washed three times and resuspended in RPMI with 5% fetal bovine serum. For the assay, 10^{4 51}Cr-labeled target cells were incubated with different concentrations of effector cells in a final volume of 200μl in U-bottomed 96-well plates. Supernatants were removed after 4 to 7 hours at 37° C., and the percentage specific lysis was determined by the formula:

% specific lysis=100×(experimental release−spontaneous release)/(maximum release−spontaneous release).

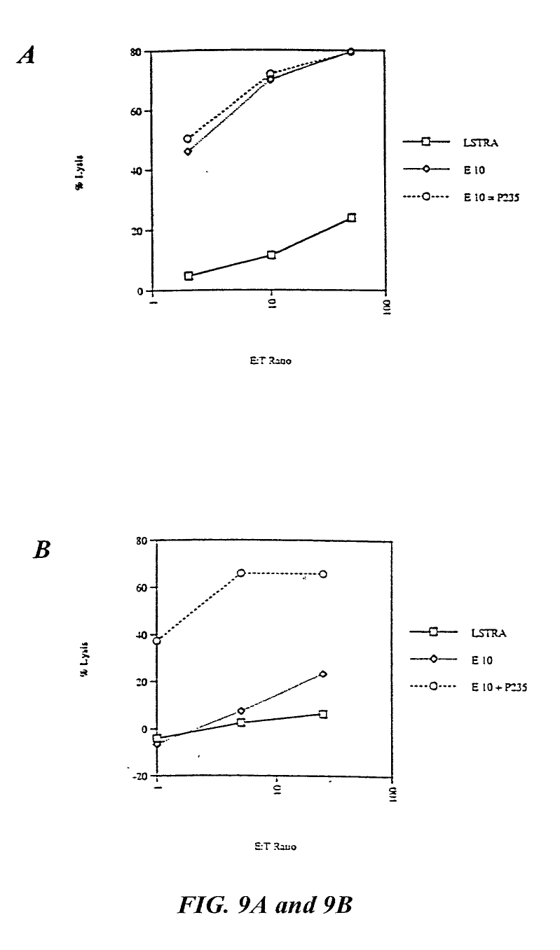

The results, presented in Table XLVII, show that some WT1 peptides can bind to class I MHC molecules, which is essential for generating CTL. Moreover, several of the peptides were able to elicit peptide specific CTL (FIGS. 9A and 9B), as determined using chromium release assays. Following immunization to CTL peptides p10-18 human, p136-144 human, p136-144 mouse and p235-243, peptide specific CTL lines were generated and clones were established. These results indicate that peptide specific CTL can kill malignant cells expressing WT1.

TABLE XLVII
Binding of WT1 CTL Peptides to mouse B6 class I
antigens

| | Binding Affinity to Mouse MHC Class |
|---|---|
| Peptide | I |
| Positive control | 91% |
| negative control | 0.5.-1.3% |
| p235-243 | 33.6% |
| p136-144 mouse | 27.9% |
| p136-144 human | 52% |
| p10-18: human | 2.2% |
| p225-233 | 5.8% |
| p329-337 | 1.2% |
| p126-134 | 0.9% |
| p221-229 | 0.8% |
| p228-236 | 1.2% |
| p239-247 | 1% |

### Example 5

### Use of a WT1 Polypeptide to Elicit WT1 Specific CTL in Mice

This Example illustrates the ability of a representative WT1 polypeptide to elicit CTL immunity capable of killing WT1 positive tumor cell lines.

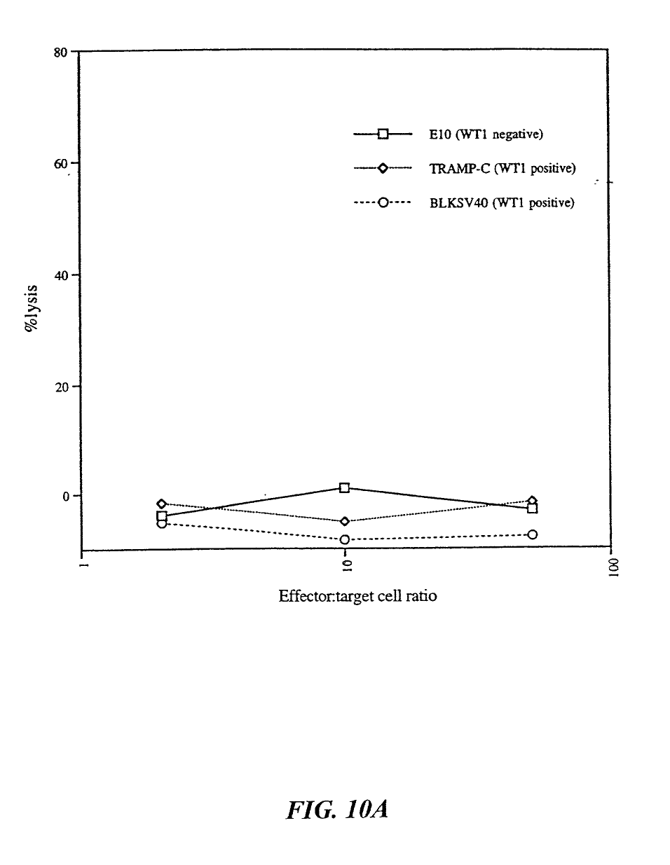

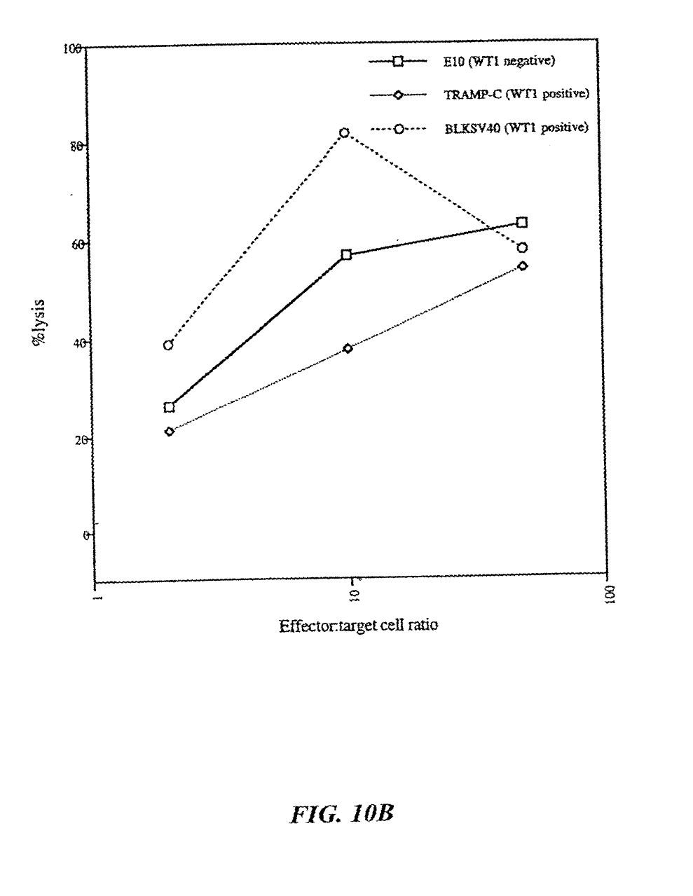

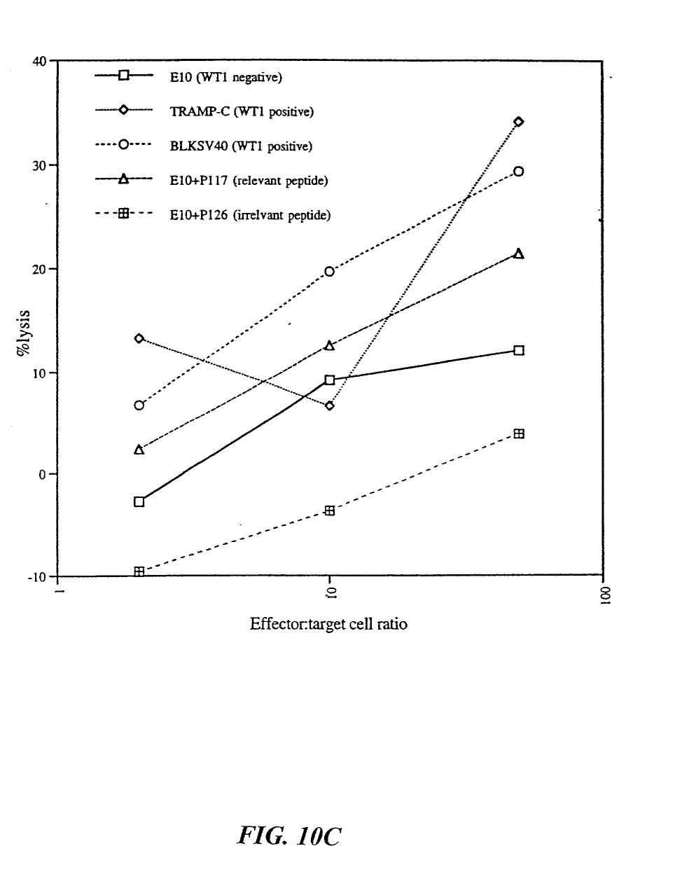

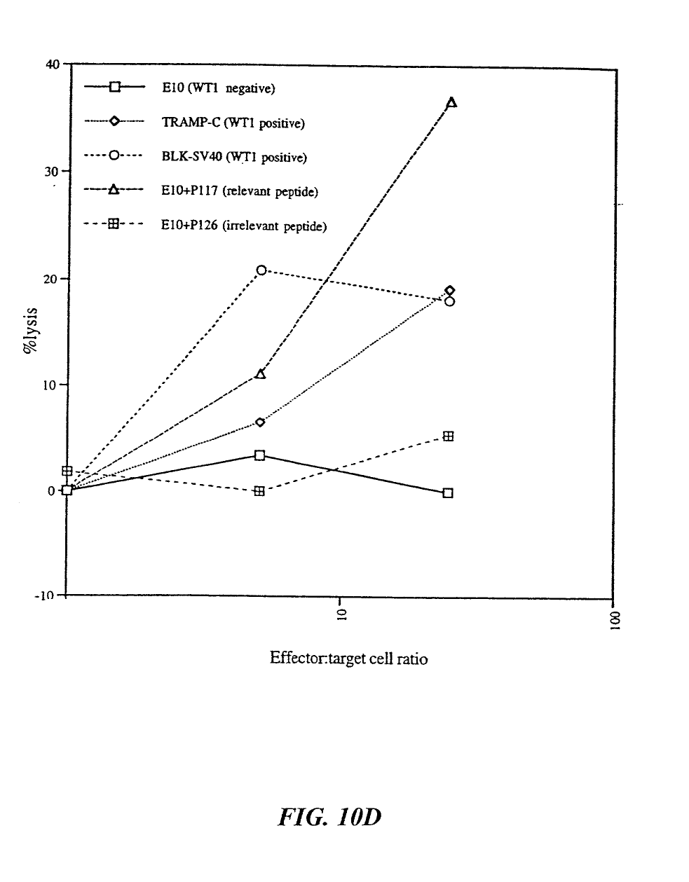

P117-139, a peptide with motifs appropriate for binding to class I and class II MHC, was identified as described above using TSITES and BIMAS HLA peptide binding prediction analyses. Mice were immunized as described in Example 3. Following immunization, spleen cells were stimulated in vitro and tested for the ability to lyse targets incubated with WT1 peptides, as well as WT1 positive and negative tumor cells. CTL were evaluated with a standard chromium release assay. The results, presented in FIGS. 10A-10D, show that P117 can elicit WT1 specific CTL capable of killing WT1 positive tumor cells, whereas no killing of WT1 negative cells was observed. These results demonstrate that peptide specific CTL in fact kill malignant cells expressing WT1 and that vaccine and T cell therapy are effective against malignancies that express WT1.

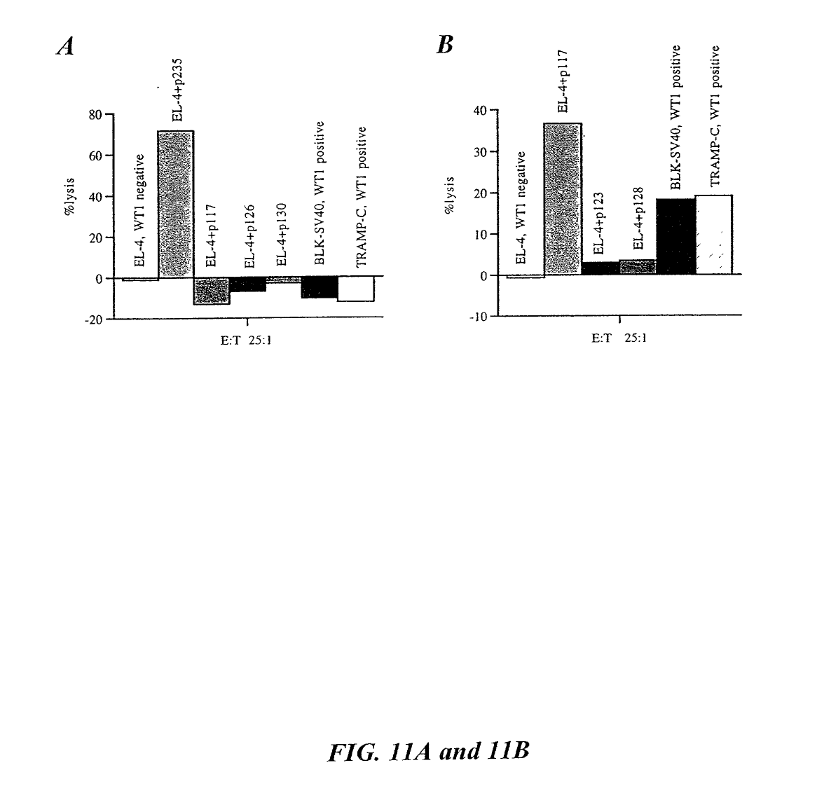

Similar immunizations were performed using the 9-mer class I MHC binding peptides p136-144, p225-233, p235-243 as well as the 23-mer peptide p117-139. Following immunization, spleen cells were stimulated in vitro with each of the 4 peptides and tested for ability to lyse targets incubated with WT1 peptides. CTL were generated specific for p136-144, p235-243 and pl 17-139, but not for p225-233. CTL data for p235-243 and p117-139 are presented in FIGS. 11A and 11B. Data for peptides p136-144 and p225-233 are not depicted.

CTL lysis demands that the target WT1 peptides are endogenously processed and presented in association with tumor cell class I MHC molecules. The above WT1 peptide specific CTL were tested for ability to lyse WT1 positive versus negative tumor cell lines. CTL specific for p235-243 lysed targets incubated with the p235-243 peptides, but failed to lyse cell lines that expressed WT1 proteins (FIG. 11A). By marked contrast, CTL specific for p117-139 lysed targets incubated with p117-139 peptides and also lysed malignant cells expressing WT1 (FIG. 11B). As a negative control, CTL specific for p117-139 did not lyse WT1 negative EL-4 (also referred to herein as E10).

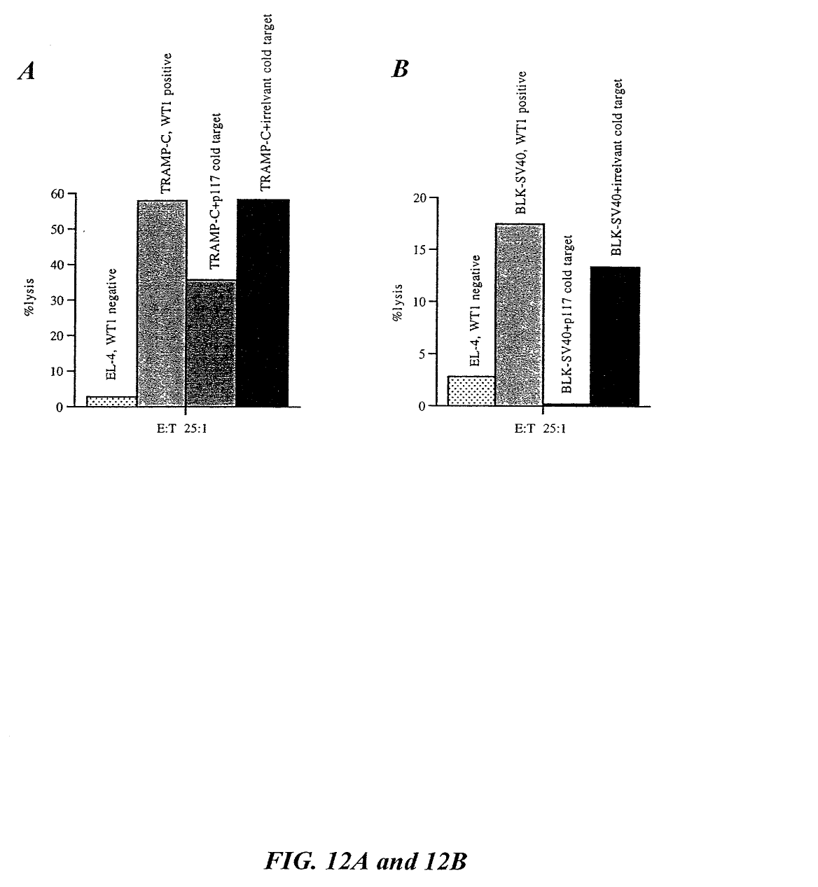

Specificity of WT1 specific lysis was confirmed by cold target inhibition (FIGS. 12A-12B). Effector cells were plated for various effector: target ratios in 96-well U-bottom plates. A ten-fold excess (compared to hot target) of the indicated peptide-coated target without ⁵¹Cr labeling was added. Finally, 10^{4 51}Cr-labeled target cells per well were added and the plates incubated at 37° C. for 4 hours. The total volume per well was 200μl.

Lysis of TRAMP-C by p117-139 specific CTL was blocked from 58% to 36% by EL-4 incubated with the relevant peptide p117-139, but not with EL-4 incubated with an irrelevant peptide (FIG. 12A). Similarly, lysis of BLK-SV40 was blocked from 18% to 0% by EL-4 incubated with the relevant peptide p117-139 (FIG. 12B). Results validate that WT1 peptide specific CTL specifically kill malignant cells by recognition of processed WT1.

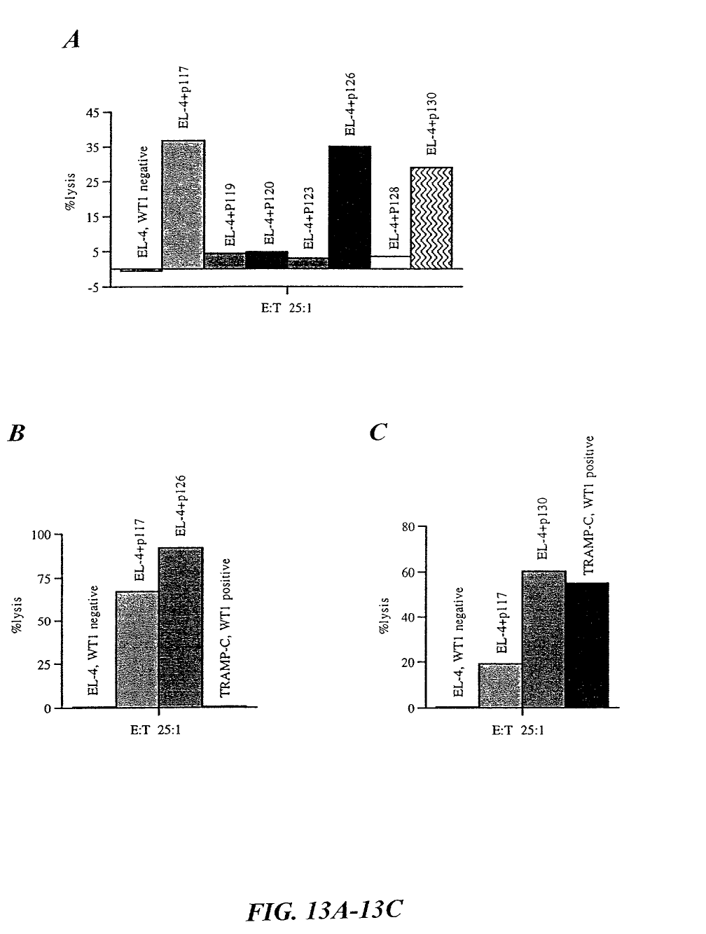

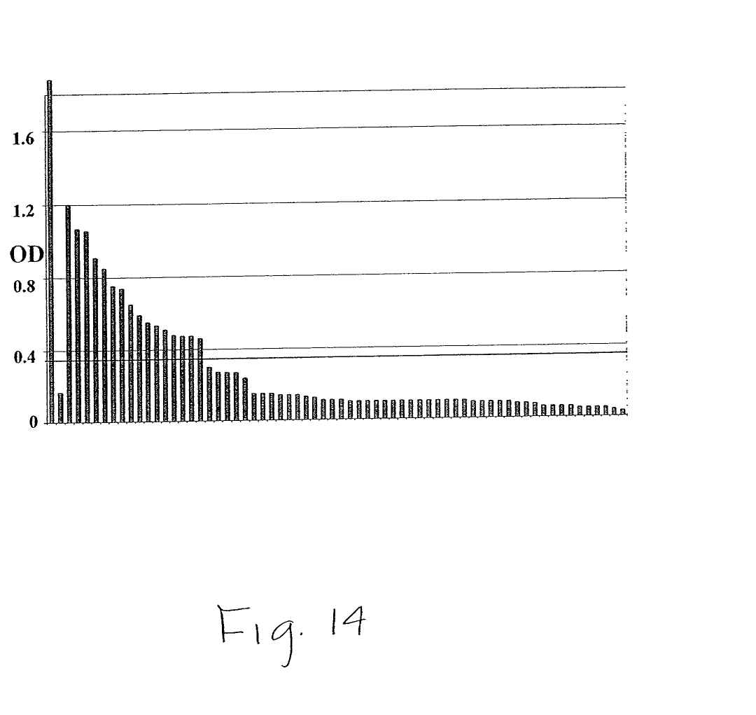

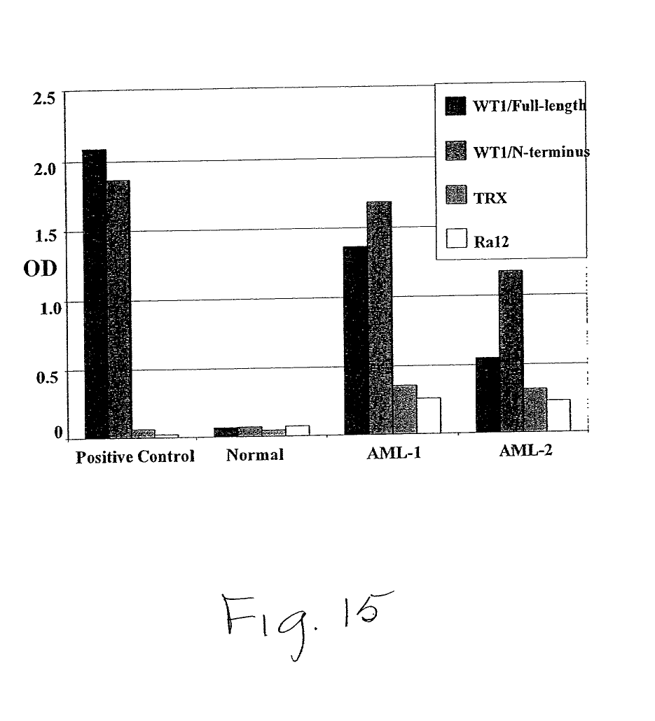

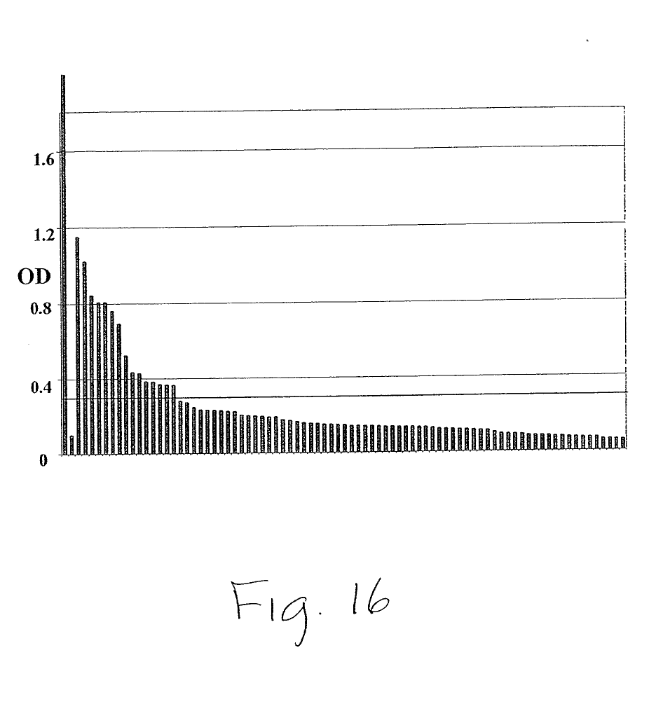

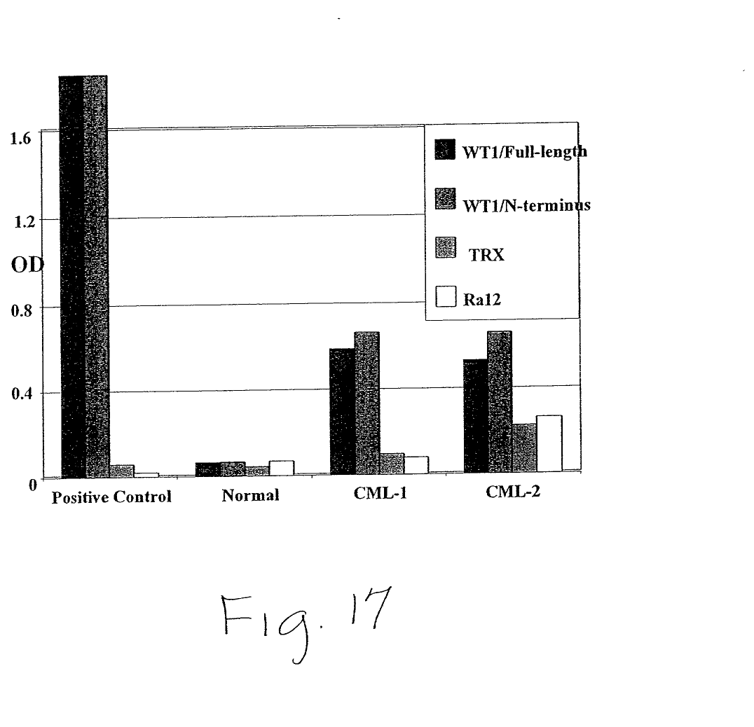

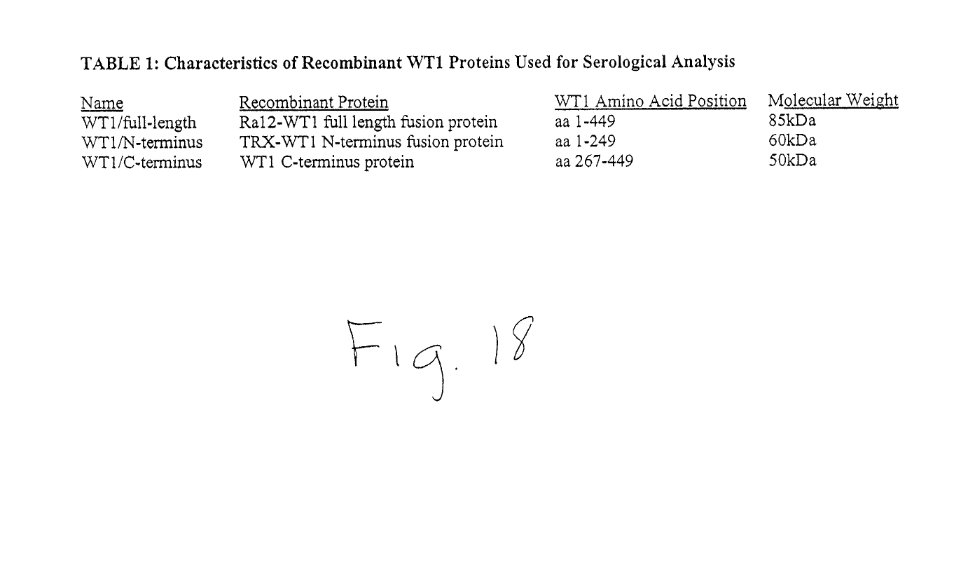

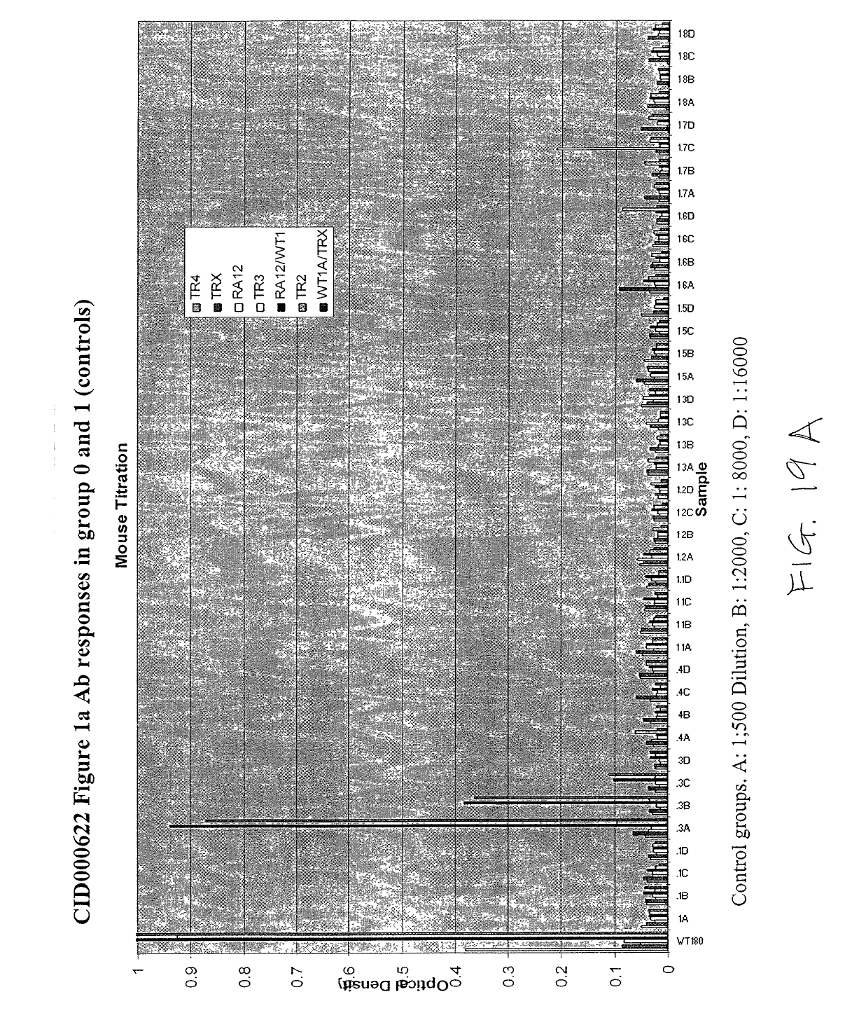

Several segments with putative CTL motifs are contained within p117-139. To determine the precise sequence of the CTL epitope all potential 9-mer peptides within p117-139 were synthesized (Table XLVIII). Two of these peptides (p126-134 and p130-138) were shown to bind to H-2^{b} class I molecules (Table XLVIII). CTL generated by immunization with p117-139 lysed targets incubated with p126-134 and p130-138, but not the other 9-mer peptides within p117-139 (FIG. 13A).

The p117-139 specific CTL line was restimulated with either p126-134 or p130-138. Following restimulation with p126-134 or p130-138, both T cell lines demonstrated peptide specific lysis, but only p130-138 specific CTL showed lysis of a WT1 positive tumor cell line (FIGS. 13B and 13C). Thus, p130-138 appears to be the naturally processed epitope.

TABLE XLVIII

Binding of WT1 CTL 9mer Peptides within p117-139 to mouse B6
class I antigens Binding Affinity to

| | |
|---|---|
| Peptide | Mouse MHC Class I |

| | | | |
|---|---|---|---|
| P117-125 | PSQASSGQA | (SEQ ID NO:221) | 2% |
| P118-126 | SQASSGQAR | (SEQ ID NO:216) | 2% |
| P119-127 | QASSGQARM | (SEQ ID Nos: 161 and 288) | 2% |
| P120-128 | ASSGQARMF | (SEQ ID NO:40 | 1% |
| P121-129 | SSGQARMFP | (SEQ ID NO:222) | 1% |
| P122-130 | SGQARMFPN | (SEQ ID NO:212) | 1% |
| P123-131 | GQARMFPNA | (SEQ ID Nos: 98 and 269) | 1% |
| P124-132 | QARMFPNAP | (SEQ ID NO:223) | 1% |
| P125-133 | ARMFPNAPY | (SEQ ID NO:38) | 1% |
| P126-134 | RMFPNAPYL | (SEQ ID NOs: 185 and 293) | 79% |
| P127-135 | MFPNAPYLP | (SEQ ID NO:224) | 2% |
| P128-136 | FPNAPYLPS | (SEQ ID NOs: 79 and 267) | 1% |
| P129-137 | PNAPYLPSC | (SEQ ID NO:225) | 1% |
| P130-138 | NAPYLPSCL | (SEQ ID NOs: 144 and 282) | 79% |
| P131-139 | APYLPSCLE | (SEQ ID NO:226) | 1% |

### Example 6

### Identification of WT1 Specific mRNA in Mouse Tumor Cell Lines

This Example illustrates the use of RT-PCR to detect WT1 specific mRNA in cells and cell lines.

Mononuclear cells were isolated by density gradient centrifugation, and were immediately frozen and stored at −80° C. until analyzed by RT-PCR for the presence of WT1 specific mRNA. RT-PCR was generally performed as described by Fraizer et al., Blood 86:4704-4706, 1995. Total RNA was extracted from 10⁷ cells according to standard procedures. RNA pellets were resuspended in 25 μL diethylpyrocarbonate treated water and used directly for reverse transcription. The zinc-finger region (exons 7 to 10) was amplified by PCR as a 330 bp mouse cDNA. Amplification was performed in a thermocycler during one or, when necessary, two sequential rounds of PCR. AmpliTaq DNA Polymerase (Perkin Elmer Cetus, Norwalk, Conn.), 2.5 mM MgCl₂ and 20 pmol of each primer in a total reaction volume of 50 μl were used. Twenty μL aliquots of the PCR products were electrophoresed on 2% agarose gels stained with ethidium bromide. The gels were photographed with Polaroid film (Polaroid 667, Polaroid Ltd., Hertfordshire, England). Precautions against cross contamination were taken following the recommendations of Kwok and Higuchi, Nature 339:237-238, 1989. Negative controls included the cDNA- and PCR-reagent mixes with water instead of cDNA in each experiment. To avoid false negatives, the presence of intact RNA and adequate cDNA generation was evaluated for each sample by a control PCR using β-actin primers. Samples that did not amplify with these primers were excluded from analysis.

Primers for amplification of WT1 in mouse cell lines were: P115: 1458-1478: 5′ CCC AGG CTG CAA TAA GAG ATA 3′ (forward primer; SEQ ID NO:21); and P116: 1767-1787: 5′ ATG TTG TGA TGG CGG ACC AAT 3′ (reverse primer; SEQ ID NO:22) (see Inoue et al, Blood 88:2267-2278, 1996; Fraizer et al., Blood 86:4704-4706, 1995).

Beta Actin primers used in the control reactions were: 5′ GTG GGG CGC CCC AGG CAC CA 3′ (sense primer; SEQ ID NO:23); and 5′ GTC CTT AAT GTC ACG CAC GAT TTC 3′ (antisense primer; SEQ ID NO:24) Primers for use in amplifying human WT1 include: P117: 954-974: 5′ GGC ATC TGA GAC CAG TGA GAA 3′ (SEQ ID NO:25); and P118: 1434-1414: 5′ GAG AGT CAG ACT TGA AAG CAGT 3′ (SEQ ID NO:5). For nested RT-PCR, primers may be: P119: 1023-1043: 5′ GCT GTC CCA CTT ACA GAT GCA 3′ (SEQ ID NO:26); and P120: 1345-1365: 5′ TCA AAG CGC CAG CTG GAG TTT 3′ (SEQ ID NO:27).

Table XLVIII shows the results of WT1 PCR analysis of mouse tumor cell lines. Within Table IV, (+++) indicates a strong WT1 PCR amplification product in the first step RT PCR, (++) indicates a WT1 amplification product that is detectable by first step WT1 RT PCR, (+) indicates a product that is detectable only in the second step of WT1 RT PCR, and (−) indicates WT1 PCR negative.

TABLE XLIX
Detection of WT1 mRNA in Mouse Tumor Cell Lines

| | WT1 |
|---|---|
| Cell Line | mRNA |
| K562 (human leukemia; ATCC): Positive control; (Lozzio and | +++ |
| Lozzio, Blood 45: 321-334, 1975) | |
| TRAMPC (SV40 transformed prostate, B6); Foster et al., Cancer | +++ |
| Res. 57: 3325-3330, 1997 | |
| BLK-SV40 HD2 (SV40-transf. fibroblast, B6; ATCC); Nature | ++ |
| 276: 510-511, 1978 | |
| CTLL (T-cell, B6; ATCC); Gillis, Nature 268: 154-156, 1977) | + |
| FM (FBL-3 subline, leukemia, B6); Glynn and Fefer, Cancer | + |
| Res. 28: 434-439, 1968 | |
| BALB 3T3 (ATCC); Aaroston and Todaro, J. Cell. Physiol. | + |
| 72: 141-148, 1968 | |
| S49.1 (Lymphoma, T-cell like, B/C; ATCC); Horibata and | + |
| Harris, Exp. Cell. Res. 60: 61, 1970 | |
| BNL CL.2 (embryonic liver, B/C; ATCC); Nature 276: 510-511, | + |
| 1978 | |
| MethA (sarcoma, B/C); Old et al., Ann. NY Acad. Sci. 101: 80- | − |
| 106, 1962 | |
| P3.6.2.8.1 (myeloma, B/C; ATCC); Proc. Natl. Acad. Sci. USA | − |
| 66: 344, 1970 | |
| P2N (leukemia, DBA/2; ATCC); Melling et al., J. Immunol. | − |
| 117: 1267-1274, 1976 | |
| BCL1 (lymphoma, B/C; ATCC); Slavin and Strober, Nature | − |
| 272: 624-626, 1977 | |
| LSTRA (lymphoma, B/C); Glynn et al., Cancer Res. 28: 434-439, | − |
| 1968 | |
| E10/EL-4 (lymphoma, B6); Glynn et al., Cancer Res. 28: 434- | − |
| 439, 1968 | |

### Example 7

### Expression in E. coli of WT1 Trx Fusion Construct

The truncated open reading frame of WT1 (WT1B) was PCR amplified with the following primers:

Forward Primer starting at amino acid 2

| | |
|---|---|
| P-37 (SEQ ID NO.342) | 5′ ggctccgacgtgcgggacctg 3′ Tm 64° C. |

Reverse Primer creating EcoRI site after stop codon

| | |
|---|---|
| P-23 (SEQ ID NO.343) | 5′ gaattctcaaagcgccagctggagtttggt 3′ Tm 63° C. |

The PCR was performed under the following conditions:

10 μl 10X Pfu buffer
1 μl 10 mM dNTPs
2 μl 10 μM each oligo
83 μL sterile water
1.5 μl Pfu DNA polymerase (Stratagene, La Jolla, CA)
50 ng DNA (pPDM FL WT1)

| | | |
|---|---|---|
| 96° C. 2 minutes | | |
| 96° C. 20 seconds | 63° C. 15 seconds | 72° C. 3 minutes × |
| | | 40 cycles |
| 72° C. 4 minutes | | |

The PCR product was digested with EcoRI restriction enzyme, gel purified and then cloned into pTrx 2H vector (a modified pET28 vector with a Trx fusion on the N-terminal and two His tags surrounding the Trx fusion. After the Trx fusion there exists protease cleavage sites for thrombin and enterokinase). The pTrx2H construct was digested with StuI and EcoRI restriction enzymes. The correct constructs were confirmed by DNA sequence analysis and then transformed into BL21 (DE3) pLys S and BL21 (DE3) CodonPlus expression host cells.

### Example 8

### Expression in E. coli of WT1 A His Tag Fusion Constructs

The N-terminal open reading frame of WT1 (WT1A) was PCR amplified with the following primers:

Forward Primer starting at amino acid 2

| | |
|---|---|
| P-37 (SEQ ID NO.344) | 5′ ggctccgacgtgcgggacctg 3′ Tm 64° C. |

Reverse Primer creating EcoRI site after an artificial stop codon
put after amino acid 249.

| | |
|---|---|
| PDM-335 (SEQ ID NO.345) | 5′ gaattctcaaagcgccagctggagtttggt 3′ Tm 64° C. |

The PCR was performed under the following conditions:

10 μl 10X Pfu buffer
1 μl 10 mM dNTPs
2 μl 10 μM each oligo
83 μL sterile water
1.5 μl Pfu DNA polymerase (Stratagene, La Jolla, CA)
50 ng DNA (pPDM FL WT1)

| | | |
|---|---|---|
| 96° C. 2 minutes | | |
| 96° C. 20 seconds | 63° C. 15 seconds | 72° C. 1 minute |
| | | 20 seconds × 40 cycles |
| 72° C. 4 minutes | | |

The PCR product was digested with EcoRI restriction enzyme, gel purified and then cloned into pPDM, a modified pET28 vector with a His tag in frame, which had been digested with Eco72I and EcoRI restriction enzymes. The PCR product was also transformed into pTrx 2H vector. The pTrx2H construct was digested with StuI and EcoRI restriction enzymes. The correct constructs were confirmed by DNA sequence analysis and then transformed into BL21 (DE3) pLys S and BL21 (DE3) CodonPlus expression host cells.

### Example 9

### Expression in E. coli of WT1 B His Tag Fusion Constructs

The truncated open reading frame of WT1 (WT1A) was PCR amplified with the following primers:

Forward Primer starting at amino acid 250

| | |
|---|---|
| PDM-346 (SEQ ID NO. 346) | 5′ cacagcacagggtacgagagc 3′ Tm 58° C. |

Reverse Primer creating EcoRiL site after stop codon

| | |
|---|---|
| P-23 (SEQ ID NO. 347) | 5′ gaattctcaaagcgccagctggagtttggt 3′ Tm 63° C. |

The PCR was performed under the following conditions:

10 μl 10X Pfu buffer
1 μl 10 mM dNTPs
2 μl 10 μM each oligo
83 μL sterile water
1.5 μl Pfu DNA polymerase (Stratagene, La Jolla, CA)
50 ng DNA (pPDM FL WT1)

| | | |
|---|---|---|
| 96° C. 2 minutes | | |
| 96° C. 20 seconds | 63° C. 15 seconds | 72° C. 1 minute |
| | | 30 seconds × 40 cycles |
| 72° C. 4 minutes | | |

The PCR product was digested with EcoRI restriction enzyme, gel purified and then cloned into pPDM, a modified pET28 vector with a His tag in frame, which had been digested with Eco72I and EcoRI restriction enzymes. The PCR product was also transformed into pTrx 2H vector. The pTrx 2H construct was digested with StuI and EcoRI restriction enzymes. The correct constructs were confirmed by DNA sequence analysis and then transformed into BL21 (DE3) pLys S and BL21 (DE3) CodonPlus expression host cells.

For Examples 7-9, the following SEQ ID NOs. are disclosed:

SEQ ID NO. 327 is the determined cDNA sequence for Trx_WT1_B

SEQ ID NO. 328 is the determined cDNA sequence for Trx_WT1_A

SEQ ID NO. 329 is the determined cDNA sequence for Trx_WT1

SEQ ID NO. 330 is the determined cDNA sequence for WT1_A

SEQ ID NO. 331 is the determined cDNA sequence for WT1_B

SEQ ID NO. 332 is the predicted amino acid sequence encoded by SEQ ID No. 327

SEQ ID NO. 333 is the predicted amino acid sequence encoded by SEQ ID No. 328

SEQ ID NO. 334 is the predicted amino acid sequence encoded by SEQ ID No. 329

SEQ ID NO. 335 is the predicted amino acid sequence encoded by SEQ ID No. 330

SEQ ID NO. 336 is the predicted amino acid sequence encoded by SEQ ID No. 331

### Example 10

### Truncated Forms of WT1 Expressed in E. coli

Three reading frames of WT1 were amplified by PCR using the following primers:

| | |
|---|---|
| For WT1 Tr2: | |
| PDM-441 (SEQ ID NO. 348) | 5′ cacgaagaacagtgcctgagcgcattcac 3′ Tm 63° C. |
| PDM-442 (SEQ ID NO. 349) | 5′ ccggcgaattcatcagtataaattgtcactgc 3′ TM 62° C. |
| For WT1 Tr3: | |
| PDM-443 (SEQ ID NO. 350) | 5′ caggctttgctgctgaggacgccc 3′ Tm 64° C. |
| PDM-444 (SEQ ID NO. 351) | 5′ cacggagaattcatcactggtatggtttctcacc Tm 64° C. |
| For WT1 Tr4: | |
| PDM-445 (SEQ ID NO. 352) | 5′ cacagcaggaagcacactggtgagaaac 3′ Tm 63° C. |
| PDM-446 (SEQ ID NO. 353) | 5′ ggatatctgcagaattctcaaagcgccagc 3′ TM 63° C. |

The PCR was performed under the following conditions:

10 μl 10X Pfu buffer
1 μl 10 mM dNTPs
2 μl 10 μM each oligo
83 μL sterile water
1.5 μl Pfu DNA polymerase (Stratagene, La Jolla, CA)
50 ng DNA (pPDM FL WT1)

| | | |
|---|---|---|
| 96° C. 2 minutes | | |
| 96° C. 20 seconds | 63° C. 15 seconds | 72° C. 30 seconds × |
| | | 40 cycles |
| 72° C. 4 minutes | | |

The PCR products were digested with EcoRI and cloned into pPDM His (a modified pET28 vector with a His tag in frame on the 5′ end) which has been digested with Eco72I and EcoRI. The constructs were confirmed to be correct through sequence analysis and transformed into BL21 pLys S and BL21 CodonPlus cells or BLR pLys S and BLR CodonPlus cells.

### Example 11

### WT1 C (amino acids 76-437) and WT1 D (amino acids 91-437) Expression in E. coli

The WT1 C reading frame was amplified by PCR using the following primers:

| | |
|---|---|
| PDM-504 (SEQ ID NO. 354) | 5′ cactccttcatcaaacaggaac 3′ Tm 61° C. |
| PDM-446 (SEQ ID NO. 355) | 5′ ggatatctgcagaattctcaaagcgccagc 3′ Tm 63° C. |

The PCR was performed under the following conditions:

10 μl 10X Pfu buffer
1 μl 10 mM dNTPs
2 μl 10 μM each oligo
83 μL sterile water
1.5 μl Pfu DNA polymerase (Stratagene, La Jolla, CA)
50 ng DNA (pPDM FL WT1)

| | | |
|---|---|---|
| 96° C. 2 minutes | | |
| 96° C. 20 seconds | 63° C. 15 seconds | 72° C. 2 minutes × |
| | | 40 cycles |
| 72° C. 4 minutes | | |

The PCR product was digested with EcoRI and cloned into pPDM His which had been digested with Eco72I and EcoRI. The sequence was confirmed through sequence analysis and then transformed into BLR pLys S and BLR which is co-transformed with CodonPlus RP.

### Example 12

### Synthetic Production of WT1 TR-1 by Annealing Overlapping Oligos

This example was performed to determine the effect of changing proline codon usage on expression.

The following pairs of oligos were annealed:

| | | |
|---|---|---|
| 1. | PDM-505 (SEQ ID NO. 356) | 5′ ggttccgacgtgcgggacctgaacgcactgctg 3′ |
| | PDM-506 (SEQ ID NO. 357) | 5′ ctgccggcagcagtgcgttcaggtcccgcacgtcggaacc 3′ |
| 2. | PDM-507 (SEQ ID NO. 358) | 5′ ccggcagttccatccctgggtggcggtggaggctg 3′ |
| | PDM-508 (SEQ ID NO. 359) | 5′ cggcagtgcgcagcctccaccgccacccagggatggaa 3′ |
| 3. | PDM-509 (SEQ ID NO. 360) | 5′ cgcactgccggttagcggtgcagcacagtgggctc 3′ |
| | PDM-510 (SEQ ID NO. 361) | 5′ cagaactggagcccactgtgctgcaccgctaac 3′ |
| 4. | PDM-511 (SEQ ID NO. 362) | 5′ cagttctggacttcgcaccgcctggtgcatccgcatac 3′ |
| | PDM-512 (SEQ ID NO. 363) | 5′ cagggaaccgtatgcggatgcaccaggcggtgcgaagtc 3′ |
| 5. | PDM-513 (SEQ ID NO. 364) | 5′ ggttccctgggtggtccagcacctccgcccgcaacgcc 3′ |
| | PDM-514 (SEQ ID NO. 365) | 5′ ggcggtgggggcgttgcgggcggaggtgctggaccacc 3′ |
| 6. | PDM-515 (SEQ ID NO. 366) | 5′ cccaccgcctccaccgcccccgcactccttcatcaaacag 3′ |
| | PDM-516 (SEQ ID NO. 367) | 5′ ctaggttcctgtttgatgaaggagtgcgggggcggtgga 3′ |
| 7. | PDM-517 (SEQ ID NO. 368) | 5′ gaacctagctggggtggtgcagaaccgcacgaagaaca 3′ |
| | PDM-518 (SEQ ID NO. 369) | 5′ ctcaggcactgttcttcgtgcggttctgcaccaccccag 3′ |
| 8. | PDM-519 (SEQ ID NO. 370) | 5′ gtgcctgagcgcattctgagaattctgcagat 3′ |
| | PDM-520 (SEQ ID NO. 371) | 5′ gtgtgatggatatctgcagaattctcagaatgcg 3′ |

Each oligo pair was separately combined then annealed. The pairs were then ligated together and one μl of ligation mix was used for PCR conditions below:

10 μl 10X Pfu buffer
1 μl 10 mM dNTPs
2 μl 10 μM each oligo
83 μL sterile water
1.5 μl Pfu DNA polymerase (Stratagene, La Jolla, CA)

| | | |
|---|---|---|
| 96° C. 2 minutes | | |
| 96° C. 20 seconds | 63° C. 15 seconds | 72° C. 30 seconds × |
| | | 40 cycles |
| 72° C. 4 minutes | | |

The PCR product was digested with EcoRI and cloned into pPDM His which had been digested with Eco72I and EcoRI. The sequence was confirmed and then transformed into BLR pLys S and BLR which is co-transformed with CodonPlus RP.

For examples 10-12, the following SEQ ID NOs. are disclosed:

SEQ ID NO:337 is the determined cDNA sequence for WT1_Tr1

SEQ ID NO:338 is the determined cDNA sequence for WT1_Tr2

SEQ ID NO:339 is the determined cDNA sequence for WT1_Tr3

SEQ ID NO:340 is the determined cDNA sequence for WT1_Tr4

SEQ ID NO:341 is the determined cDNA sequence for WT1_C

SEQ ID NO:342 is the predicted amino acid sequence encoded by SEQ ID NO:337

SEQ ID NO:343 is the predicted amino acid sequence encoded by SEQ ID NO:338

SEQ ID NO:344 is the predicted amino acid sequence encoded by SEQ ID NO:339

SEQ ID NO:345 is the predicted amino acid sequence encoded by SEQ ID NO:340

SEQ ID NO:346 is the predicted amino acid sequence encoded by SEQ ID NO:341

The WT1 C sequence represents a polynucleotide having the coding regions of TR2, TR3 and TR4.

The WT1 TR-1 synthetic sequence represents a polynucleotide in which alternative codons for proline were substituted for the native codons, producing a polynucleotide capable of expressing WT1 TR-1 in E. coli.

### Example 13

### Evaluation of the Systemic Histopathological and Toxicological Effects of WT1 Immunization in Mice

The purpose of this example is to analyze the immunogenicity and potential systemic histopathological and toxicological effects of WT1 protein immunization in a multiple dose titration in mice.

The experimental design for immunization of mice with WT1 protein is outlined in Table L.

TABLE L
Experimental Design of WT1 Immunization in Mice

| Histology | Corixa | | Dose | Total No. |
|---|---|---|---|---|
| Group | Group | Treatment Description | Level | (Females) |
| 1 | 0 | No treatment | 0 | 4 |
| 2 | 1.1 | MPL-SE (adjuvants alone), 6x, 1 week apart | 10 ug | 4 |
| 3 | 1.2 | MPL-SE, 3x, 2 weeks apart | 10 ug | 4 |
| 4 | 2.1 | Ra12-WT1 + MPL-SE, 6x | 25 ug | 4 |
| 5 | 2.2 | Ra12-WT1 + MPL-SE, 3x | 25 ug | 4 |
| 6 | 3.1 | Ra12-WT1 + MPL-SE, 6x | 100 ug | 4 |
| 7 | 3.2 | Ra12-WT1 + MPL-SE, 3x | 100 ug | 4 |
| 8 | 4.1 | Ra12-WT1 + MPL-SE, 6x | 1000 ug | 4 |
| 9 | 4.2 | Ra12-WT1 + MPL-SE, 3x | 1000 ug | 4 |

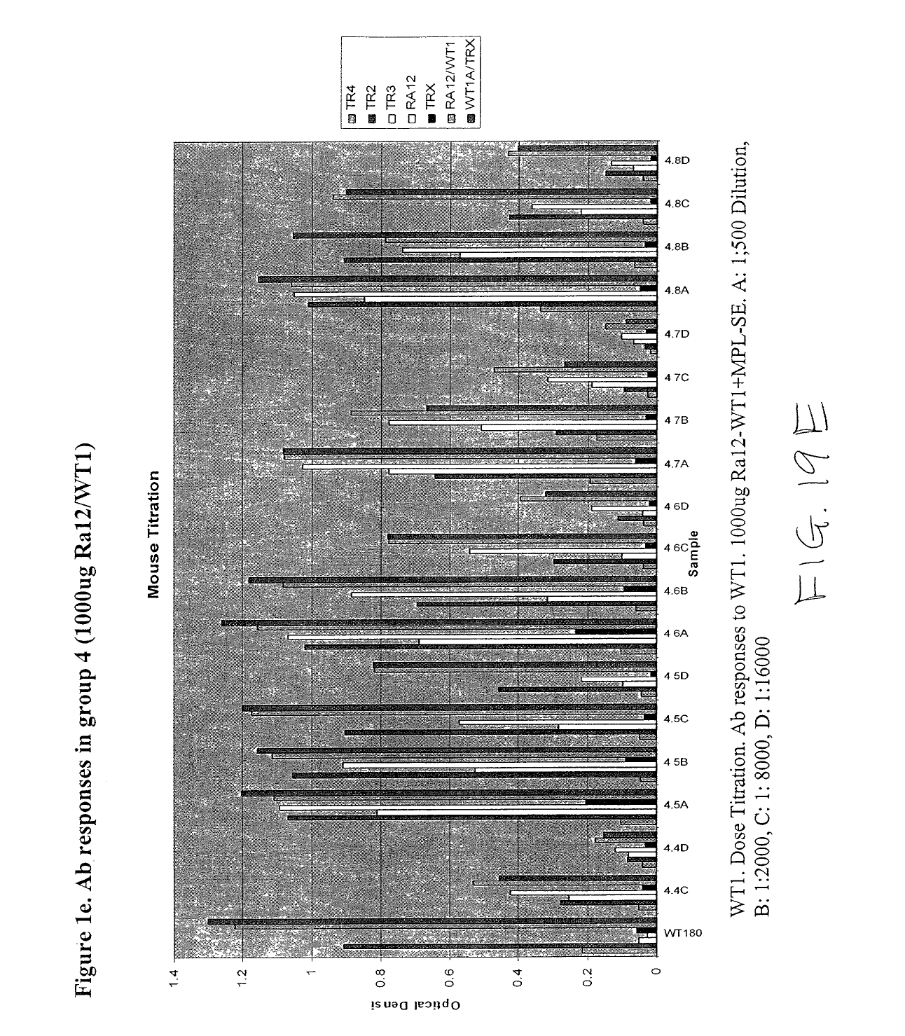

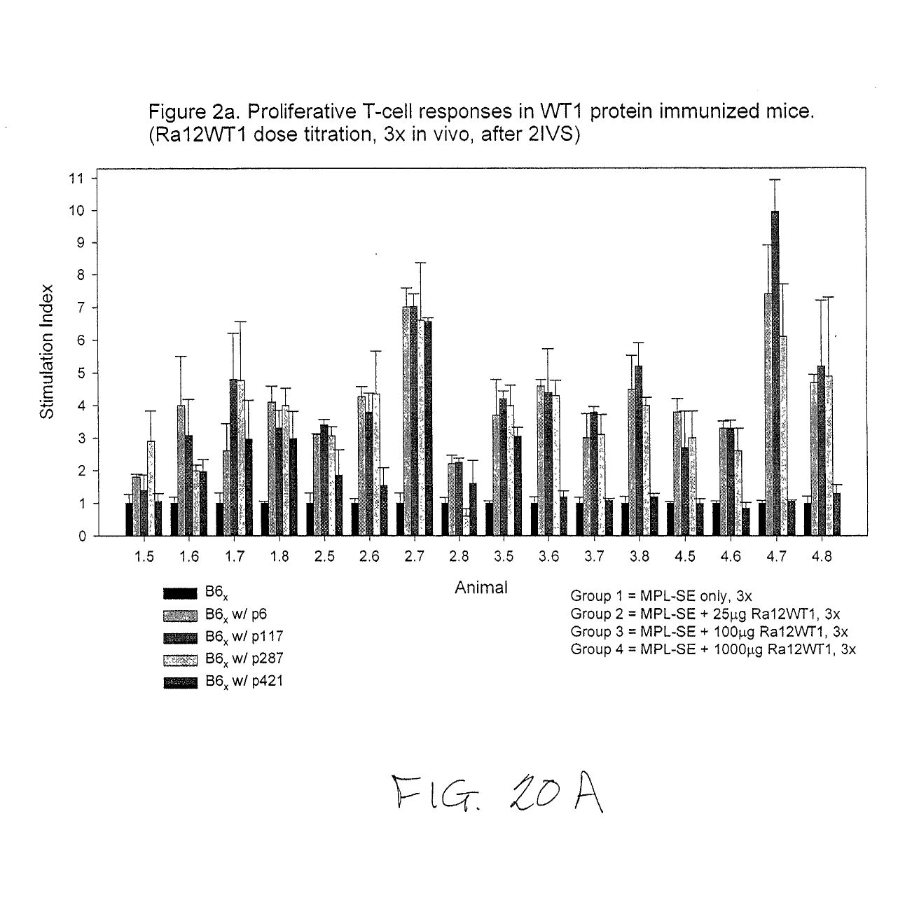

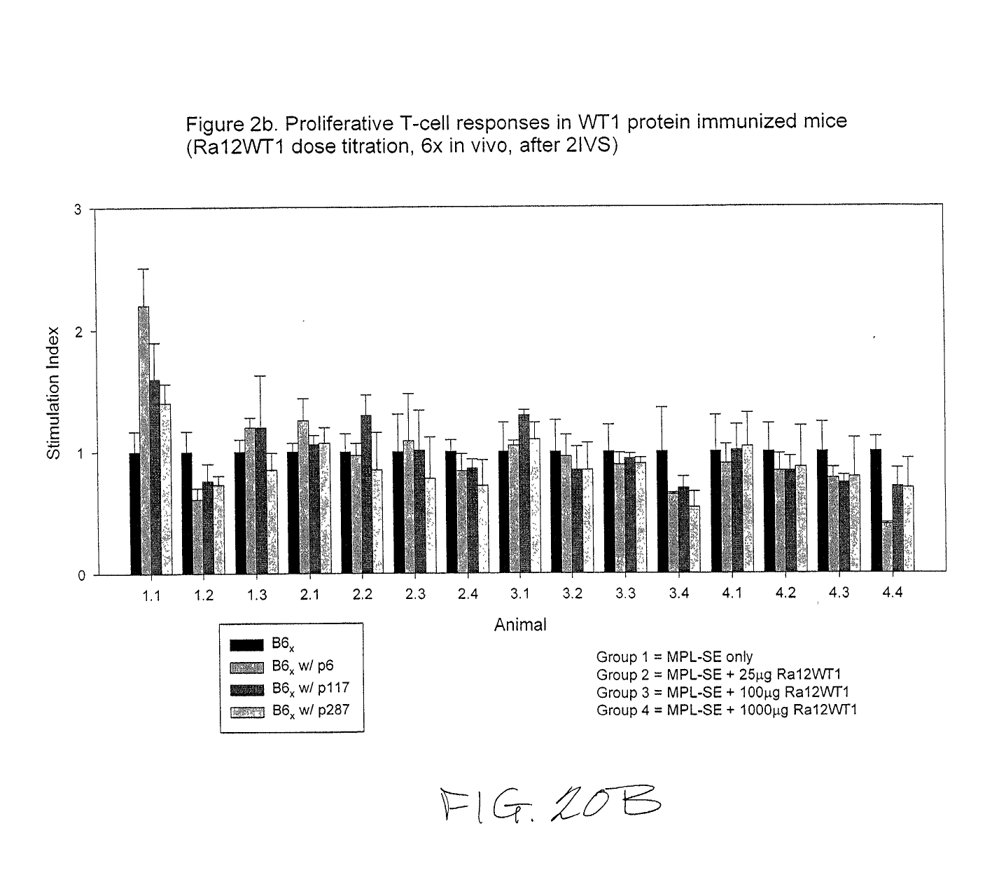

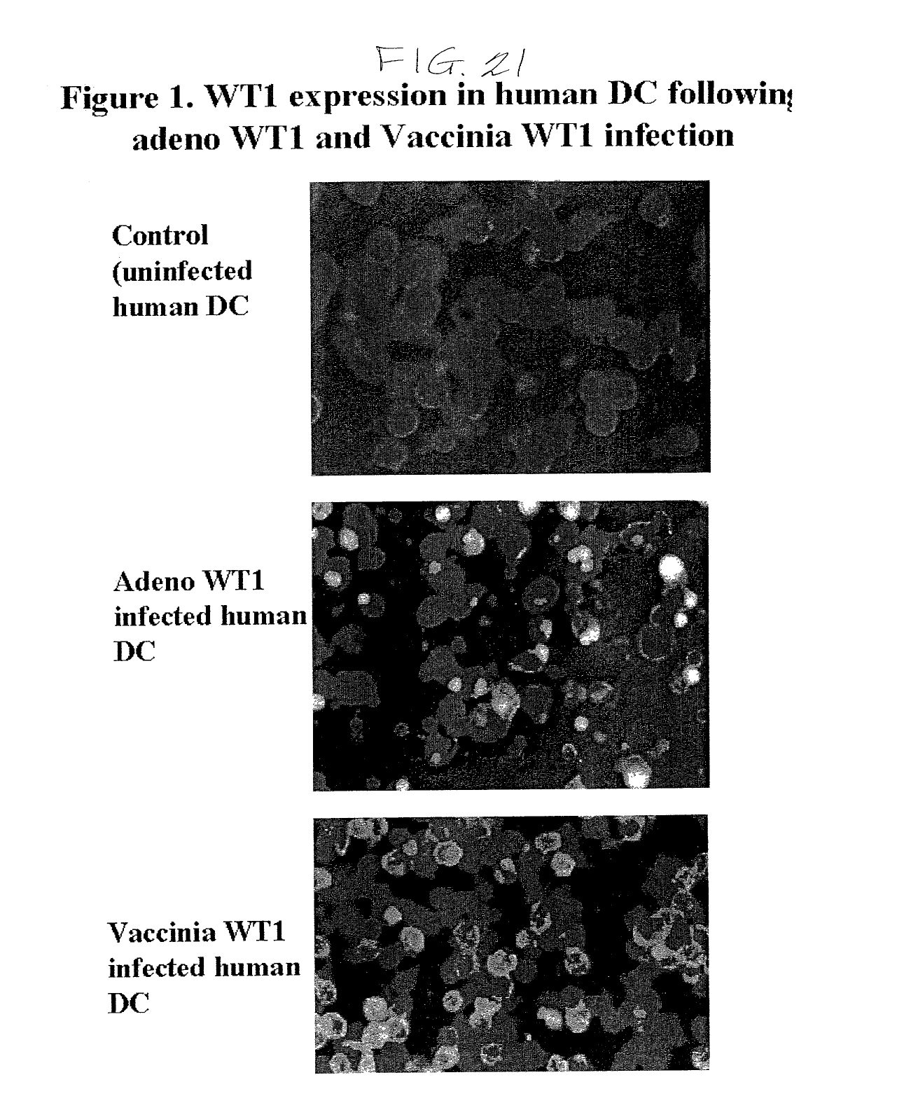

Vaccination to WT1 protein using MPL-SE as adjuvant, in a multiple dose titration study (doses ranging from 25 μg, 100 μg to 1000 μg WT1 protein) in female C57/B6 mice elicited a strong WT1-specific antibody response (FIG. 19) and cellular T-cell responses (FIG. 20).

No systemic histopathological or toxicological effects of immunization with WT1 protein was observed. No histological evidence for toxicity was seen in the following tissues: adrenal gland, brain, cecum, colon, duodenum, eye, femur and marrow, gall bladder, heart, ileum, jejunum, kidney, larynx, lacrimal gland, liver, lung, lymph node, muscle, esophagus, ovary, pancreas, parathyroid, salivary gland, sternum and marrow, spleen, stomach, thymus, trachea, thyroid, urinary bladder and uterus.

Special emphasis was put on evaluation of potential hematopoietic toxicity. The myeloid/erythroid ratio in sternum and femur marrow was normal. All evaluable blood cell counts and blood chemistry (BUN, creatinine, bilirubin, albumin, globulin) were within the normal range (Table LI).

Given that existent immunity to WT1 is present in some patients with leukemia and that vaccination to WT1 protein can elicit WT1 specific Ab and cellular T-cell responses in mice without toxicity to normal tissues, these experiments validate WT1 as a tumor/leukemia vaccine.

TABLE LI
WT1 Dose Titration Study
Clinical Chemistry and Hematology Analysis

| | K/uL | M/uL | g/dl | % | fL | pg | % | |
|---|---|---|---|---|---|---|---|---|
| Animal # | WBC | RBC | Hg. | HCT | MCV | MCH | MCHC | |
| Normal | 5.4-16.0 | 6.7-12.5 | 10.2-16.6 | 32-54 | 31-62 | 9.2-20.8 | 22.0-35.5 | |
| Group 1 | | | | | | | | |
| 1 (0) | 5.6 | 8.41 | 12.8 | 43.5 | 53 | 15.2 | 29.4 | |
| 2 (0) | 5.5 | 9.12 | 13.4 | 47.5 | 53 | 14.7 | 28.2 | |
| 3 (0) | 7.5 | 9.22 | 13.5 | 48 | 54 | 14.7 | 28.1 | |
| 4 (0) | 3.9 | 9.27 | 13.6 | 46 | 52 | 14.7 | 29.6 | |
| Mean | 5.6 | 9.0 | 13.3 | 46.3 | 53.0 | 14.8 | 28.8 | |
| STD | 1.5 | 0.4 | 0.4 | 2.0 | 0.8 | 0.3 | 0.8 | |
| Group 2 | | | | | | | | |
| 5 (1.5) | 6.6 | 9 | 13.1 | 46 | 54 | 14.5 | 28.5 | |
| 6 (1.6) | 5.2 | 8.58 | 12.6 | 44 | 53 | 14.7 | 28.6 | |
| 7 (1.7) | 7.8 | 9.21 | 13.6 | 46 | 53 | 14.7 | 29.6 | |
| 8 (1.8) | 6.3 | NA | NA | 41 | NA | NA | NA | |
| Mean | 6.5 | 8.9 | 13.1 | 44.3 | 53.3 | 14.6 | 28.9 | |
| STD | 1.1 | 0.3 | 0.5 | 2.4 | 0.6 | 0.1 | 0.6 | |
| Group 3 | | | | | | | | |
| 9 (2.5) | 8.3 | 9.16 | 13.6 | 50.3 | 55 | 14.9 | 27.1 | |
| 10 (2.6) | 5 | 8.78 | 13 | 44.2 | 50 | 14.8 | 29.3 | |
| 11 (2.7) | 4 | 8.94 | 13.2 | 48.3 | 54 | 14.7 | 27.3 | |
| 12 (2.8) | 8.2 | NA | NA | 41 | NA | NA | NA | |
| Mean | 6.4 | 9.0 | 13.3 | 46.0 | 53.0 | 14.8 | 27.9 | |
| STD | 2.2 | 0.2 | 0.3 | 4.2 | 2.6 | 0.1 | 1.2 | |
| Group 4 | | | | | | | | |
| 13 (3.5) | 6.1 | 8.82 | 13.1 | 46 | 54 | 14.9 | 28.5 | |
| 14 (3.6) | 6.1 | 8.64 | 12.9 | 46 | 54 | 15 | 28 | |
| 15 (3.7) | 9.3 | 8.93 | 13.2 | 48 | 55 | 14.8 | 27.5 | |
| 16 (3.8) | 4.8 | 8.19 | 12.6 | 44 | 55 | 15.3 | 28.6 | |
| Mean | 6.6 | 8.6 | 13.0 | 46.0 | 54.5 | 15.0 | 28.2 | |
| STD | 1.9 | 0.3 | 0.3 | 1.6 | 0.6 | 0.2 | 0.5 | |
| Group 5 | | | | | | | | |
| 17 (4.5) | 3.1 | 8.48 | 12.6 | 46 | 54 | 14.9 | 27.5 | |
| 18 (4.6) | 5.7 | 9.12 | 13.7 | 48 | 54 | 15 | 28.5 | |
| 19 (4.7) | 5.3 | 8.58 | 13 | 44.5 | 55 | 15.2 | 29.2 | |
| 20 (4.8) | 5.3 | NA | NA | 40 | NA | NA | NA | |
| Mean | 4.9 | 8.7 | 13.1 | 44.6 | 54.3 | 15.0 | 28.4 | |
| STD | 1.2 | 0.3 | 0.6 | 3.4 | 0.6 | 0.2 | 0.9 | |
| Group 6 | | | | | | | | |
| 21 (1.1) | 3.5 | 9.36 | 13.5 | 37.6 | 40 | 14.4 | 35.9 | |
| 22 (1.2) | 6.9 | 8.93 | 13.6 | 37.3 | 42 | 15.3 | 36.6 | |
| 23 (1.3) | 3.6 | 8.3 | 12.5 | 35.3 | 43 | 15.1 | 35.5 | |
| 24 (1.4) | NA | NA | NA | NA | NA | NA | NA | |
| Mean | 4.7 | 8.9 | 13.2 | 36.7 | 41.7 | 14.9 | 36.0 | |
| STD | 1.9 | 0.5 | 0.6 | 1.3 | 1.5 | 0.5 | 0.6 | |
| Group 7 | | | | | | | | |
| 25 (2.1) | 4 | NA | NA | 40 | NA | NA | NA | |
| 26 (2.2) | 7.4 | 9.12 | 13.2 | 38.5 | 42 | 14.5 | 34.3 | |
| 27 (2.3) | 4.5 | 8.19 | 12.1 | 34.5 | 42 | 14.8 | 35.1 | |
| 28 (2.4) | 5.8 | 8.25 | 12.3 | 34.1 | 41 | 14.9 | 36.1 | |
| Mean | 5.4 | 8.5 | 12.5 | 36.8 | 41.7 | 14.7 | 35.2 | |
| STD | 1.5 | 0.5 | 0.6 | 2.9 | 0.6 | 0.2 | 0.9 | |
| Group 8 | | | | | | | | |
| 29 (3.1) | 5.1 | 8.53 | 12.6 | 34.9 | 41 | 14.7 | 36 | |
| 30 (3.2) | 7.6 | 8.42 | 13 | 36.1 | 43 | 15.4 | 35.9 | |
| 31 (3.3) | 3.4 | 8.45 | 12.6 | 34.9 | 41 | 14.9 | 36.1 | |
| 32 (3.4) | 6.1 | 8.11 | 12.3 | 34.8 | 43 | 15.2 | 35.5 | |
| Mean | 5.6 | 8.4 | 12.6 | 35.2 | 42.0 | 15.1 | 35.9 | |
| STD | 1.8 | 0.2 | 0.3 | 0.6 | 1.2 | 0.3 | 0.3 | |
| Group 9 | | | | | | | | |
| 33 (4.1) | NA | NA | NA | NA | NA | NA | NA | |
| 34 (4.2) | 4.5 | 8.63 | 12.8 | 36.2 | 42 | 14.8 | 35.2 | |
| 35 (4.3) | 3.9 | 8.85 | 13 | 36.6 | 41 | 14.7 | 35.6 | |
| 36 (4.4) | 4.7 | 8.14 | 12.3 | 33.8 | 42 | 15.1 | 36.3 | |
| Mean | 4.4 | 8.5 | 12.7 | 35.5 | 41.7 | 14.9 | 35.7 | |
| STD | 0.4 | 0.4 | 0.4 | 1.5 | 0.6 | 0.2 | 0.6 | |
| | yes/no | K/uL | Abs. | Abs. | Abs. | Abs. | Abs. | Abs. |
| Animal # | Pit. clump | Platelets | Baso | Eos | Bands | Polys | Lymph | Mono |
| Normal | no | 150-1500 | 0.0-0.15 | 0.0-0.51 | 0.0-0.32 | 8.0-42.9 | 8.0-18.0 | 0.0-1.5 |
| Group 1 | | | K/uL | K/uL | K/uL | K/uL | K/uL | K/uL |
| 1 (0) | yes | 726 | 0 | 56 | 0 | 336 | 5208 | 0 |
| 2 (0) | no | 860 | 0 | 0 | 0 | 55 | 5445 | 0 |
| 3 (0) | no | 875 | 0 | 375 | 0 | 525 | 6525 | 75 |
| 4 (0) | yes | 902 | 0 | 0 | 0 | 156 | 3744 | 0 |
| Mean | | 840.8 | 0.0 | 107.8 | 0.0 | 268.0 | 5230.5 | 18.8 |
| STD | | 78.4 | 0.0 | 180.1 | 0.0 | 207.0 | 1144.8 | 37.5 |
| Group 2 | | | | | | | | |
| 5 (1.5) | no | 1193 | 0 | 132 | 0 | 792 | 5214 | 462 |
| 6 (1.6) | no | 1166 | 0 | 52 | 0 | 624 | 4472 | 52 |
| 7 (1.7) | no | 1087 | 0 | 234 | 0 | 1170 | 6396 | 0 |
| 8 (1.8) | yes | NA | 0 | 126 | 0 | 126 | 5922 | 126 |
| Mean | | 1148.7 | 0.0 | 136.0 | 0.0 | 678.0 | 5501.0 | 160.0 |
| STD | | 55.1 | 0.0 | 74.8 | 0.0 | 433.1 | 840.5 | 207.9 |
| Group 3 | | | | | | | | |
| 9 (2.5) | no | 705 | 0 | 166 | 0 | 664 | 7387 | 83 |
| 10 (2.6) | no | 1140 | 0 | 150 | 0 | 500 | 4350 | 0 |
| 11 (2.7) | no | 952 | 0 | 120 | 0 | 680 | 3200 | 0 |
| 12 (2.8) | yes | NA | 0 | 164 | 0 | 656 | 7216 | 164 |
| Mean | | 932.3 | 0.0 | 150.0 | 0.0 | 625.0 | 5538.3 | 61.8 |
| STD | | 218.2 | 0.0 | 21.2 | 0.0 | 83.9 | 2090.6 | 78.6 |
| Group 4 | | | | | | | | |
| 13 (3.5) | no | 785 | 0 | 488 | 0 | 732 | 4636 | 244 |
| 14 (3.6) | yes | 973 | 0 | 0 | 0 | 488 | 5307 | 305 |
| 15 (3.7) | yes | 939 | 0 | 465 | 0 | 558 | 7812 | 465 |
| 16 (3.8) | yes | 1622 | 0 | 192 | 0 | 480 | 4080 | 48 |
| Mean | | 1079.8 | 0.0 | 286.3 | 0.0 | 564.5 | 5458.8 | 265.5 |
| STD | | 370.6 | 0.0 | 233.4 | 0.0 | 117.0 | 1647.1 | 172.4 |
| Group 5 | | | | | | | | |
| 17 (4.5) | no | 892 | 0 | 31 | 0 | 620 2449 | 0 | |
| 18 (4.6) | yes | 966 | 57 | 114 | 0 | 855 | 4674 | 0 |
| 19 (4.7) | yes | 883 | 0 | 53 | 0 | 742 | 4452 | 53 |
| 20 (4.8) | yes | NA | 0 | 106 | 0 | 53 | 5141 | 0 |
| Mean | | 913.7 | 14.3 | 76.0 | 0.0 | 567.5 | 4179.0 | 13.3 |
| STD | | 45.5 | 28.5 | 40.4 | 0.0 | 356.2 | 1188.5 | 26.5 |
| Group 6 | | | | | | | | |
| 21 (1.1) | yes | 784 | 0 | 35 | 0 | 385 | 2870 | 210 |
| 22 (1.2) | yes | 806 | 0 | 69 | 0 | 207 | 6486 | 138 |
| 23 (1.3) | yes | 790 | 0 | 180 | 0 | 396 | 2988 | 36 |
| 24 (1.4) | NA | NA | NA | NA | NA | NA | NA | NA |
| Mean | | 793.3 | 0.0 | 94.7 | 0.0 | 329.3 | 4114.7 | 128.0 |
| STD | | 11.4 | 0.0 | 75.8 | 0.0 | 106.1 | 2054.5 | 87.4 |
| Group 7 | | | | | | | | |
| 25 (2.1) | yes | NA | 0 | 80 | 0 | 200 | 3720 | 0 |
| 26 (2.2) | yes | 753 | 0 | 0 | 0 | 518 | 6734 | 148 |
| 27 (2.3) | yes | 725 | 0 | 90 | 0 | 225 | 4140 | 45 |
| 28 (2.4) | yes | 792 | 0 | 232 | 0 | 754 | 4814 | 0 |
| Mean | | 756.7 | 0.0 | 100.5 | 0.0 | 424.3 | 4852.0 | 48.3 |
| STD | | 33.7 | 0.0 | 96.5 | 0.0 | 263.0 | 1333.1 | 69.8 |
| Group 8 | | | | | | | | |
| 29 (3.1) | yes | 784 | 0 | 153 | 0 | 561 | 4233 | 153 |
| 30 (3.2) | yes | 512 | 0 | 152 | 0 | 304 | 6992 | 152 |
| 31 (3.3) | yes | 701 | 0 | 0 | 0 | 238 | 3094 | 68 |
| 32 (3.4) | yes | 631 | 0 | 305 | 0 | 305 | 5368 | 122 |
| Mean | | 657.0 | 0.0 | 152.5 | 0.0 | 352.0 | 4921.8 | 123.8 |
| STD | | 115.1 | 0.0 | 124.5 | 0.0 | 142.8 | 1663.3 | 39.9 |
| Group 9 | | | | | | | | |
| 33 (4.1) | NA | NA | NA | NA | NA | NA | NA | NA |
| 34 (4.2) | yes | 724 | 0 | 125 | 0 | 540 | 3780 | 45 |
| 35 (4.3) | yes | 758 | 0 | 117 | 0 | 429 | 3315 | 39 |
| 36 (4.4) | yes | 808 | 0 | 47 | 0 | 329 | 4089 | 235 |
| Mean | | 763.3 | 0.0 | 96.3 | 0.0 | 432.7 | 3728.0 | 106.3 |
| STD | | 42.3 | 0.0 | 42.9 | 0.0 | 105.5 | 389.6 | 111.5 |
| | mg/dl | mg/dl | g/dl | g/dl | g/dl | mg/dl | | |
| Animal # | BUN | Creatinine | T. protein | Albumin | Globulin | T. Bilirubin | | |
| Normal | 13.9-28.3 | 0.3-1.0 | 4.0-8.6 | 2.5-4.8 | 1.5-3.8 | 0.10-0.90 | | |
| Group 1 | | | | | | | | |
| 1 (0) | NA | NA | NA | NA | NA | NA | | |
| 2 (0) | 28 | 0.5 | 4.9 | 3.7 | 1.2 | 0.3 | | |
| 3 (0) | 25 | 0.5 | 4.9 | 3.8 | 1.1 | 0.2 | | |
| 4 (0) | 27 | 0.5 | 4.7 | 3.7 | 1 | 0.2 | | |
| Mean | 26.7 | 0.5 | 4.8 | 3.7 | 1.1 | 0.2 | | |
| STD | 1.5 | 0.0 | 0.1 | 0.1 | 0.1 | 0.1 | | |
| Group 2 | | | | | | | | |
| 5 (1.5) | 34 | 0.5 | 4.6 | 3.6 | 1 | 0.2 | | |
| 6 (1.6) | 31 | 0.4 | 4.6 | 3.3 | 1.3 | 0.2 | | |
| 7 (1.7) | 34 | 0.6 | 4.9 | 4 | 0.9 | 0.3 | | |
| 8 (1.8) | NA | NA | NA | NA | NA | NA | | |
| Mean | 33.0 | 0.5 | 4.7 | 3.6 | 1.1 | 0.2 | | |
| STD | 1.7 | 0.1 | 0.2 | 0.4 | 0.2 | 0.1 | | |
| Group 3 | | | | | | | | |
| 9 (2.5) | NA | NA | NA | NA | NA | NA | | |
| 10 (2.6) | 33 | 0.5 | 4.6 | 3.6 | 1 | 0.3 | | |
| 11 (2.7) | NA | NA | NA | NA | NA | NA | | |
| 12 (2.8) | 31 | 0.5 | 4.8 | 3.7 | 1.1 | 0.2 | | |
| Mean | 32.0 | 0.5 | 4.7 | 3.7 | 1.1 | 0.3 | | |
| STD | 1.4 | 0.0 | 0.1 | 0.1 | 0.1 | 0.1 | | |
| Group 4 | | | | | | | | |
| 13 (3.5) | 32 | 0.7 | 4.6 | 3.4 | 1.2 | 0.2 | | |
| 14 (3.6) | 34 | 0.4 | 4.8 | 3.8 | 1 | 0.2 | | |
| 15 (3.7) | 30 | 0.4 | 4.7 | 3.4 | 1.3 | 0.2 | | |
| 16 (3.8) | 24 | 0.3 | 5.1 | 3.8 | 1.3 | 0.2 | | |
| Mean | 30.0 | 0.5 | 4.8 | 3.6 | 1.2 | 0.2 | | |
| STD | 4.3 | 0.2 | 0.2 | 0.2 | 0.1 | 0.0 | | |
| Group 5 | | | | | | | | |
| 17 (4.5) | 22 | 0.4 | 4.6 | 3.3 | 1.3 | 0.2 | | |
| 18 (4.6) | 31 | 0.5 | 4.9 | 3.7 | 1.2 | 0.2 | | |
| 19 (4.7) | 23 | 0.6 | 4.8 | 3.6 | 1.2 | 0.2 | | |
| 20 (4.8) | 28 | 0.5 | 4.5 | 3.4 | 1.1 | 0.2 | | |
| Mean | 26.0 | 0.5 | 4.7 | 3.5 | 1.2 | 0.2 | | |
| STD | 4.2 | 0.1 | 0.2 | 0.2 | 0.1 | 0.0 | | |
| Group 6 | | | | | | | | |
| 21 (1.1) | 28 | 0.3 | 5.1 | 3.4 | 1.7 | 0.2 | | |
| 22 (1.2) | 36 | 0.3 | 5.1 | 3.8 | 1.3 | 0.2 | | |
| 23 (1.3) | 32 | 0.4 | 4.9 | 3.5 | 1.4 | 0.1 | | |
| 24 (1.4) | NA | NA | NA | NA | NA | NA | | |
| Mean | 32.0 | 0.3 | 5.0 | 3.6 | 1.5 | 0.2 | | |
| STD | 4.0 | 0.1 | 0.1 | 0.2 | 0.2 | 0.1 | | |
| Group 7 | | | | | | | | |
| 25 (2.1) | 32 | 0.2 | 5 | 3.4 | 1.6 | 0.2 | | |
| 26 (2.2) | 24 | 0.3 | 4.2 | 2.8 | 1.4 | 0.1 | | |
| 27 (2.3) | 28 | 0.3 | 4.8 | 3.2 | 1.6 | 0.2 | | |
| 28 (2.4) | 27 | 0.3 | 5 | 3.4 | 1.6 | 0.1 | | |
| Mean | 27.8 | 0.3 | 4.8 | 3.2 | 1.6 | 0.2 | | |
| STD | 3.3 | 0.0 | 0.4 | 0.3 | 0.1 | 0.1 | | |
| Group 8 | | | | | | | | |
| 29 (3.1) | 32 | 0.3 | 4.9 | 3.3 | 1.6 | 0.2 | | |
| 30 (3.2) | NA | NA | NA | NA | NA | NA | | |
| 31 (3.3) | 18 | 0.3 | 4.8 | 3.1 | 1.7 | 0.2 | | |
| 32 (3.4) | 26 | 0.2 | 4.2 | 2.9 | 1.3 | 0 | | |
| Mean | 25.3 | 0.3 | 4.6 | 3.1 | 1.5 | 0.1 | | |
| STD | 7.0 | 0.1 | 0.4 | 0.2 | 0.2 | 0.1 | | |
| Group 9 | | | | | | | | |
| 33 (4.1) | 25 | 0.2 | 4.1 | 2.7 | 1.4 | 0.3 | | |
| 34 (4.2) | NA | NA | NA | NA | NA | NA | | |
| 35 (4.3) | 23 | 0.2 | 4.7 | 3.1 | 1.6 | 0.2 | | |
| 36 (4.4) | 29 | 0.3 | 4.7 | 3.2 | 1.5 | 0.3 | | |
| Mean | 25.7 | 0.2 | 4.5 | 3.0 | 1.5 | 0.3 | | |
| STD | 3.1 | 0.1 | 0.3 | 0.3 | 0.1 | 0.1 | | |

Abbreviations: WBC: white blood cells; RBC: red blood cells; Hg.: hemoglobin; HCT: hematocrit; MCV: Mean corpuscular volume; MCH: mean corpuscular hemoglobin; MCHC: mean corpuscular hemoglobin concentration; Plt.: platelets; Abs.: Absolute; Baso: basophils; Eos: eosinophils; Abs. Bands: immature neutrophils; Polys: polymorphonuclear cells; Lymph: lymphocytes; Mono: monocytes; BUN: blood urea nitrogen

### Example 14

### Elicitation of Human WT1-Specific T-Cell Responses by Whole Gene In Vitro Priming

This example demonstrates that WT1 specific T-cell responses can be generated from the blood of normal individuals.

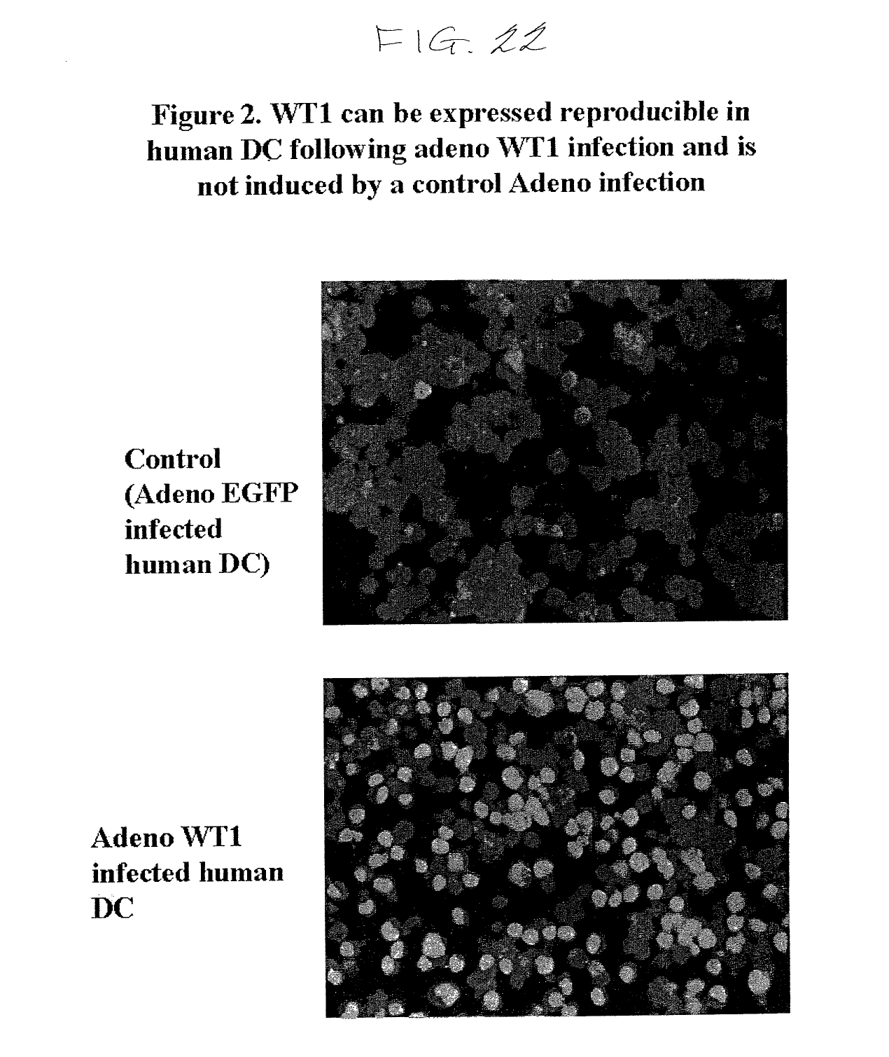

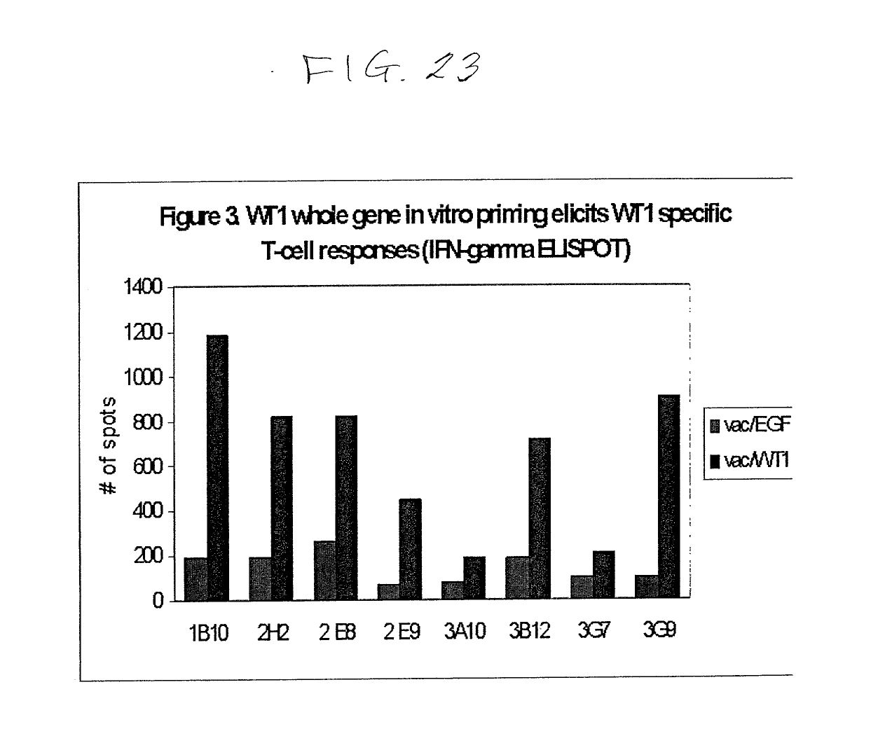

Dendritic cells (DC) were differentiated from monocyte cultures derived from PBMC of normal donors by growth for 4-10 days in RPMI medium containing 10% human serum, 50 ng/ml GMCSF and 30 ng/ml IL-4. Following culture, DC were infected 16 hours with recombinant WT1-expressing vaccinia virus at an M.O.I. of 5, or for 3 days with recombinant WT1-expressing adenovirus at an M.O.I. of 10 (FIGS. 21 and 22). Vaccinia virus was inactivated by U.V. irradiation. CD8+ T-cells were isolated by positive selection using magnetic beads, and priming cultures were initiated in 96-well plates. Cultures were restimulated every 7-10 days using autologous dendritic cells adeno or vaccinia infected to express WT1. Following 3-6 stimulation cycles, CD8+ lines could be identified that specifically produced interferon-gamma when stimulated with autologous-WT1-expressing dendritic cells or fibroblasts. The WT1-specific activity of these lines could be maintained following additional stimulation cycles. These lines were demonstrated to specifically recognize adeno or vaccinia WT1 infected autologous dendritic cells but not adeno or vaccinia EGFP-infected autologous dendritic cells by Elispot assays (FIG. 23).

### Example 15

### Formulation of Ra12-WT1 for Injection: Use of Excipients to Stabilize Lyophilized Product

This example describes the formulation that allows the complete solubilization of lyophilized Ra12-WT1.

The following formulation allowed for the recombinant protein Ra12-WT1 to be dissolved into an aqueous medium after being lyophylized to dryness:

Recombinant Ra12-WT1 concentration: 0.5-1.0 mg/ml; Buffer: 10-20 mM Ethanolamine, pH 10.0; 1.0-5.0 mM Cysteine; 0.05 % Tween-80 (Polysorbate-80); Sugar: 10% Trehalose (T5251, Sigma, MO) 10% Maltose (M9171, Sigma, MO) 10% Sucrose (S7903, Sigma, MO) 10% Fructose (F2543, Sigma, MO) 10% Glucose (G7528, Sigma, MO).

The lyophilized protein with the sugar excipient was found to dissolve significantly more than without the sugar excipient. Analysis by coomassie stained SDS-PAGE showed no signs of remaining solids in the dissolved material.

### Example 16

### Formulation of a WT1 Protein Vaccine

This example describes the induction of WT1-specific immune responses following immunization with WT1 protein and 2 different adjuvant formulations.

According to this example, WT1 protein in combination with MPL-SE induces a strong Ab and Interferon-γ (IFN-γ) response to WT1. Described in detail below are the methods used to induce WT1 specific immune responses following WT1 protein immunization using MPL-SE or Enhanzyn as adjuvant in C57/B6 mice.

C57BL/6 mice were immunized with 20 μg rRa12-WT1 combined with either MPL-SE or Enhanzyn adjuvants. One group of control mice was immunized with rRa12-WT1 without adjuvant and one group was immunized with saline alone. Three intramuscular (IM) immunizations were given, three weeks apart. Spleens and sera were harvested 2 weeks post-final immunization. Sera were analyzed for antibody responses by ELISA on plates coated with Ra12-WT1 fusion, Ra12 or WT1TRX. Similar levels of IgG2a and IgG1 antibody titers were observed in mice immunized with Ra12-WT1+MPL-SE and Ra12-WT1+Enhanzyn. Mice immunized with rRa12-WT1 without adjuvant showed lower levels of IgG2a antibodies.

CD4 responses were assessed by measuring Interferon-γ production following stimulation of splenocytes in vitro with rRa12-WT1, rRa12 or with WT1 peptides p6, p117 and p287. Both adjuvants improved the CD4 responses over mice immunized with rRA12-WT1 alone. Additionally, the results indicate that rRA12-WT1+MPL-SE induced a stronger CD4 response than did rRA12-WT1+Enhanzyn. IFN-γ OD readings ranged from 1.4-1.6 in the mice immunized with rRA12-WT1+MPL-SE as compared to 1-1.2 in the mice immunized with rRA12-WT1+Enhanzyn. Peptide responses were only observed against p117, and then only in mice immunized with rRa12-WT1+MPL-SE. Strong IFN-γ responses to the positive control, ConA, were observed in all mice. Only responses to ConA were observed in the negative control mice immunized with saline indicating that the responses were specific to rRA12-WT1.

### Example 17

### Construction of a Randomly Mutated WT1 Library

The nucleic acid sequence of human WT1 was randomly mutated using a polymerase chain reaction method in the presence of 8-oxo dGTP and dPTP journal of Molecular Biology 1996; 255:589-603). The complete unspliced human WT1 gene is disclosed in SEQ ID NO:380 and the corresponding protein sequence is set forth in SEQ ID NO:404. A splice variant of WT1 was used as a template for the PCR reactions and is disclosed in SEQ ID NOs:381 (DNA) and 408 (protein). Conditions were selected so that the frequency of nucleic acid alterations led to a targeted change in the amino acid sequence, usually 5-30% of the PCR product. The mutated PCR product was then amplified in the absence of the nucleotide analogues using the four normal dNTPs. This PCR product was subcloned into mammalian expression vectors and viral vectors for immunization. This library, therefore, contains a mixed population of randomly mutated WT1 clones. Several clones were selected and sequenced. The mutated WT1 variant DNA sequences are disclosed in SEQ ID NOs:377-379 and the predicted amino acid sequences of the variants are set forth in SEQ ID NOs:405-407. These altered sequences, and others from the library, can be used as immunogens to induce stronger T cell responses against WT1 protein in cancer cells.

### Example 18

### Construction of WT1-Lamp Fusions

A tripartite fusion was constructed using the polymerase chain reaction and synthetic oligonucleotides containing the desired junctions of human lysosomal associated membrane protein-1 (LAMP-1) and a splice variant of the human WT1 sequence. The splice variant of WT1 and the LAMP-1 sequence used for these fusions are disclosed in SEQ ID NOs:381 and 383. Specifically, the signal peptide of LAMP-1 (base pairs 1-87 of LAMP) was fused to the 5-prime end of the human WT1 open reading frame (1,290 base pairs in length), then the transmembrane and cytoplasmic domain of LAMP-1 (base pairs 1161 to 1281 of LAMP) was fused to the 3-prime end of the WT1 sequence. The sequence of the resulting WT1-LAMP construct is set forth in SEQ ID NO:382 (DNA) and SEQ ID NO:409 (protein). The construct was designed so that when it is expressed in eukaryotic cells, the signal peptide directs the protein to the endoplasmic reticulum (ER) where the localization signals in the transmembrane and cytoplasmic domain of LAMP-1 direct transport of the fusion protein to the lysosomal location where peptides are loaded on to Class II MHC molecules.

### Example 19

### Construction of WT1-Ubiquitin Fusions for Enhanced MHC Class I Presentation

The human ubiquitin open reading frame (SEQ ID NO:384) was mutated such that the nucleotides encoding the last amino acid encode an alanine instead of a glycine. This mutated open reading frame was cloned in frame just upstream of the first codon of a splice variant of human WT1 (SEQ ID NOs:381 and 408, DNA and protein, respectively). The G→A mutation prevents co-translational cleavage of the nacent protein by the proteases that normally process poly-ubiquitin during translation. The DNA and predicted amino acid sequence for the resulting contruct are set forth in SEQ ID NOs:385 and 410, respectively. The resulting protein demonstrated decreased cellular cytotoxicity when it was expressed in human cells. Whereas it was not possible to generate stable lines expressing native WT1, cell lines expressing the fusion protein were readily obtained. The resulting protein is predicted to be targeted to the proteosome by virtue of the added ubiquitin molecule. This should result in more efficient recognition of the protein by WT1 specific CD8+ T cells.

### Example 20

### Construction of an Adenovirus Vector Expressing Human WT1

A splice variant of human WT1 (SEQ ID NO:381) was cloned into an E1 and E3 deleted adenovirus serotype 5 vector. The expression of the WT1 gene is controlled by the CMV promoter mediating high levels of WT1 protein expression. Infection of human cells with this reagent leads to a high level of expression of the WT1 protein. The antigenic nature of the adenoviral proteins introduced into the host cell during and produced at low levels subsequent to infection can act to increase immune surveillance and immune recognition of WT1 as an immunological target. This vector can be also used to generate immune responses against the WT1 protein when innoculated into human subjects. If these subjects are positive for WT1 expressing tumor cells the immune response could have a theraputic or curative effect on the course of the disease.

### Example 21

### Construction of a Vaccinia Virus Vector Expressing Human WT1

A splice variant of the full length human WT1 gene (SEQ ID NO:381) was cloned into the thymidine kinase locus of the Western Reserve strain of the vaccinia virus using the pSC11 shuttle vector. The WT1 gene is under the control of a hybrid vaccinia virus promoter that mediates gene expression throughout the course of vaccinia virus infection. This reagent can be used to express the WT1 protein in human cells in vivo or in vitro. WT1 is a self protein that is overexpressed on some human tumor cells. Thus, immunological responses to WT1 delivered as a protein are unlikely to lead to Major Histocompatibility Class I (MHC class I)-mediated recognition of WT1. However, expression of the protein in the intracellular compartment by the vaccinia virus vector will allow high level MHC class I presentation and recognition of the WT1 protein by CD8+ T cells. Expression of the WT1 protein by the vaccinia virus vector will also lead to presentation of WT1 peptides in the context of MHC class II and thus to recognition by CD4+ T cells.

The uses of this invention include its use as a cancer vaccine. Immunization of human subjects bearing WT1 positive tumors could lead to a theraputic or curative response. The expression of WT1 within the cell will lead to recognition of the protein by both CD4 and CD8 positive T cells.

### Example 22

### Generation of WT1-Specific CD8+ T-Cell Clones Using Whole Gene Priming

Dendritic cells (DC) were differentiated from monocyte cultures derived from PBMC of normal donors by growth for 4-6 days in RPMI medium containing 10% human serum, 50 ng/ml GM-CSF and 30 ng/ml IL-4. Following culture, DC were infected 16 hours with recombinant WT1-expressing vaccinia virus (described in Example 21) at a multiplicity of infection (MOI) of 5 or for 3 days with recombinat WT1-expressing adenovirus at an MOI of 10. Vaccinia virus was inactivated by U.V. irradiation. CD8+ T-cells were isolated by negative depletion using magnetic beads, and priming cultures were initiated in 96-well plates. Cultures were restimulated every 7-10 days using autologous dendritic cells infected with adeno or vaccinia virus engineered to express WT1. Following 4-5 stimulation cycles, CD8+ T-cell lines could be identified that specifically produced interferon-gamma when stimulated with autologous-WT1 expressing dendritic cells or fibroblasts. These lines were cloned and demonstrated to specifically recognize WT1 transduced autologous fibroblasts but not EGFP transduced fibroblasts by Elispot assays.

The Wilms' tumor (WT1) gene participates in leukemogenesis and is overexpressed in most human leukemias as well as in several solid tumors. Previous studies in humans have demonstrated the presence of WT1 specific antibody (Ab) responses in 16/63 (25%) of AML and in 15/81 (19%) of CML patients studied. Previous studies in mice have shown that WT1 peptide based vaccines elicit WT1 specific Ab, Th and CTL responses. The use of peptides as vaccines in humans is limited by their HLA restriction and the tendency to elicit peptide specific responses and only in a minority of patients tumor specific CTL. The advantages of whole gene immunization are that several helper and CTL epitopes can be included in a single vaccine, thus not restricting the vaccine to specific HLA types. The data disclosed herein demonstrate the induction of WT1 specific immune responses using whole gene in vitro priming. and that WT1 specific CD8+ T-cell clones can be generated. Given that existent immunity to WT1 is present in some patients with leukemia and that murine and human WT1 are 96% identical at the amino acid level and vaccination to WT1 protein, DNA or peptides can elicit WT1 specific Ab, and cellular T-cell responses in mice without toxicity to normal tissues in mice, these human in vitro priming experiments provide further validation of WT1 as a tumor/leukemia vaccine. Furthermore, the ability to generate WT1 specific CD8+ T-cell clones may lead to the treatment of malignancies associated with WT1 overexpression using genetically engineered T-cells.

### Example 23

### Recombinant Constructs for Clinical Manufacturing of WT1

Five constructs were made as described in detail below, for the production of clinical grade WT1.

Design of Ra12/WT-E (SEQ ID NOs:388 (cDNA) and 391 (protein)) and WT-1 E (SEQ ID NOs:386 (cDNA) and 395 (protein)) with No His Tag:

The WT-1 E reading frame was PCR amplified with the following primers for the non-His non fusion construct:

| | |
|---|---|
| PDM-780 (SEQ ID NO: 396) | 5′ gacgaaagcatatgcactccttcatcaaac 3′ Tm 60° C. |
| PDM-779 (SEQ ID NO: 397) | 5′ cgcgtgaattcatcactgaatgcctctgaag 3′ Tm 63° C. |

The following PCR cycling conditions were used: 10μl 10X Pfu buffer, 1μl 10 mM dNTPs, 2μl 10μM each oligo, 83μl sterile water 1.5μl Pfu DNA polymerase (Stratagene, La Jolla, Calif.), 50 ηg DNA (pPDMRa12 WT-1 No His). The reaction was denatured initially at 96° C. for 2 minutes, followed by 40 cylces of 96° C. for 20 seconds, 62° C. for 15 seconds, and 72° C. for 1 minute and 40 seconds. This was followed by a final extension of 72° C. for 4 minutes. The PCR product was digested with NdeI and EcoRI and cloned into pPDM His (a modified pET28 vector) that had been digested with NdeI and EcoRI. The construct was confirmed through sequence analysis and then transformed into BLR (DE3) pLys S and HMS 174 (DE3) pLys S cells. This construct—pPDM WT-1 E was then digested with NcoI and XbaI and used as the vector backbone for the NcoI and XbaI insert from pPDM Ra12 WT-1 F (see below). The construct was confirmed through sequence analysis and then tranformed into BLR (DE3) pLys S and HMS 174 (DE3) pLys S cells. Protein expression was confirmed by Coomassie stained SDS-PAGE and N-terminal protein sequence analysis.

Design of Ra12-WT-1-F (a.a. 1-281) with No His tTag (SEQ ID NOs:389 (cDNA) and 393 (protein)):

The Ra12 WT-1 reading frame was PCR amplified with the following primers:

| | |
|---|---|
| PDM-777 (SEQ ID NO:398) | 5′ cgataagcatatgacggccgcgtccgataac 3′ Tm 66° C. |
| PDM-779 (SEQ ID NO:399) | 5′ cgcgtgaattcatcactgaatgcctctgaag 3′ Tm 63° C. |

The following PCR cycling conditions were used: 10μl 10X Pfu buffer, 1 μl 10 mM dNTPs, 2 μl 10 μM each oligo, 83 μl sterile water 1.5 μl Pfu DNA polymerase (Stratagene, La Jolla, Calif.), 50 ηg DNA (pPDMRa12 WT-1 No His). The reaction was denatured initially at 96° C. for 2 minutes, followed by 40 cylces of 96° C. for 20 seconds, 58° C. for 15 seconds, and 72° C. for 3 minutes. This was followed by a final extension of 72° C. for 4 minutes. The PCR product was digested with NdeI and cloned into pPDM His that had been digested with NdeI and Eco72I. The sequence was confirmed through sequence analysis and then transformed into BLR (DE3) pLys S and HMS 174 (DE3) pLysS cells. Protein expression was confirmed by Coomassie stained SDS-PAGE and N-terminal protein sequence analysis.

Design of Ra12-WT-1 with No His Tag (SEQ ID NOs:390 (cDNA) and 392 (protein)):

The Ra12 WT-1 Reading Frame was PCR Amplified with the following Primers:

| | |
|---|---|
| PDM-777 (SEQ ID NO:400) | 5′ cgataagcatatgacggccgcgtccgataac 3′ Tm 66° C. |
| PDM-778 (SEQ ID NO:401) | 5′ gtctgcagcggccgctcaaagcgccagc 3′ Tm 70° C. |

The following PCR cycling conditions were used: 1 μl 10X Pfu buffer, 1 μl 10 mM dNTPs, 2 μl 10 μM each oligo, 83 μl sterile water 1.5 μl Pfu DNA polymerase (Stratagene, La Jolla, Calif.), 50 ηg DNA (pPDMRa12 WT-1 No His). The reaction was denatured initially at 96° C. for 2 minutes, followed by 40 cylces of 96° C. for 20 seconds, 68° C. for 15 seconds, and 72° C. for 2 minutes and 30 seconds. This was followed by a final extension of 72° C. for 4 minutes. The PCR product was digested with NotI and NdeI and cloned into pPDM His that had been digested with NdeI and NotI. The sequence was confirmed through sequence anaysis and then transformed into BLR (DE3) pLys S and HMS 174 (DE3) pLysS cells. Protein expression was confirmed by Coomassie stained SDS-PAGE and N-terminal protein sequence analysis.

Design of WT-1 C (a.a. 69-430) in E. coli without His Tag (SEQ ID NOs:387 (cDNA) and 394 (protein)):

The WT-1 C reading frame was PCR amplified with the following primers:

PDM-780 (SEQ ID NO:402) 5′ gacgaaagcatatgcactccttcatcaaac 3′ Tm 60° C

PDM-778 (SEQ ID NO:403) 5′ gtctgcagcggccgctcaaagcgccagc 3′ Tm 70° C.

The following PCR cycling conditions were used: 10 μl 10X Pfu buffer, 1 μl 10 mM dNTPs, 2 μl 10 μM each oligo, 83 μl sterile water 1.51 μl Pfu DNA polymerase (Stratagene, La Jolla, Calif.), 50 ηg DNA (pPDMRa12 WT-1 No His). The reaction was denatured initially at 96° C. for 2 minutes, followed by 40 cylces of 96° C. for 20 seconds, 62° C. for 15 seconds, and 72° C. for 2 minutes. This was followed by a final extension of 72° C. for 4 minutes. The PCR product was digested with NdeI and cloned into pPDM His that had been digested with NdeI and Eco72I. The sequence was confirmed through sequence analysis and then transformed into BLR (DE3) pLys S and HMS 174 (DE3) pLys S cells. Protein expression was confirmed by Coomassie stained SDS-PAGE and N-terminal protein sequence analysis.

### Example 24

### Generation of WT1 -Specific CD8+ T Cell Clones Using Whole Gene Priming and Identification of an HLA-A2-Restricted WT1 Epitope

In this example, Adeno and Vaccinia virus delivery vehicles were used to generate WT1-specific T cell lines. A T cell clone from the line was shown to be specific for WT1 and further, the epitope recognized by this clone was identified.

Dendritic cells (DC) were differentiated from monocyte cultures derived from PBMC of normal donors by growth for 4-6 days in RPMI medium containing 10% human serum, 50 ng/ml GM-CSF and 30 ng/ml IL-4. Following culture, DC were infected 16 hours with recombinant WT1-expressing vaccinia virus at a multiplicity of infection (MOI) of 5 or for 2-3 days with recombinant WT1 -expressing adeno virus at an MOI of 3-10. Vaccinia virus was inactivated by U.V. irradiation. CD8+ T-cells were isolated by negative depletion using antibodies to CD4, CD14, CD16, CD19 and CD56+ cells, followed by magnetic beads specific for the Fe portion of these Abs.

Priming cultures were initiated in 96-well plates. Cultures were restimulated every 7-14 days using autologous dendritic cells infected with adeno or vaccinia virus engineered to express WT1. Following 4-5 stimulation cycles, CD8+ T cell lines could be identified that specifically produced interferon-γ (IFN-γ) when stimulated with autologous-WT1 expressing dendritic cells or fibroblasts. These lines were cloned and demonstrated to specifically recognize WT1 transduced autologous fibroblasts but not control transduced fibroblasts by Elispot assays.

To further analyze HLA restriction of these WT1 specific CD8+ T-cell clones, fibroblasts derived from an additional donor (D475), sharing only the HLA-A2 allele with the donor (D349) from which the T-cell clone was established, were transduced with WT1. ELISPOT analysis demonstrated recognition of these D475 target cells by the T-cell clone. To further demonstrate HLA A2 restriction and demonstrate that this epitope is expressed by tumor cells “naturally” overxpressing WT1 (as part of their malignant transformation), the leukemia cell line K562 was tested. K562 was transduced with the HLA A2 molecule, and HLA-A2 negative K562 cells were used as controls for nonspecific IFN-γ release. ELISPOT analysis demonstrated that the T cells recognized the A2 positive K562 cell line, but not the A2 negative K562 cells. Further proof of specificity and HLA-A2 restriction of the recognition was documented by HLA-A2 antibody blocking experiments.

To further define the WT1 epitope, 4 truncated WT1 retroviral constructs were generated. Donor 475 fibroblasts were then transduced with these constructs. ELISPOT assays demonstrated recognition of D475 fibroblasts transduced with the WT1 Tr1 construct (aa2-aa92), thus demonstrating that the WT1 epitope is localized within the first 91 N-terminal amino acids of the WT1 protein. To fine map the epitope, 15mer peptides of the WT1 protein, overlapping by 11 amino acids, were synthesized. The WT1 specific T-cell clone recognized two overlapping 15mer peptides, peptide 9 (QWAPVLDFAPPGASA) (SEQ ID NO: 412) and peptide 10 (VLDFAPPGASAYGSL) (SEQ ID NO: 413). To further characterize the minimal epitope recognized, shared 9mer and 10mer peptides of the 15mers (5 total) were used to analyse the specificity of the clone. The clone specifically recognized the 9mer, VLDFAPPGA (SEQ ID NO:241), and the 10mer, VLDFAPPGAS (SEQ ID NO:411).

### Example 25

### Cloning and Sequencing of TCR Alpha and Beta Chains Derived from a CD8 T Cell Specific for WT1

T cell receptor (TCR) alpha and beta chains from CD8+ T cell clones specific for WT1 are cloned. Sequence analysis is carried to demonstrate the family origin of the the alpha and beta chains of the TCR. Additionally, unique diversity and joining segments (contributing to the specificity of the response) are identified.

Total mRNA from 2×10⁶ cells from a WT1 specific CD8+ T cell clone is isolated using Trizol reagent and cDNA is synthesized using Ready-to-go kits (Pharmacia). To determine Vα and Vβ sequences in a clone, a panel of Vα and Vβ subtype specific primers are synthesized (based on primer sequences generated by Clontech, Palo Alto, Calif.) and used in RT-PCR reactions with cDNA generated from each clone. The RT-PCR reactions demonstrate which Vβ and Vα sequence is expressed by each clone.

To clone the full-length TCR alpha and beta chains from a clone, primers are designed that span the initiator and terminator-coding TCR nucleotides. Standard 35 cycle RT-PCR reactions are established using cDNA synthesized from the CTL clone and the above primers using the proofreading thermostable polymerase PWO (Roche, Basel, Switzerland). The resultant specific bands (˜850 bp for alpha and ˜950 for beta) are ligated into the PCR blunt vector (Invitrogen, Carlsbad, Calif.) and transformed into E. coli. E. coli transformed with plasmids containing full-length alpha and beta chains are identified, and large scale preparations of the corresponding plasmids are generated. Plasmids containing full-length TCR alpha and beta chains are then sequenced using standard methods. The diversity-joining (DJ) region that contributes to the specificity of the TCR is thus determined.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) sequences provided in SEQ ID NOs:327-331, 337-341, and 377-390;
(b) complements of the sequences provided in SEQ ID NOs:327-331, 337-341, and 377-390;
(c) sequences consisting of at least 20 contiguous residues of a sequence provided in SEQ ID NOs:327-331, 337-341, and 377-390;
(d) sequences that hybridize to a sequence provided in SEQ ID NOs:327-331, 337-341, and 377-390, under moderately stringent conditions;
(e) sequences having at least 75% identity to a sequence of SEQ ID NOs:327-331, 337-341, and 377-390;
(f) sequences having at least 90% identity to a sequence of SEQ ID NOs:327-331, 337-341, and 377-390; and
(g) degenerate variants of a sequence provided in SEQ ID NOs:327-331, 337-341, and 377-390.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1; and
(c) sequences having at least 90% identity to a sequence encoded by a polynucleotide of claim 1;
(d) sequences set forth in SEQ ID NOs:241, 332-336, 342-346, 391-395, and 404-413;
(e) sequences having at least 70% identity to a sequence set forth in SEQ ID NOs:241, 332-336, 342-346, 391-395, and 404-413; and
(f) sequences having at least 90% identity to a sequence set forth in SEQ ID NOs:241, 332-336, 342-346, 391-395, and 404-413;

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NOs:327-331, 337-341, and 377-390 under moderately stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polynucleotide according to claim 1, under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells, and thereby inhibiting the development of a cancer in the patient.

18. A composition comprising a WT1 polypeptide resuspended in a buffer comprising at least one sugar selected from the group consisting of trehalose, maltose, sucrose, fructose, and glucose, at a concentration of between about 7 and about 13 %.

19. The composition of claim 18 wherein said concentration is between about 8 and about 12%.

20. The composition of claim 18 wherein said concentration is about 10%.

21. A composition comprising a WT1 polypeptide resuspended in a buffer comprising at least 2 sugars selected from the group consisting of trehalose, maltose, sucrose, fructose, and glucose, at a concentration of between about 7 and about 13 %.

22. The composition of claim 21 wherein said concentration is between about 8 and about 12%.

23. The composition of claim 21 wherein said concentration is about 10%.

24. A composition comprising a WT1 polypeptide resuspended in a buffer comprising at least 3 sugars selected from the group consisting of trehalose, maltose, sucrose, fructose, and glucose, at a concentration of between about 7 and about 13 %.

25. The composition of claim 24 wherein said concentration is between about 8 and about 12%.

26. The composition of claim 24 wherein said concentration is about 10%.

27. A composition comprising a WT1 polypeptide resuspended in a buffer comprising:
(a) at least one sugar selected from the group consisting of trehalose, maltose, sucrose, fructose, and glucose, at a concentration of between about 7 and about 13 %;
(b) ethanolamine;
(c) cysteine; and
(d) Polysorbate-80.

28. The composition of claim 27 wherein said concentration is between about 8 and about 12%.

29. The composition of claim 27 wherein said concentration is about 10%.

30. A composition according to any one of claims 18-29 wherein the WT1 polypeptide comprises an Ra12-WT1 fusion polypeptide.

31. A composition comprising a WT1 polypeptide and MPL-SE.

32. The composition of claim 31 wherein the WT1 polypeptide comprises an Ra12-WT1 fusion polypeptide.

33. A composition comprising a WT1 polypeptide and Enhanzyn.

34. The composition of claim 33 wherein the WT1 polypeptide comprises an Ra12-WT1 fusion polypeptide.


SEQUENCE LISTING (413 sequences)

SEQ ID NO: 1 - protein, 17 aa, Homo sapien
RDLNALLPAVPSLGGGG

SEQ ID NO: 2 - protein, 23 aa, Homo sapien
PSQASSGQARMFPNAPYLPSCLE

SEQ ID NO: 3 - protein, 23 aa, Mus musculus
PSQASSGQARMFPNAPYLPSCLE

SEQ ID NO: 4 - protein, 19 aa, Homo sapien
GATLKGVAAGSSSSVKWTE

SEQ ID NO: 5 - DNA, 22 bp, Homo sapien
gagagtcagacttgaaagcagt

SEQ ID NO: 6 - DNA, 20 bp, Homo sapien
ctgagcctcagcaaatgggc

SEQ ID NO: 7 - DNA, 27 bp, Homo sapien
gagcatgcatgggctccgacgtgcggg

SEQ ID NO: 8 - DNA, 25 bp, Homo sapien
ggggtacccactgaacggtccccga

SEQ ID NO: 9 - DNA, 18 bp, Mus musculus
tccgagccgcacctcatg

SEQ ID NO: 10 - DNA, 18 bp, Mus musculus
gcctgggatgctggactg

SEQ ID NO: 11 - DNA, 27 bp, Mus musculus
gagcatgcgatgggttccgacgtgcgg

SEQ ID NO: 12 - DNA, 29 bp, Mus musculus
ggggtacctcaaagcgccacgtggagttt

SEQ ID NO: 13 - protein, 17 aa, Mus musculus
RDLNALLPAVSSLGGGG

SEQ ID NO: 14 - protein, 19 aa, Mus musculus
GATLKGMAAGSSSSVKWTE

SEQ ID NO: 15 - protein, 15 aa, Homo sapien
RIHTHGVFRGIQDVR

SEQ ID NO: 16 - protein, 15 aa, Mus musculus
RIHTHGVFRGIQDVR

SEQ ID NO: 17 - protein, 14 aa, Mus musculus
VRRVSGVAPTLVRS

SEQ ID NO: 18 - protein, 14 aa, Homo sapien
VRRVPGVAPTLVRS

SEQ ID NO: 19 - protein, 15 aa, Homo sapien
CQKKFARSDELVRHH

SEQ ID NO: 20 - protein, 15 aa, Mus musculus
CQKKFARSDELVRHH

SEQ ID NO: 21 - DNA, 21 bp, Mus musculus
cccaggctgcaataagagata

SEQ ID NO: 22 - DNA, 21 bp, Mus musculus
atgttgtgatggcggaccaat

SEQ ID NO: 23 - DNA, 20 bp, Homo sapien
gtggggcgccccaggcacca

SEQ ID NO: 24 - DNA, 24 bp, Homo sapien
gtccttaatgctacgcacgatttc

SEQ ID NO: 25 - DNA, 21 bp, Homo sapien
ggcatctgagaccagtgagaa

SEQ ID NO: 26 - DNA, 21 bp, Homo sapien
gctgtcccacttacagatgca

SEQ ID NO: 27 - DNA, 21 bp, Homo sapien
tcaaagcgccagctggagttt

SEQ ID NO: 28 - protein, 9 aa, Homo sapien
AAGSSSSVK

SEQ ID NO: 29 - protein, 9 aa, Homo sapien
AAQFPNHSF

SEQ ID NO: 30 - protein, 9 aa, Homo sapien
AEPHEEQCL

SEQ ID NO: 31 - protein, 9 aa, Homo sapien
AGACRYGPF

SEQ ID NO: 32 - protein, 9 aa, Homo sapien
AGSSSSVKW

SEQ ID NO: 33 - protein, 9 aa, Homo sapien
AIRNQGYST

SEQ ID NO: 34 - protein, 9 aa, Homo sapien
ALLPAVPSL

SEQ ID NO: 35 - protein, 9 aa, Homo sapien
ALLPAVSSL

SEQ ID NO: 36 - protein, 9 aa, Homo sapien
AQFPNHSFK

SEQ ID NO: 37 - protein, 9 aa, Homo sapien
AQWAPVLDF

SEQ ID NO: 38 - protein, 9 aa, Homo sapien
ARMFPNAPY

SEQ ID NO: 39 - protein, 9 aa, Homo sapien
ARSDELVRH

SEQ ID NO: 40 - protein, 9 aa, Homo sapien
ASSGQARMF

SEQ ID NO: 41 - protein, 9 aa, Homo sapien
AYGSLGGPA

SEQ ID NO: 42 - protein, 9 aa, Homo sapien
AYPGCNKRY

SEQ ID NO: 43 - protein, 9 aa, Homo sapien
CALPVSGAA

SEQ ID NO: 44 - protein, 9 aa, Homo sapien
CAYPGCNKR

SEQ ID NO: 45 - protein, 9 aa, Homo sapien
CHTPTDSCT

SEQ ID NO: 46 - protein, 9 aa, Homo sapien
CKTCQRKFS

SEQ ID NO: 47 - protein, 9 aa, Homo sapien
CLESQPAIR

SEQ ID NO: 48 - protein, 9 aa, Homo sapien
CLSAFTVHF

SEQ ID NO: 49 - protein, 9 aa, Homo sapien
CMTWNQMNL

SEQ ID NO: 50 - protein, 9 aa, Homo sapien
CRWPSCQKK

SEQ ID NO: 51 - protein, 9 aa, Homo sapien
CRYGPFGPP

SEQ ID NO: 52 - protein, 9 aa, Homo sapien
CTGSQALLL

SEQ ID NO: 53 - protein, 9 aa, Homo sapien
DELVRHHNM

SEQ ID NO: 54 - protein, 9 aa, Homo sapien
DFAPPGASA

SEQ ID NO: 55 - protein, 9 aa, Homo sapien
DFKDCERRF

SEQ ID NO: 56 - protein, 9 aa, Homo sapien
DGTPSYGHT

SEQ ID NO: 57 - protein, 9 aa, Homo sapien
DHLKTHTRT

SEQ ID NO: 58 - protein, 9 aa, Homo sapien
DLNALLPAV

SEQ ID NO: 59 - protein, 9 aa, Homo sapien
DPMGQQGSL

SEQ ID NO: 60 - protein, 9 aa, Homo sapien
DQLKRHQRR

SEQ ID NO: 61 - protein, 9 aa, Homo sapien
DSCTGSQAL

SEQ ID NO: 62 - protein, 9 aa, Homo sapien
DVRDLNALL

SEQ ID NO: 63 - protein, 9 aa, Homo sapien
DVRRVPGVA

SEQ ID NO: 64 - protein, 9 aa, Homo sapien
EDPMGQQGS

SEQ ID NO: 65 - protein, 9 aa, Homo sapien
EEQCLSAFT

SEQ ID NO: 66 - protein, 9 aa, Homo sapien
EKPYQCDFK

SEQ ID NO: 67 - protein, 9 aa, Homo sapien
EKRPFMCAY

SEQ ID NO: 68 - protein, 9 aa, Homo sapien
EPHEEQCLS

SEQ ID NO: 69 - protein, 9 aa, Homo sapien
EQCLSAFTV

SEQ ID NO: 70 - protein, 9 aa, Homo sapien
ESDNHTAPI

SEQ ID NO: 71 - protein, 9 aa, Homo sapien
ESDNHTTPI

SEQ ID NO: 72 - protein, 9 aa, Homo sapien
ESQPAIRNQ

SEQ ID NO: 73 - protein, 9 aa, Homo sapien
ETSEKRPFM

SEQ ID NO: 74 - protein, 9 aa, Homo sapien
FAPPGASAY

SEQ ID NO: 75 - protein, 9 aa, Homo sapien
FARSDELVR

SEQ ID NO: 76 - protein, 9 aa, Homo sapien
FGPPPPSQA

SEQ ID NO: 77 - protein, 9 aa, Homo sapien
FKDCERRFS

SEQ ID NO: 78 - protein, 9 aa, Homo sapien
FKLSHLQMH

SEQ ID NO: 79 - protein, 9 aa, Homo sapien
FPNAPYLPS

SEQ ID NO: 80 - protein, 9 aa, Homo sapien
FQCKTCQRK

SEQ ID NO: 81 - protein, 9 aa, Homo sapien
FRGIQDVRR

SEQ ID NO: 82 - protein, 9 aa, Homo sapien
FSGQFTGTA

SEQ ID NO: 83 - protein, 9 aa, Homo sapien
FSRSDQLKR

SEQ ID NO: 84 - protein, 9 aa, Homo sapien
FTGTAGACR

SEQ ID NO: 85 - protein, 9 aa, Homo sapien
FTVHFSGQF

SEQ ID NO: 86 - protein, 9 aa, Homo sapien
GAAQWAPVL

SEQ ID NO: 87 - protein, 9 aa, Homo sapien
GAEPHEEQC

SEQ ID NO: 88 - protein, 9 aa, Homo sapien
GATLKGVAA

SEQ ID NO: 89 - protein, 9 aa, Homo sapien
GCALPVSGA

SEQ ID NO: 90 - protein, 9 aa, Homo sapien
GCNKRYFKL

SEQ ID NO: 91 - protein, 9 aa, Homo sapien
GEKPYQCDF

SEQ ID NO: 92 - protein, 9 aa, Homo sapien
GGGGCALPV

SEQ ID NO: 93 - protein, 9 aa, Homo sapien
GGPAPPPAP

SEQ ID NO: 94 - protein, 9 aa, Homo sapien
GHTPSHHAA

SEQ ID NO: 95 - protein, 9 aa, Homo sapien
GKTSEKPFS

SEQ ID NO: 96 - protein, 9 aa, Homo sapien
GPFGPPPPS

SEQ ID NO: 97 - protein, 9 aa, Homo sapien
GPPPPSQAS

SEQ ID NO: 98 - protein, 9 aa, Homo sapien
GQARMFPNA

SEQ ID NO: 99 - protein, 9 aa, Homo sapien
GQFTGTAGA

SEQ ID NO: 100 - protein, 9 aa, Homo sapien
GQSNHSTGY

SEQ ID NO: 101 - protein, 9 aa, Homo sapien
GSDVRDLNA

SEQ ID NO: 102 - protein, 9 aa, Homo sapien
GSQALLLRT

SEQ ID NO: 103 - protein, 9 aa, Homo sapien
GVFRGIQDV

SEQ ID NO: 104 - protein, 9 aa, Homo sapien
GVKPFQCKT

SEQ ID NO: 105 - protein, 9 aa, Homo sapien
GYESDNHTA

SEQ ID NO: 106 - protein, 9 aa, Homo sapien
GYESDNHTT

SEQ ID NO: 107 - protein, 9 aa, Homo sapien
HEEQCLSAF

SEQ ID NO: 108 - protein, 9 aa, Homo sapien
HHNMHQRNM

SEQ ID NO: 109 - protein, 9 aa, Homo sapien
HQRRHTGVK

SEQ ID NO: 110 - protein, 9 aa, Homo sapien
HSFKHEDPM

SEQ ID NO: 111 - protein, 9 aa, Homo sapien
HSRKHTGEK

SEQ ID NO: 112 - protein, 9 aa, Homo sapien
HTGEKPYQC

SEQ ID NO: 113 - protein, 9 aa, Homo sapien
HTHGVFRGI

SEQ ID NO: 114 - protein, 9 aa, Homo sapien
HTRTHTGKT

SEQ ID NO: 115 - protein, 9 aa, Homo sapien
HTTPILCGA

SEQ ID NO: 116 - protein, 9 aa, Homo sapien
ILCGAQYRI

SEQ ID NO: 117 - protein, 9 aa, Homo sapien
IRNQGYSTV

SEQ ID NO: 118 - protein, 9 aa, Homo sapien
KDCERRFSR

SEQ ID NO: 119 - protein, 9 aa, Homo sapien
KFARSDELV

SEQ ID NO: 120 - protein, 9 aa, Homo sapien
KFSRSDHLK

SEQ ID NO: 121 - protein, 9 aa, Homo sapien
KHEDPMGQQ

SEQ ID NO: 122 - protein, 9 aa, Homo sapien
KKFARSDEL

SEQ ID NO: 123 - protein, 9 aa, Homo sapien
KPFSCRWPS

SEQ ID NO: 124 - protein, 9 aa, Homo sapien
KPYQCDFKD

SEQ ID NO: 125 - protein, 9 aa, Homo sapien
KQEPSWGGA

SEQ ID NO: 126 - protein, 9 aa, Homo sapien
KRHQRRHTG

SEQ ID NO: 127 - protein, 9 aa, Homo sapien
KRYFKLSHL

SEQ ID NO: 128 - protein, 9 aa, Homo sapien
KTCQRKFSR

SEQ ID NO: 129 - protein, 9 aa, Homo sapien
KTSEKPFSC

SEQ ID NO: 130 - protein, 9 aa, Homo sapien
LDFAPPGAS

SEQ ID NO: 131 - protein, 9 aa, Homo sapien
LECMTWNQM

SEQ ID NO: 132 - protein, 9 aa, Homo sapien
LESQPAIRN

SEQ ID NO: 133 - protein, 9 aa, Homo sapien
LGATLKGVA

SEQ ID NO: 134 - protein, 9 aa, Homo sapien
LGGGGGCAL

SEQ ID NO: 135 - protein, 9 aa, Homo sapien
LKGVAAGSS

SEQ ID NO: 136 - protein, 9 aa, Homo sapien
LKRHQRRHT

SEQ ID NO: 137 - protein, 9 aa, Homo sapien
LKTHTRTHT

SEQ ID NO: 138 - protein, 9 aa, Homo sapien
LPVSGAAQW

SEQ ID NO: 139 - protein, 9 aa, Homo sapien
LQMHSRKHT

SEQ ID NO: 140 - protein, 9 aa, Homo sapien
LRTPYSSDN

SEQ ID NO: 141 - protein, 9 aa, Homo sapien
LSHLQMHSR

SEQ ID NO: 142 - protein, 9 aa, Homo sapien
MCAYPGCNK

SEQ ID NO: 143 - protein, 9 aa, Homo sapien
MHQRNMTKL

SEQ ID NO: 144 - protein, 9 aa, Homo sapien
NAPYLPSCL

SEQ ID NO: 145 - protein, 9 aa, Homo sapien
NKRYFKLSH

SEQ ID NO: 146 - protein, 9 aa, Homo sapien
NLGATLKGV

SEQ ID NO: 147 - protein, 9 aa, Homo sapien
NLYQMTSQL

SEQ ID NO: 148 - protein, 9 aa, Homo sapien
NMHQRNMTK

SEQ ID NO: 149 - protein, 9 aa, Homo sapien
NMTKLQLAL

SEQ ID NO: 150 - protein, 9 aa, Homo sapien
NQGYSTVTF

SEQ ID NO: 151 - protein, 9 aa, Homo sapien
NQMNLGATL

SEQ ID NO: 152 - protein, 9 aa, Homo sapien
PAIRNQGYS

SEQ ID NO: 153 - protein, 9 aa, Homo sapien
PGASAYGSL

SEQ ID NO: 154 - protein, 9 aa, Homo sapien
PHEEQCLSA

SEQ ID NO: 155 - protein, 9 aa, Homo sapien
PILCGAQYR

SEQ ID NO: 156 - protein, 9 aa, Homo sapien
PPPPHSFIK

SEQ ID NO: 157 - protein, 9 aa, Homo sapien
PPPPPHSFI

SEQ ID NO: 158 - protein, 9 aa, Homo sapien
PPPPPPHSF

SEQ ID NO: 159 - protein, 9 aa, Homo sapien
PSCQKKFAR

SEQ ID NO: 160 - protein, 9 aa, Homo sapien
QALLLRTPY

SEQ ID NO: 161 - protein, 9 aa, Homo sapien
QASSGQARM

SEQ ID NO: 162 - protein, 9 aa, Homo sapien
QCDFKDCER

SEQ ID NO: 163 - protein, 9 aa, Homo sapien
QCKTCQRKF

SEQ ID NO: 164 - protein, 9 aa, Homo sapien
QDVRRVPGV

SEQ ID NO: 165 - protein, 9 aa, Homo sapien
QFTGTAGAC

SEQ ID NO: 166 - protein, 9 aa, Homo sapien
QGSLGEQQY

SEQ ID NO: 167 - protein, 9 aa, Homo sapien
QLECMTWNQ

SEQ ID NO: 168 - protein, 9 aa, Homo sapien
QMNLGATLK

SEQ ID NO: 169 - protein, 9 aa, Homo sapien
QMTSQLECM

SEQ ID NO: 170 - protein, 9 aa, Homo sapien
QPAIRNQGY

SEQ ID NO: 171 - protein, 9 aa, Homo sapien
QQYSVPPPV

SEQ ID NO: 172 - protein, 9 aa, Homo sapien
QRKFSRSDH

SEQ ID NO: 173 - protein, 9 aa, Homo sapien
QRNMTKLQL

SEQ ID NO: 174 - protein, 9 aa, Homo sapien
QWAPVLDFA

SEQ ID NO: 175 - protein, 9 aa, Homo sapien
QYRIHTHGV

SEQ ID NO: 176 - protein, 9 aa, Homo sapien
QYSVPPPVY

SEQ ID NO: 177 - protein, 9 aa, Homo sapien
RDLNALLPA

SEQ ID NO: 178 - protein, 9 aa, Homo sapien
RFSRSDQLK

SEQ ID NO: 179 - protein, 9 aa, Homo sapien
RGIQDVRRV

SEQ ID NO: 180 - protein, 9 aa, Homo sapien
RHHNMHQRN

SEQ ID NO: 181 - protein, 9 aa, Homo sapien
RHQRRHTGV

SEQ ID NO: 182 - protein, 9 aa, Homo sapien
RIHTHGVFR

SEQ ID NO: 183 - protein, 9 aa, Homo sapien
RKFSRSDHL

SEQ ID NO: 184 - protein, 9 aa, Homo sapien
RKHTGEKPY

SEQ ID NO: 185 - protein, 9 aa, Homo sapien
RMFPNAPYL

SEQ ID NO: 186 - protein, 9 aa, Homo sapien
RNMTKLQLA

SEQ ID NO: 187 - protein, 9 aa, Homo sapien
RRFSRSDQL

SEQ ID NO: 188 - protein, 9 aa, Homo sapien
RRHTGVKPF

SEQ ID NO: 189 - protein, 9 aa, Homo sapien
RRVPGVAPT

SEQ ID NO: 190 - protein, 9 aa, Homo sapien
RSASETSEK

SEQ ID NO: 191 - protein, 9 aa, Homo sapien
RSDELVRHH

SEQ ID NO: 192 - protein, 9 aa, Homo sapien
RSDHLKTHT

SEQ ID NO: 193 - protein, 9 aa, Homo sapien
RSDQLKRHQ

SEQ ID NO: 194 - protein, 9 aa, Homo sapien
RTPYSSDNL

SEQ ID NO: 195 - protein, 9 aa, Homo sapien
RVPGVAPTL

SEQ ID NO: 196 - protein, 9 aa, Homo sapien
RWPSCQKKF

SEQ ID NO: 197 - protein, 9 aa, Homo sapien
SASETSEKR

SEQ ID NO: 198 - protein, 9 aa, Homo sapien
SCLESQPAI

SEQ ID NO: 199 - protein, 9 aa, Homo sapien
SCLESQPTI

SEQ ID NO: 200 - protein, 9 aa, Homo sapien
SCQKKFARS

SEQ ID NO: 201 - protein, 9 aa, Homo sapien
SCRWPSCQK

SEQ ID NO: 202 - protein, 9 aa, Homo sapien
SCTGSQALL

SEQ ID NO: 203 - protein, 9 aa, Homo sapien
SDELVRHHN

SEQ ID NO: 204 - protein, 9 aa, Homo sapien
SDNHTTPIL

SEQ ID NO: 205 - protein, 9 aa, Homo sapien
SDNLYQMTS

SEQ ID NO: 206 - protein, 9 aa, Homo sapien
SDVRDLNAL

SEQ ID NO: 207 - protein, 9 aa, Homo sapien
SEKPFSCRW

SEQ ID NO: 208 - protein, 9 aa, Homo sapien
SEKRPFMCA

SEQ ID NO: 209 - protein, 9 aa, Homo sapien
SETSEKRPF

SEQ ID NO: 210 - protein, 9 aa, Homo sapien
SFIKQEPSW

SEQ ID NO: 211 - protein, 9 aa, Homo sapien
SGAAQWAPV

SEQ ID NO: 212 - protein, 9 aa, Homo sapien
SGQARMFPN

SEQ ID NO: 213 - protein, 9 aa, Homo sapien
SHHAAQFPN

SEQ ID NO: 214 - protein, 9 aa, Homo sapien
SLGEQQYSV

SEQ ID NO: 215 - protein, 9 aa, Homo sapien
SLGGGGGCA

SEQ ID NO: 216 - protein, 9 aa, Homo sapien
SQASSGQAR

SEQ ID NO: 217 - protein, 9 aa, Homo sapien
SSDNLYQMT

SEQ ID NO: 218 - protein, 9 aa, Homo sapien
SVPPPVYGC

SEQ ID NO: 219 - protein, 9 aa, Homo sapien
TCQRKFSRS

SEQ ID NO: 220 - protein, 9 aa, Homo sapien
TDSCTGSQA

SEQ ID NO: 221 - protein, 9 aa, Homo sapien
TEGQSNHST

SEQ ID NO: 222 - protein, 9 aa, Homo sapien
TGKTSEKPF

SEQ ID NO: 223 - protein, 9 aa, Homo sapien
TGSQALLLR

SEQ ID NO: 224 - protein, 9 aa, Homo sapien
TGTAGACRY

SEQ ID NO: 225 - protein, 9 aa, Homo sapien
TGYESDNHT

SEQ ID NO: 226 - protein, 9 aa, Homo sapien
TLVRSASET

SEQ ID NO: 227 - protein, 9 aa, Homo sapien
TPILCGAQY

SEQ ID NO: 228 - protein, 9 aa, Homo sapien
TPSHHAAQF

SEQ ID NO: 229 - protein, 9 aa, Homo sapien
TPSYGHTPS

SEQ ID NO: 230 - protein, 9 aa, Homo sapien
TPTDSCTGS

SEQ ID NO: 231 - protein, 9 aa, Homo sapien
TPYSSDNLY

SEQ ID NO: 232 - protein, 9 aa, Homo sapien
TSEKPFSCR

SEQ ID NO: 233 - protein, 9 aa, Homo sapien
TSEKRPFMC

SEQ ID NO: 234 - protein, 9 aa, Homo sapien
TSQLECMTW

SEQ ID NO: 235 - protein, 9 aa, Homo sapien
TVHFSGQFT

SEQ ID NO: 236 - protein, 9 aa, Homo sapien
VAAGSSSSV

SEQ ID NO: 237 - protein, 9 aa, Homo sapien
VAPTLVRSA

SEQ ID NO: 238 - protein, 9 aa, Homo sapien
VFRGIQDVR

SEQ ID NO: 239 - protein, 9 aa, Homo sapien
VKPFQCKTC

SEQ ID NO: 240 - protein, 9 aa, Homo sapien
VKWTEGQSN

SEQ ID NO: 241 - protein, 9 aa, Homo sapien
VLDFAPPGA

SEQ ID NO: 242 - protein, 9 aa, Homo sapien
VPGVAPTLV

SEQ ID NO: 243 - protein, 9 aa, Homo sapien
VRHHNMHQR

SEQ ID NO: 244 - protein, 9 aa, Homo sapien
VTFDGTPSY

SEQ ID NO: 245 - protein, 9 aa, Homo sapien
WNQMNLGAT

SEQ ID NO: 246 - protein, 9 aa, Homo sapien
WPSCQKKFA

SEQ ID NO: 247 - protein, 9 aa, Homo sapien
WTEGQSNHS

SEQ ID NO: 248 - protein, 9 aa, Homo sapien
YFKLSHLQM

SEQ ID NO: 249 - protein, 9 aa, Homo sapien
YGHTPSHHA

SEQ ID NO: 250 - protein, 9 aa, Homo sapien
YPGCNKRYF

SEQ ID NO: 251 - protein, 9 aa, Homo sapien
YQMTSQLEC

SEQ ID NO: 252 - protein, 9 aa, Homo sapien
YRIHTHGVF

SEQ ID NO: 253 - protein, 9 aa, Homo sapien
YSSDNLYQM

SEQ ID NO: 254 - protein, 9 aa, Mus musculus
AEPHEEQCL

SEQ ID NO: 255 - protein, 9 aa, Mus musculus
ALLPAVSSL

SEQ ID NO: 256 - protein, 9 aa, Mus musculus
AYGSLGGPA

SEQ ID NO: 257 - protein, 9 aa, Mus musculus
AYPGCNKRY

SEQ ID NO: 258 - protein, 9 aa, Mus musculus
CMTWNQMNL

SEQ ID NO: 259 - protein, 9 aa, Mus musculus
CTGSQALLL

SEQ ID NO: 260 - protein, 9 aa, Mus musculus
DGAPSYGHT

SEQ ID NO: 261 - protein, 9 aa, Mus musculus
DLNALLPAV

SEQ ID NO: 262 - protein, 9 aa, Mus musculus
DPMGQQGSL

SEQ ID NO: 263 - protein, 9 aa, Mus musculus
DSCTGSQAL

SEQ ID NO: 264 - protein, 9 aa, Mus musculus
DVRDLNALL

SEQ ID NO: 265 - protein, 9 aa, Mus musculus
EQCLSAFTL

SEQ ID NO: 266 - protein, 9 aa, Mus musculus
ESDNHTAPI

SEQ ID NO: 267 - protein, 9 aa, Mus musculus
FPNAPYLPS

SEQ ID NO: 268 - protein, 9 aa, Mus musculus
GCNKRYFKL

SEQ ID NO: 269 - protein, 9 aa, Mus musculus
GQARMFPNA

SEQ ID NO: 270 - protein, 9 aa, Mus musculus
GVFRGIQDV

SEQ ID NO: 271 - protein, 9 aa, Mus musculus
GYESDNHTA

SEQ ID NO: 272 - protein, 9 aa, Mus musculus
HSFKHEDPM

SEQ ID NO: 273 - protein, 9 aa, Mus musculus
HTHGVFRGI

SEQ ID NO: 274 - protein, 9 aa, Mus musculus
ILCGAQYRI

SEQ ID NO: 275 - protein, 9 aa, Mus musculus
KFARSDELV

SEQ ID NO: 276 - protein, 9 aa, Mus musculus
KRYFKLSHL

SEQ ID NO: 277 - protein, 9 aa, Mus musculus
KTSEKPFSC

SEQ ID NO: 278 - protein, 9 aa, Mus musculus
LECMTWNQM

SEQ ID NO: 279 - protein, 9 aa, Mus musculus
LGGGGGCGL

SEQ ID NO: 280 - protein, 9 aa, Mus musculus
LQMHSRKHT

SEQ ID NO: 281 - protein, 9 aa, Mus musculus
MHQRNMTKL

SEQ ID NO: 282 - protein, 9 aa, Mus musculus
NAPYLPSCL

SEQ ID NO: 283 - protein, 9 aa, Mus musculus
NLGATLKGM

SEQ ID NO: 284 - protein, 9 aa, Mus musculus
NLYQMTSQL

SEQ ID NO: 285 - protein, 9 aa, Mus musculus
NMTKLHVAL

SEQ ID NO: 286 - protein, 9 aa, Mus musculus
NQMNLGATL

SEQ ID NO: 287 - protein, 9 aa, Mus musculus
PGASAYGSL

SEQ ID NO: 288 - protein, 9 aa, Mus musculus
QASSGQARM

SEQ ID NO: 289 - protein, 9 aa, Mus musculus
QMTSQLECM

SEQ ID NO: 290 - protein, 9 aa, Mus musculus
QQYSVPPPV

SEQ ID NO: 291 - protein, 9 aa, Mus musculus
QYRIHTHGV

SEQ ID NO: 292 - protein, 9 aa, Mus musculus
QYSVPPPVY

SEQ ID NO: 293 - protein, 9 aa, Mus musculus
RMFPNAPYL

SEQ ID NO: 294 - protein, 9 aa, Mus musculus
RTPYSSDNL

SEQ ID NO: 295 - protein, 9 aa, Mus musculus
RVSGVAPTL

SEQ ID NO: 296 - protein, 9 aa, Mus musculus
SCLESQPTI

SEQ ID NO: 297 - protein, 9 aa, Mus musculus
SCQKKFARS

SEQ ID NO: 298 - protein, 9 aa, Mus musculus
SDVRDLNAL

SEQ ID NO: 299 - protein, 9 aa, Mus musculus
SLGEQQYSV

SEQ ID NO: 300 - protein, 9 aa, Mus musculus
TCQRKFSRS

SEQ ID NO: 301 - protein, 9 aa, Mus musculus
TEGQSNHGI

SEQ ID NO: 302 - protein, 9 aa, Mus musculus
TLHFSGQFT

SEQ ID NO: 303 - protein, 9 aa, Mus musculus
TLVRSASET

SEQ ID NO: 304 - protein, 9 aa, Mus musculus
VLDFAPPGA

SEQ ID NO: 305 - protein, 9 aa, Mus musculus
WNQMNLGAT

SEQ ID NO: 306 - protein, 9 aa, Mus musculus
YFKLSHLQM

SEQ ID NO: 307 - protein, 9 aa, Mus musculus
YQMTSQLEC

SEQ ID NO: 308 - protein, 9 aa, Mus musculus
YSSDNLYQM

SEQ ID NO: 309 - protein, 6 aa, Homo sapien
GAAQWA

SEQ ID NO: 310 - protein, 12 aa, Homo sapien
ASAYGSLGGPAP

SEQ ID NO: 311 - protein, 15 aa, Homo sapien
AFTVHFSGQFTGTAG

SEQ ID NO: 312 - protein, 5 aa, Homo sapien
HAAQF

SEQ ID NO: 313 - protein, 32 aa, Homo sapien
CHTPTDSCTGSQALLLRTPYSSDNLYQMTSQL

SEQ ID NO: 314 - protein, 32 aa, Homo sapien
RIHTHGVFRGIQDVRRVPGVAPTLVRSASETS

SEQ ID NO: 315 - protein, 4 aa, Homo sapien
RYFK

SEQ ID NO: 316 - protein, 14 aa, Homo sapien
ERRFSRSDQLKRHQ

SEQ ID NO: 317 - protein, 22 aa, Homo sapien
QRKFSRSDHLKTHTRTHTGKTS

SEQ ID NO: 318 - protein, 21 aa, Homo sapien
CQKKFARSDELVRHHNMHQRN

SEQ ID NO: 319 - protein, 449 aa, Homo sapien
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 320 - protein, 449 aa, Mus musculus
MGSDVRDLNALLPAVSSLGGGGGCGLPVSGAAQWAPVLDFAPPGASAYGSLGGPAPPPAPPPPPPPPPHSFIKQEPSWGGAEPHEEQCLSAFTLHFSGQFTGTAGACRYGPFGPPPPSQASSGQARMFPNAPYLPSCLESQPTIRNQGYSTVTFDGAPSYGHTPSHHAAQFPNHSFKHEDPMGQQGSLGEQQYSVPPPVYGCHTPTDSCTGSQALLLRTPYSSDNLYQMTSQLECMTWNQMNLGATLKGMAAGSSSSVKWTEGQSNHGIGYESDNHTAPILCGAQYRIHTHGVFRGIQDVRRVSGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWHSCQKKFARSDELVRHHNMHQRNMTKLHVAL

SEQ ID NO: 321 - protein, 9 aa, Homo sapien and Mus musculus
PSQASSGQA

SEQ ID NO: 322 - protein, 9 aa, Homo sapien and Mus musculus
SSGQARMFP

SEQ ID NO: 323 - protein, 9 aa, Homo sapien and Mus musculus
QARMFPNAP

SEQ ID NO: 324 - protein, 9 aa, Homo sapien and Mus musculus
MFPNAPYLP

SEQ ID NO: 325 - protein, 9 aa, Homo sapien and Mus musculus
PNAPYLPSC

SEQ ID NO: 326 - protein, 9 aa, Homo sapien and Mus musculus
APYLPSCLE

SEQ ID NO: 327 - DNA, 1029 bp, Homo sapiens
atgcagcatcaccaccatcaccacatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcagcatcaccaccatcaccacgtgtctatcgaaggtcgtgctagctctggtggcagcggtctggttccgcgtggtagctctggttcgggggacgacgacgacaaatctagtaggcacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 328 - DNA, 1233 bp, Homo sapiens
atgcagcatcaccaccatcaccacatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcagcatcaccaccatcaccacgtgtctatcgaaggtcgtgctagctctggtggcagcggtctggttccgcgtggtagctctggttcgggggacgacgacgacaaatctagtaggggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggctga

SEQ ID NO: 329 - DNA, 1776 bp, Homo sapiens
atgcagcatcaccaccatcaccacatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcagcatcaccaccatcaccacgtgtctatcgaaggtcgtgctagctctggtggcagcggtctggttccgcgtggtagctctggttcgggggacgacgacgacaaatctagtaggatgggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 330 - DNA, 771 bp, Homo sapiens
atgcagcatcaccaccatcaccacggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggctga

SEQ ID NO: 331 - DNA, 567 bp, Homo sapiens
atgcagcatcaccaccatcaccaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 332 - protein, 342 aa, Homo sapiens
MQHHHHHHMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMQHHHHHHVSIEGRASSGGSGLVPRGSSGSGDDDDKSSRHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 333 - protein, 410 aa, Homo sapiens
MQHHHHHHMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMQHHHHHHVSIEGRASSGGSGLVPRGSSGSGDDDDKSSRGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKG

SEQ ID NO: 334 - protein, 591 aa, Homo sapiens
MQHHHHHHMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMQHHHHHHVSIEGRASSGGSGLVPRGSSGSGDDDDKSSRMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 335 - protein, 256 aa, Homo sapiens
MQHHHHHHGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKG

SEQ ID NO: 336 - protein, 188 aa, Homo sapiens
MQHHHHHHHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 337 - DNA, 324 bp, Homo sapiens
atgcagcatcaccaccatcaccacggttccgacgtgcgggacctgaacgcactgctgccggcagttccatccctgggtggcggtggaggctgcgcactgccggttagcggtgcagcacagtgggctccagttctggacttcgcaccgcctggtgcatccgcatacggttccctgggtggtccagcacctccgcccgcaacgcccccaccgcctccaccgcccccgcactccttcatcaaacaggaacctagctggggtggtgcagaaccgcacgaagaacagtgcctgagcgcattctgagaattctgcagatatccatcacac

SEQ ID NO: 338 - DNA, 462 bp, Homo sapiens
atgcagcatcaccaccatcaccaccacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttatactgatga

SEQ ID NO: 339 - DNA, 405 bp, Homo sapiens
atgcagcatcaccaccatcaccaccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgatga

SEQ ID NO: 340 - DNA, 339 bp, Homo sapiens
atgcagcatcaccaccatcaccaccacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 341 - DNA, 1110 bp, Homo sapiens
atgcagcatcaccaccatcaccaccactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 342 - protein, 99 aa, Homo sapiens
MQHHHHHHGSDVRDLNALLPAVPSLGGGGGCALPVSGAAQWAPVLDFAPPGASAYGSLGGPAPPPAPPPPPPPPPHSFIKQEPSWGGAEPHEEQCLSAF

SEQ ID NO: 343 - protein, 152 aa, Homo sapiens
MQHHHHHHHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLY

SEQ ID NO: 344 - protein, 133 aa, Homo sapiens
MQHHHHHHQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQ

SEQ ID NO: 345 - protein, 112 aa, Homo sapiens
MQHHHHHHHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 346 - protein, 369 aa, Homo sapiens
MQHHHHHHHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 347 - DNA, 21 bp, Artificial Sequence
  Primer
ggctccgacgtgcgggacctg

SEQ ID NO: 348 - DNA, 30 bp, Artificial Sequence
  Primer
gaattctcaaagcgccagctggagtttggt

SEQ ID NO: 349 - DNA, 21 bp, Artificial Sequence
  Primer
ggctccgacgtgcgggacctg

SEQ ID NO: 350 - DNA, 30 bp, Artificial Sequence; identical to SEQ ID NO: 348
  Primer

SEQ ID NO: 351 - DNA, 21 bp, Artificial Sequence
  Primer
cacagcacagggtacgagagc

SEQ ID NO: 352 - DNA, 30 bp, Artificial Sequence; identical to SEQ ID NO: 348
  Primer

SEQ ID NO: 353 - DNA, 29 bp, Artificial Sequence
  Primer
cacgaagaacagtgcctgagcgcattcac

SEQ ID NO: 354 - DNA, 32 bp, Artificial Sequence
  Primer
ccggcgaattcatcagtataaattgtcactgc

SEQ ID NO: 355 - DNA, 24 bp, Artificial Sequence
  Primer
caggctttgctgctgaggacgccc

SEQ ID NO: 356 - DNA, 34 bp, Artificial Sequence
  Primer
cacggagaattcatcactggtatggtttctcacc

SEQ ID NO: 357 - DNA, 28 bp, Artificial Sequence
  Primer
cacagcaggaagcacactggtgagaaac

SEQ ID NO: 358 - DNA, 30 bp, Artificial Sequence
  Primer
ggatatctgcagaattctcaaagcgccagc

SEQ ID NO: 359 - DNA, 22 bp, Artificial Sequence
  Primer
cactccttcatcaaacaggaac

SEQ ID NO: 360 - DNA, 30 bp, Artificial Sequence; identical to SEQ ID NO: 358
  Primer

SEQ ID NO: 361 - DNA, 33 bp, Artificial Sequence
  Primer
ggttccgacgtgcgggacctgaacgcactgctg

SEQ ID NO: 362 - DNA, 40 bp, Artificial Sequence
  Primer
ctgccggcagcagtgcgttcaggtcccgcacgtcggaacc

SEQ ID NO: 363 - DNA, 35 bp, Artificial Sequence
  Primer
ccggcagttccatccctgggtggcggtggaggctg

SEQ ID NO: 364 - DNA, 38 bp, Artificial Sequence
  Primer
cggcagtgcgcagcctccaccgccacccagggatggaa

SEQ ID NO: 365 - DNA, 35 bp, Artificial Sequence
  Primer
cgcactgccggttagcggtgcagcacagtgggctc

SEQ ID NO: 366 - DNA, 33 bp, Artificial Sequence
  Primer
cagaactggagcccactgtgctgcaccgctaac

SEQ ID NO: 367 - DNA, 38 bp, Artificial Sequence
  Primer
cagttctggacttcgcaccgcctggtgcatccgcatac

SEQ ID NO: 368 - DNA, 39 bp, Artificial Sequence
  Primer
cagggaaccgtatgcggatgcaccaggcggtgcgaagtc

SEQ ID NO: 369 - DNA, 38 bp, Artificial Sequence
  Primer
ggttccctgggtggtccagcacctccgcccgcaacgcc

SEQ ID NO: 370 - DNA, 38 bp, Artificial Sequence
  Primer
ggcggtgggggcgttgcgggcggaggtgctggaccacc

SEQ ID NO: 371 - DNA, 40 bp, Artificial Sequence
  Primer
cccaccgcctccaccgcccccgcactccttcatcaaacag

SEQ ID NO: 372 - DNA, 39 bp, Artificial Sequence
  Primer
ctaggttcctgtttgatgaaggagtgcgggggcggtgga

SEQ ID NO: 373 - DNA, 38 bp, Artificial Sequence
  Primer
gaacctagctggggtggtgcagaaccgcacgaagaaca

SEQ ID NO: 374 - DNA, 39 bp, Artificial Sequence
  Primer
ctcaggcactgttcttcgtgcggttctgcaccaccccag

SEQ ID NO: 375 - DNA, 32 bp, Artificial Sequence
  Primer
gtgcctgagcgcattctgagaattctgcagat

SEQ ID NO: 376 - DNA, 34 bp, Artificial Sequence
  Primer
gtgtgatggatatctgcagaattctcagaatgcg

SEQ ID NO: 377 - DNA, 1292 bp, Homo sapiens
  misc_feature 253,256,517,518,520,521,522,743,753,754,758: n = A,T,C or G
atgggctccgacgttcgtgacctgagcgcgctgctgccggcagttccgtccctgggtgatggtggtggttgcgcactgccggttagcggtgcagcacagtgggctccggttctggacttcgcaccgccgggtgcatccgcacacggtcccctgggtggtccggcgccgccgtcggcaccgccgccgccgccgccgccgccgccgcactccttcatcaaacagggaccgagctggggtggcgcggaactgcackaakaacagtacctgagcgcgttcaccgttcactcctccggtcaggttcactggcacggccggggcctgtcgctacgggcccctcggcccccctccgcccagccaggcgtcatccggccaggccaggatgtctcctagcgcgccctgcctgcccagccgcctcgagagccagcccgctacccgcaatcggggctacagcacggtcaccttcgacggggcgtccggctacggtcacacgccctcgcaccatgcggcgcagttctcsmaryyactcgttaggcgtgaggatcccatgggccagcagggtccgctgggtgagcagcagtgctcggcgccgcccccggcctgtggccgccacacccccgccgacagctgcgccggcagccaggctttgctgctgagggcgccctgtagcagcgacggtttataccaagtgacgtcccagcttgagtgcatggcctggagtcagatgagcctcggggccgccttamcgggccacakyacarggtacgagagcgatgatcacacaacgcccggcctctgcggagcccaatacagaatacacacgcacggtgccttcaggggcgttcagggtgtgcgccgtgtgcctggagtagccccgactcttgtacggtcggcatctgaggccagtgaggaacgccccctcatgtgtgcttacccaggctgcaataggaggtatctgaagctgccccgcttacagatgcacggtaggaagcacgctggtgagagaccataccagtgtgacttcaaggactgtggacggaggtttttctgctcagaccggctcaaaagacaccaggggaggcatacagatgtgaagccattccagcgtaagacctgtcagcgagggttctcccggcccaaccacctgaagacccacgccaggactcatgcaggtgaaaagccccccagctgtcggtggtcagattgtcagagaaagcctgcccggtcaagtgagttggtccgccatcgcgacatgcatcagaggggcatgaccgaactccagctggcgctttgaa

SEQ ID NO: 378 - DNA, 1291 bp, Homo sapiens
atgggctccgacgttcgtgacctaaacgcactgctgccggcagttccgtccccgggtggtggtggtggttgcgcactgccggttagcggtgcaacacagtgggctccggttctggacttcgtaccgccgggtgcgcctgtatgcggttccctgggtggcccggcaccgccgccagcgccgccgccgctgccgccgccgccgtcgcactccttcaccaaacaggaaccgagttggggtggtacagagccgcacgcaggacagggccggagcgcactcgtcgctcactcctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcatccaagcatgaggaccccatgggccagcagggctcgccgggtgagcagcagtactcggcgccgcccccggtctgcggctgccgcacccccaccggcagctgcaccggcagccaggctttgctgctgagggcgccctacagcggtggcgatctacaccaaacgacatcccagcttggacacatggcctggaatcagacgaacttaggagccaccttaaagggccacggcacagggtacgagagcgatgaccacacaacgcccatcctctgcggaacccagtacaggatacgcgcgcgcggcgtcctccggggtactcaggatgtgcggtgtgtgcctggggtggccccgactcttgtgcggtcggcatctgagaccagtgagaagcgccccctcatgtgtgcctacccaggctgcaataagagacactttaagccgtcccgcttgcgggtgcgcggcagggagcgcactggtgagaaaccataccagcgcgacttcaaggaccgtggacgagggcttctccgtccagaccagctcaaaaggcaccagagggggcatacaggtgtgaaacctctccagtgtgaagcttgacggcggaggcccccccgacccggccacctgaaggtccacaccaggacccatacaggtggagagcccttcagttgtcggtggccaagttgtcaggagaagtctgcccggccagatgaatcagcccgccgtcataacatgcatcagagaaacatgaccaaactccagctggcgctttgaa

SEQ ID NO: 379 - DNA, 1281 bp, Homo sapiens
atgggctccgacgttcgtgacctgagtgcattgctaccgacggccccgtccctgggtggtggcggtgactgcacactgccggttagcggtacagcacagtgggctccggtcccggcctccgcaccgccgggcgcatccgcatacgattccctgggtggcccggcaccgccgccggcgccgccgccgccgccgccgccgccgccgcactcctgcggcgaacaggggccgagctggggtggtgcagaaccgcgcgaggggcaatgcctgagtgcgcccgccgtccgcttctccggccggttcaccggcacagtcggagcctgtcgctatgggcccctcggtcctcctccgcccagccaggcgccatccggccagaccaggatgttgcccagcgcgccctatctgtcgagttgcctcaggagccggtccgctatccgtagtcagggtcgcagcacggcaccttcagcggggcgcccagctatggcacccaccctcgcaccaccggcgcagtcccactactcccaacatggggtcctacatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtagcacctgagaccagtgagaacgccccttggtgtgtgttaccggggctgcagtaaggggtattttaagccgtcccacttacgggtgcacagcaggaagcgcattggtgagacgccacgccagtgcgactccaagggccgtggacgagggcctctccgttcgggaccagcccaagggacaccaaaggagacatacaggtacgcaaccactccagtgtaaggcttgtcagcgaaggttcccccggtccgaccacctgagggcccacgccagggcccacacgggtgggaagcccctcagctgccggtggccaagctgccagagagggttcgcccagtcagacgaattagtccgtcatcacaacatgtatcagcgaaacatgactaaactccagctggcgctttgaa

SEQ ID NO: 380 - DNA, 3020 bp, Homo sapiens
gttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagcctcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtctggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaagaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggactgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaaggaaaaaaaaaaaaaaaa

SEQ ID NO: 381 - DNA, 1291 bp, Homo sapiens
atgggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttgag

SEQ ID NO: 382 - DNA, 1491 bp, Homo sapiens
atggcggcccccggcgcccggcggtcgctgctcctgctgctgctggcaggccttgcacatggcgcctcagcactctttgaggatctaatgggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgcttcttaacaacatgttgatccccattgctgtgggcggtgccctggcagggctggtcctcatcgtcctcattgcctacctcattggcaggaagaggagtcacgccggctatcagaccatctagtga

SEQ ID NO: 383 - DNA, 1251 bp, Homo sapiens
atggcgccccgcagcgcccggcgacccctgctgctgctactgcctgttgctgctgctcggcctcatgcattgtcgtcagcagccatgtttatggtgaaaaatggcaacgggaccgcgtgcataatggccaacttctctgctgccttctcagtgaactacgacaccaagagtggccccaagaacatgacctttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcacgctgatccccatcgctgtgggtggtgccctggcggggctggtcctcatcgtcctcatcgcctacctcgtcggcaggaagaggagtcacgcaggctaccagactatctag

SEQ ID NO: 384 - DNA, 228 bp, Homo sapiens
atgcagatcttcgtgaagactctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtggg

SEQ ID NO: 385 - DNA, 1515 bp, Homo sapiens
atgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctagaagatggccgcactctttctgactacaacatccagaaggagtcgaccctgcacctggtccttcgcctgagaggtgccatgggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctatctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 386 - DNA, 648 bp, Homo sapiens
atgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcagtgatga

SEQ ID NO: 387 - DNA, 1089 bp, Homo sapiens
atgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 388 - DNA, 1035 bp, Homo sapiens
atgacggccgcgtccgataacttccagctgtcccagggtgggcagggattcgccattccgatcgggcaggcgatggcgatcgcgggccagatcaagcttcccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattccactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcagtga

SEQ ID NO: 389 - DNA, 1263 bp, Homo sapiens
atgacggccgcgtccgataacttccagctgtcccagggtgggcagggattcgccattccgatcgggcaggcgatggcgatcgcgggccagatcaagcttcccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcccgctggtgccgcgcggcagcccgatgggctccgacgttcgggacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcagtga

SEQ ID NO: 390 - DNA, 1707 bp, Homo sapiens
atgacggccgcgtccgataacttccagctgtcccagggtgggcagggattcgccattccgatcgggcaggcgatggcgatcgcgggccagatcaagcttcccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcccgctggtgccgcgcggcagcccgatgggctccgacgttcgggacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 391 - protein, 344 aa, Homo sapiens
MTAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPAEFHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQ

SEQ ID NO: 392 - protein, 568 aa, Homo sapiens
MTAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 393 - protein, 420 aa, Homo sapiens
MTAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQ

SEQ ID NO: 394 - protein, 362 aa, Homo sapiens
MHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 395 - protein, 214 aa, Homo sapiens
MHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQ

SEQ ID NO: 396 - DNA, 30 bp, Artificial Sequence
  PCR primer
gacgaaagcatatgcactccttcatcaaac

SEQ ID NO: 397 - DNA, 31 bp, Artificial Sequence
  PCR primer
cgcgtgaattcatcactgaatgcctctgaag

SEQ ID NO: 398 - DNA, 31 bp, Artificial Sequence
  PCR primer
cgataagcatatgacggccgcgtccgataac

SEQ ID NO: 399 - DNA, 31 bp, Artificial Sequence; identical to SEQ ID NO: 397
  PCR primer

SEQ ID NO: 400 - DNA, 31 bp, Artificial Sequence; identical to SEQ ID NO: 398
  PCR primer

SEQ ID NO: 401 - DNA, 28 bp, Artificial Sequence
  PCR primer
gtctgcagcggccgctcaaagcgccagc

SEQ ID NO: 402 - DNA, 30 bp, Artificial Sequence; identical to SEQ ID NO: 396
  PCR primer

SEQ ID NO: 403 - DNA, 28 bp, Artificial Sequence
  PCR primer
gtctgcagcggccgctcaaagcgccagc

SEQ ID NO: 404 - protein, 449 aa, Homo sapiens; identical to SEQ ID NO: 319

SEQ ID NO: 405 - protein, 428 aa, Homo sapiens
MGSDVRDLNALLPAVPSPGGGGGCALPVSGATQWAPVLDFVPPGAPVCGSLGGPAPPPAPPPLPPPPSHSFTKQEPSWGGTEPHAGQGRSALVAHSSGQFTGTAGACRYGPFGPPPPSQASSGQARMFPNAPYLPSCLESQPAIRNQGYSTVTFDGTPSYGHTPSHHAAQFPNHSSKHEDPMGQQGSPGEQQYSAPPPVCGCRTPTGSCTGSQALLLRAPYSGGDLHQTTSQLGHMAWNQTNLGATLKGHGTGYESDDHTTPILCGTQYRIRARGVLRGTQDVRCVPGVAPTLVRSASETSEKRPLMCAYPGCNKRHFKPSRLRVRGRERTGEKPYQRDFKDRGRGLLRPDQLKRHQRGHTGVKPLQCEARRRPPRPGHLKVHTRTHTGGEPFSCRWPSCQEKSARPDESARRHNMHQRNMTKLQLAL

SEQ ID NO: 406 - protein, 414 aa, Homo sapiens
  VARIANT 85, 86, 172, 173, 242, 245, 246, 247: Xaa = Any Amino Acid
MGSDVRDLSALLPAVPSLGDGGGCALPVSGAAQWAPVLDFAPPGASAHGPLGGPAPPSAPPPPPPPPPHSFIKQGPSWGGAELHXXQYLSAFTVHSSGQVHWHGRGLSLRAPRPPSAQPGVIRPGQDVSRALPAQPPREPARYPQSGLQHGHLRRGVRLRSHALAPCGAVLXXTRAGSHGPAGSAGAAVLGAAPGLWPPHPRRQLRRQPGFAAEGALQRRFIPSDVPAVHGLESDEPRGRLXGPXXXVRERSHNARPLRSPIQNTHARCLQGRSGCAPCAWSSPDSCTVGIGQGTPPHVCLPRLQEVSEAAPLTDAREARWETIPVLQGLWTEVFLLRPAQKTPGEAYRCEAIPADLSARVLPAQPPEDPRQDSCRKAPQLSVVRLSEKACPVKVGPPSRHASEGHDRTPAGAL

SEQ ID NO: 407 - protein, 417 aa, Homo sapiens
MGSDVRDLSALLPTAPSLGGGGDCTLPVSGTAQWAPVPASAPPGASAYDSLGGPAPPPAPPPPPPPPPHSCGEQGPSWGGAEPREGQCLSAPAVRFSGRFTGTVGACRYGPLGPPPPSQAPSGQTRMLPSAPYLSSCLRSRSAIRSQGRSTAPSAGRPAMAPTLAPPAQSHYSQHGVLHGPAGLAGAAVLGAAPGLWLPHPHRQLHRQPGFAAEDALQQQFIPNDIPAMHDLESDELRSHLKGPQHRVRERPHNAHPLRSPIQNTHARCLQRHSGCATCAWSSPDSCTVAPETSENAPWCVLPGLQGVFAVPLTGAQQEAHWDATPVRLQGPWTRASPFGTSPRDTKGDIQVRNHSSVRLVSEGSPGPTTGPTPGPTRVGSPSAAGGQAAREGSPSQTNSVITTCISETLNSSWRFE

SEQ ID NO: 408 - protein, 429 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 409 - protein, 495 aa, Homo sapiens
MAAPGARRSLLLLLLAGLAHGASALFEDLMGSDVRDLNALLPAVPSLGGGGGCALPVSGAAQWAPVLDFAPPGASAYGSLGGPAPPPAPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLALLNNMLIPIAVGGALAGLVLIVLIAYLIGRKRSHAGYQTI

SEQ ID NO: 410 - protein, 504 aa, Homo sapiens
MQIFVKTLTGKTITLEVEPSDTIENVKAKIQDKEGIPPDQQRLIFAGKQLEDGRTLSDYNIQKESTLHLVLRLRGAMGSDVRDLNALLPAVPSLGGGGGCALPVSGAAQWAPVLDFAPPGASAYGSLGGPAPPPAPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 411 - protein, 10 aa, Homo sapiens
VLDFAPPGAS

SEQ ID NO: 412 - protein, 15 aa, Homo sapiens
QWAPVLDFAPPGASA

SEQ ID NO: 413 - protein, 15 aa, Homo sapiens
VLDFAPPGASAYGSL